(19) United States
(12) Patent Application Publication
(10) Pub. No.: US 2007/0259965 A1
(43) Pub. Date: Nov. 8, 2007

(54) Acyclic 1,3-Diamine And Uses Therefor

(75) Inventors: Linda N. Casillas, Collegeville, PA (US); Jae Uk Jeong, Collegeville, PA (US); Robert W. Marquis, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SmithKline Beecham Corporation

(21) Appl. No.: 11/574,754

(22) PCT Filed: Sep. 7, 2005
(86) PCT No.: PCT/US05/31873

§ 371 Date: Mar. 6, 2007

Related U.S. Application Data
(60) Provisional application No. 60/607,678, filed on Sep. 7, 2004.

(51) Int. Cl. C07D 333/70

(52) U.S. Cl. 514/657; 514/646; 546/112; 549/49; 564/305

(57) ABSTRACT

This invention relates to novel compounds useful in the treatment of diseases associated with TRPV4 channel receptor. More specifically, this invention relates to certain substituted amino-azepines, according to Formula I 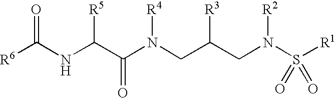 or pharmaceutically acceptable salts, hydrates, or solvates thereof, wherein:
R¹ is aryl optionally substituted with CN, NO₂, halogen or H;
R² is H, C₁-C₆ alkyl, C₃-C₇ cycloalkyl, or C₃-C₇ heterocycloalkyl;
R³ is H, OH, O—C₁-C₆ alkyl, SH, S—C₁-C₆ alkyl, or F;
R⁴ is H, C₁-C₆ alkyl
R⁵ is iso-butyl, cyclohexylmethyl, or cyclopentylmethyl; and
R⁶ is aryl, heteroaryl.

### FIELD OF THE INVENTION

This invention relates to novel compounds useful in the treatment of diseases associated with TRPV4 channel receptor. More specifically, this invention relates to certain acyclic diamines, which are agonists of TRPV4 channel receptors.

### BACKGROUND OF THE INVENTION

Cartilage is an avascular tissue populated by specialized cells termed chondrocytes, which respond to diverse mechanical and biochemical stimuli. Cartilage is present in the linings of joints, interstitial connective tissues, and basement membranes, and is composed of an extracellular matrix comprised of several matrix components including type II collagen, proteoglycans, fibronectin and laminin.

In normal cartilage, extracellular matrix synthesis is offset by extracellular matrix degradation, resulting in normal matrix turnover. Depending on the signal(s) received, the ensuing response may be either anabolic (leading to matrix production and/or repair) or catabolic (leading to matrix degradation, cellular apoptosis, loss of function, and pain).

TRPV4 channel receptor is one of six known members of the vanilloid family of transient receptor potential channels and shares 51% identity at the nucleotide level with TRPV1, the capsaicin receptor. Examples of polypeptides and polynucleotides encoding forms of human vanilloid receptors, including TRPV4 channel receptor from human can be found in EP 1170365 as well as WO 00/32766. Like the other family members TRPV4 channel receptor is a Ca2+ permeable, non-selective, ligand-gated cation channel, which responds to diverse stimuli such as reduced osmolality, elevated temperature, and small molecule ligands. See, for instance, Voets, et al., J. Biol. Chem. (2002) 277 33704-47051; Watanabe, et al., J. Biol. Chem. (2002) 277:47044-47051; Watanabe, et al., J. Biol. Chem. (2002) 277:13569-47051; Xu, et al., J. Biol. Chem. (2003) 278:11520-11527. From a screen of body tissues, the human TRPV4 channel receptor is most prominently expressed in cartilage. A screen of primary and clonal cell cultures shows significant expression only in chondrocytes.

In response to injurious compression and/or exposure to inflammatory mediators (e.g. inflammatory cytokines) chondrocytes decrease matrix production and increase production of multiple matrix degrading enzymes. Examples of matrix degrading enzymes include aggrecanases (ADAMTSs) and matrix metalloproteases (MMPs). The activities of these enzymes results in the degradation of the cartilage matrix. Aggrecanases (ADAMTSs), in conjunction with MMPs, degrade aggrecan, an aggregating proteoglycan present in articular cartilage. In osteoarthritic (OA) articular cartilage, a loss of proteoglycan staining is observed in the superficial zone in early OA and adjacent to areas of cartilage erosion in moderate to severe OA. The reduction in proteoglycan content is associated with an increase in degradation of type II collagen by specialized MMPs, termed collagenases (e.g. MMP-13). Collagenases are believed to make the initial cleavage within the triple-helix of intact collagen. It is hypothesized that the initial cleavage of collagen by collagenases facilitates the further degradation of the collagen fibrils by other proteases; accordingly, preventing or reducing the increased production of matrix degrading enzymes and/or attenuating the inhibition of matrix production may also promote functional recovery. Modulation of TRPV4 channel receptor has been shown to play a role in attenuating cartilage breakdown and matrix degrading enzymes.

Excessive degradation of extracellular matrix is implicated in the pathogenesis of many diseases, including chronic, neuropathic, and postoperative pain; rheumatoid arthritis; osteoarthritis; neuralgia; neuropathies; algesia; nerve injury; ischaemia; neurodegeneration; cartilage degeneration; stroke; incontinence; inflammatory disorders; irritable bowel syndrome; obesity; periodontal disease; aberrant angiogenesis; tumor invasion and metastasis; corneal ulceration; and complications of diabetes.

Thus, there is a need to discover new compounds useful in modulating TRPV4 channel receptors.

### SUMMARY OF THE INVENTION

This invention relates to a class of acyclic 1,3-diamines that can be used to treat diseases associated with TRPV4 channel receptors. This invention also relates to a pharmaceutical composition comprising a class of acyclic 1,3-diamines and a pharmaceutically acceptable carrier. In yet another aspect, this invention also relates to a method of treating diseases associated with TRPV4 channel receptor in mammals, particularly in humans.

Specifically, the invention is directed to compounds according to Formula I: 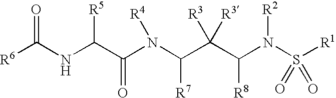
or a pharmaceutically acceptable salt thereof, or a solvate thereof, or a combination thereof, wherein:
R¹ is phenyl, thienyl, furanyl, benzoxadiazolyl, imidazo[2,1-b][1,3]thiazolyl, C₃-C₇ cycloalkyl-C₁-C₄ alkylenyl, C₃-C₇ cycloalkyloxy-C₁-C₄ alkylenyl, or N-ethenyl-tetrahydroindolyl, wherein R¹ is optionally substituted with one or more substituents selected from the group consisting of C₁-C₆ alkyl, C₁-C₆ alkylsulfonyl, [(methylamino)carbonyl]amino, cyano, nitro, trifluoromethyl, trifluoromethoxy, carboC₁-C₆alkyloxy, and halo;
R² is H, C₁-C₆ alkyl, halo C₁-C₆ alkyl, diC₁-C₆ alkylamino-C₁-C₆ alkylenyl, C₁-C₆ alkyloxy-C₁-C₆ alkylenyl, C₁-C₆ alkyloxy, pyridinyl-C₁-C₆ alkylenyl, C₃-C₇ cycloalkyl, or tetrahydropyranyl;
R³ is H, hydroxy, —O—C₁-C₆ alkyl, —SH, —S—C₁-C₆ alkyl, amino, C₁-C₄ alkylamino, propenyloxy, or halo;
R^{3′} is H or C₁-C₆ alkyl, or R^{3′} together with R³ forms an oxo group;
R⁴ is H or C₁-C₆ alkyl;
R⁵ is iso-butyl, 3,3-dimethylbutyl, thiazolylmethylenyl, hydroxyethylenyl, dichloroethyl, piperidinylmethylenyl, tetrahydropyranylmethylenyl, cyclopropylmethylenyl, cyclohexylmethylenyl, or cyclopentylmethylenyl;
R⁶ is phenyl, phenyl-C₁-C₄-alkylenyl, thienyl, benzo[b]thienyl, benzo[b]furanyl, thieno[2,3-b]pyridinyl, thieno[3,2-b]thienyl, furo[3,2-b]pyridinyl, benzodiazinyl, imidazo[1,2-b]pyridazinyl, indolyl, thienyl-C₁-C₄-alkylenyl, cyclopenta[b]thienyl, C₃-C₇ cycloalkyl, C₃-C₇ cycloalkyl-C₁-C₄ alkylenyl, C₃-C₇ cycloalkyloxy-C₁-C₄ alkylenyl, C₃-C₇ cycloalkylamino, C₁-C₆-alkylamino, C₁-C₆-dialkylamino, N-ethenyl-tetrahydroindolyl, tetrahydroisoquinolinyl, phenylmethyltetrahydroisoquinolinyl, phenylcarbonyltetrahydroisoquinolinyl, or 1,1-dimethylethyldihydroisoquinolincarboxylate-yl, bicyclo[2.2.1]hept-2-yl-C₁-C₄ alkylenyl, wherein R⁶ is optionally substituted with one or more substituents selected from the group consisting of halo, C₁-C₄-alkyl, phenyl, halophenyl, and amino;
R⁷ is H or C₁-C₆ alkyl; and
R⁸ is H, C₁-C₆ alkyl, COOH, acetylamino-C₁-C₄ alkylenyl, or hydroxymethyl.

### DETAILED DESCRIPTION OF THE INVENTION

Abbreviations and symbols utilized herein are in accordance with the common usage of such abbreviations and symbols by those skilled in the chemical arts. For example, “EDC” means N-ethyl-N′(dimethylaminopropyl)-carbodiimide, “HOOBt” refers to hydroxy-3,4-dihydroxy-4-oxo-1,2,3-benzotriazine, “DMF” means dimethyl formamide, “DMSO” means dimethyl sulfoxide, “TEA” means triethylamine, and “THF” means tetrahydrofuran.

### Terms and Definitions

As used herein, “acyclic 1,3-diamines” refer to compounds having two nitrogen atoms separating three optionally substituted carbon atoms. By way of example, the following fragments constitute acyclic 1,3-diamines:

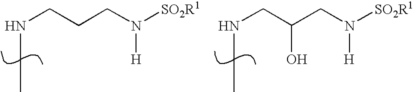

The term “C₁-C₆ alkyl” is used herein to refer to a straight or branched chain monovalent radical of 1 to 6 carbon atoms, including methyl, ethyl, n-propyl, isopropyl, n-butyl, isobutyl, t-butyl, pentyl, n-pentyl, isopentyl, neopentyl, and n-hexyl and isomers thereof.; (similarly, C₁-C₄ alkyl means a radical of 1 to 4 carbon atoms).

Similarly, the term “C₃-C₇ cycloalkyl” is used herein to a saturated monovalent cyclic ring of 3 to 7 carbon atoms, including cyclopropyl, cyclobutyl, cyclopentyl, cyclohexyl, and cycloheptyl.

The term “C₁-C₆ alkylenyl” refers to a straight or branched chain divalent radical of 1 to 6 carbon atoms, including but not limited to —CH₂—, —CH₂CH₂—, —CH₂CH₂CH₂—, —CH(CH₃)CH₂CH₂—, —CH(CH₃)CH₂—, —C(CH₃)₂CH₂— and —CH₂CH₂CH₂CH₂CH₂CH₂—. Similarly, “C₁-C₄ alkylenyl” refers to a divalent radical of 1 to 4 carbon atoms. As used herein, the term hydroxyalkyl (e.g., hydroxymethyl) is interchangeable with the term hydroxyalkylenyl; similarly, haloalkyl (e.g., dichloromethyl, trifluoromethyl, and 2,2,2,-trifluoroethyl) is interchangeable with haloalkylenyl.

Phenyl groups may be optionally substituted with groups, generally up to 3 groups, selected from halo including F, Cl, and Br; haloC₁-C₆ alkyloxy including trifluoromethoxy; haloC₁₋₆ alkyl including trifluoromethyl; C₁-C₆ alkyl, C₁-C₆ alkylsulfonyl; C₂-C₄alkenyloxy including ethenyloxy and propenyloxy; carboC₁-C₆alkyloxy including carbomethoxy; hydroxy; hydroxyC₁-C₆ alkylenyl including HO—CH₂—, and HO—CH₂CH₂—; C₁-C₆alkoxy including methoxy; nitro; cyano; amino; aminocarbonyl; C₁-C₆ alkylaminocarbonylamino, and C₁-C₆dialkylaminocarbonylamino.

The alkyl and cycloalkyl groups may be unsubstituted or substituted. Unless otherwise defined, suitable substituents for any C₁-C₆ alkyl, and C₃-C₇ cycloalkyl groups include substituents selected from the group consisting of hydroxy, halo, nitro, cyano, carboxy, amino, C₁-C₆ alkylamino, C₁-C₆ dialkylaminoC₁-C₆ alkyloxy, trifluoromethyl, acyloxy, C₃-C₇ cycloalkyl, phenyl, and C₃-C₇ heterocycloalkyl.

“Enantiomeric excess” or “ee” is the excess of one enantiomer over the other expressed as a percentage. As a result, since both enantiomers are present in equal amounts in a racemic mixture, the enantiomeric excess is zero (0% ee). Accordingly, if one enantiomer were enriched so as to constitute 95% of the product, then the ee would be 90% (the amount of the enriched enantiomer, 95%, minus the amount of the other enantiomer, 5%).

“Enantiomerically enriched” refers to products having enantiomeric excess (ee) of greater than zero. For example, enantiomerically enriched refers to products whose ee is greater than about 50%, greater than about 75%, and greater than about 90%.

“Enantiomerically pure” refers to products whose enantiomeric excess is 100%.

The term “Heterocycloalkyl” is used herein to refer to a stable monovalent saturated heterocyclic ring and consist of carbon atoms and from one to three heteroatoms selected from the group consisting of N, O and S, wherein N may optionally be oxidized or quaternized. Heterocycloalkyl may be optionally unsubstituted or substituted as defined herein. Compounds within the invention containing a heterocycloalkyl group may occur in two or more tautometric forms depending on the nature of the heterocycloalkyl group; all such tautomeric forms are included within the scope of the invention. Representative examples include pyrrolidinyl, tetrahydrofuranyl, dihydrofuranyl, tetrahydropyranyl, dihydropyranyl, tetrahydrothienyl, pyrazolyl, pyrazolinyl, oxazolinyl, thiazolinyl, piperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, and oxabicylo[2.2.1]heptyl.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, “agonist” to a TRPV4 channel receptor includes any compound capable of activating or enhancing the biological activities of a TRPV4 channel receptor.

As used herein, “activating” the TRPV4 channel receptor may include, but is not limited to, such outcomes as increasing the amount of Ca²⁺ influx into a cell comprising a TRPV4 channel receptor, reducing the amount of ADAMTSs produced and/or released by the cell, reducing the amount of MMPs produced and/or released by the cell, inhibiting the basal or growth factor-stimulated proliferation of the cell, reducing the amount of nitric oxide (NO) produced by a cell, and attenuating the inhibition of matrix synthesis.

As used herein, “inflammatory mediators” include any compound capable of triggering an inflammatory process. The term inflammation generally refers to the process of reaction of vascularized living tissue to injury. This process includes but is not limited to increased blood flow, increased vascular permeability, and leukocytic exudation. Because leukocytes recruited into inflammatory reactions can release potent enzymes and oxygen free radicals, the inflammatory response is capable of mediating considerable tissue damage. Examples of inflammatory mediators include, but are not limited to prostaglandins (e.g., PGE2), leukotrienes (e.g., LTB4), inflammatory cytokines, such as tumor necrosis factor alpha (TNFα), interleukin 1 (IL-1), and interleukin 6 (IL-6); nitric oxide (NO), metalloproteinases, and heat shock proteins.

As used herein “matrix protein” includes proteins released from cells to form the extracellular matrix of cartilage. The extracellular matrix of cartilage consists of proteoglycans, belonging to several distinct proteoglycan families. These include, but are not limited to, perlecan and the hyalectans, exemplified by aggrecan and versican, and the small leucine-rich family of proteoglycans, including decorin, biglycan and fibromodulin. The extracellular matrix also consists of hybrid collagen fibers comprised of three collagen isotypes, namely type II, type IX, and type XI collagens, along with accessory proteins such as cartilage oligeromeric matrix protein (COMP), link protein, and fibronectin. Cartilage also contains hyaluronin which forms a noncovalent association with the hyalectins. In addition, a specialized pericellular matrix surrounds the chondrocyte which consists of proteoglycans, type VI collagen and collagen receptor proteins, such as anchorin.

As used herein “matrix degrading enzymes” refers to enzymes capable of cleaving extracellular matrix proteins. Cartilage extracellular matrix turnover is regulated by matrix metalloproteases (MMPs) which are synthesized as latent proenzymes that require activation in order to degrade cartilage extracellular matrix proteins. Three classes of enzymes are believed to regulate the turnover of extracellular matrix proteins, namely collagenases (including, but not limited to, MMP-13), responsible for the degradation of native collagen fibers, stromelysins (including, but not limited to, MMP-3) which degrade proteoglycan and type IX collagen, and gelatinases (including, but not limited to, MMP-2 and MMP-9) which degrade denatured collagen. The matrix degrading enzyme group that appears most relevant in cartilage degradation in OA includes a subgroup of metalloproteinases called ADAMTS, because they possess disintegrin and metalloproteinase domains and a thrombospondin motif in their structure. ADAMTS4 (aggrecanase-1) has been reported to be elevated in OA joints and along with ADAMTS-5 (aggrecanase-2) have been shown to be expressed in human osteoarthritic cartilage. These enzymes appear to be responsible for aggrecan degradation without MMP participation. Thus, an inhibition of activity or a reduction in expression of these enzymes may have utility in OA therapy.

As used herein, “reduce” or “reducing” the production of matrix degrading enzymes refers to a decrease in the amount of matrix degrading enzyme(s) produced and/or released by a cell, which has exhibited an increase in matrix degrading enzyme production or release in response to a catabolic stimulus, which may include, but is not limited to, physical injury, mechanical and/or osmotic stress, or exposure to an inflammatory mediator.

As used herein “attenuate” or “attenuating” refers to a normalization (i.e., either an increase or decrease) of the amount of matrix degrading enzyme, inflammatory mediator, or matrix protein produced and/or released by a cell, following exposure to a catabolic stimulus. For example, following exposure to IL-1 chondrocyte production of matrix proteins, such as proteoglycans, are reduced, while production of matrix degrading enzymes (e.g. MMP-13, ADAMTS4) and reactive oxygen species (e.g. NO) are increased. Attenuation refers to the normalization of these diverse responses to levels observed in the absence of a catabolic stimulus.

The term “EC₅₀” is used herein to refer to the molar concentration of an agonist that produces 50% of the maximum possible response for that agonist.

Some of the compounds of this invention may be crystallized or recrystallized from solvents such as aqueous and organic solvents. In such cases solvates may be formed. This invention includes within its scope stoichiometric solvates including hydrates as well as compounds containing variable amounts of water that may be produced by processes such as lyophilisation.

Certain of the above-mentioned compounds of formula (I) may exist in the form of optical isomers including diastereoisomers, and mixtures of isomers in all ratios including racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

The composition may be formulated for administration by any route, such as oral, topical or parenteral. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration, and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents such as syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases such as cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilized before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compounds according to Formula I may contain one or more asymmetric center and may, therefore, exist as individual enantiomers, diasteriomers, or other stereoisomeric forms, or as mixtures thereof. For example, when R³, is not the same as R^{3′}, the carbon to which R³ and R^{3′} are attached is asymmetric. The same logic holds for when R⁵ is other than H. In addition, asymmetric carbon atoms may also be present in a substituent such as an alkyl group. Where the stereochemistry of chiral carbons present in Formula I, or in any chemical structure illustrated herein, is not specified, the chemical structure is intended to encompass compounds containing any stereoisomer and all mixtures thereof of each chiral center present in the compound. Thus, compounds according to Formula I containing one or more chiral center may be used as racemic mixtures, enantiomerically enriched mixtures, or as enantiomerically pure individual stereoisomers.

Individual stereoisomers of a compound according to Formula I which contain one or more asymmetric center may be resolved by methods known to those skilled in the art. For example, such resolution may be carried out by formation of diastereoisomeric salts or complexes which may be separated, for example, by crystallization; by formation of diastereoisomeric derivatives which may be separated, for example, by crystallization, gas-liquid or liquid chromatography; by selective reaction of one enantiomer with an enantiomer-specific reagent, for example by enzamatic oxidation or reduction, followed by separation of the modified and unmodified enantiomers; or gas-liquid or liquid chromatography in a chiral environment, for example, on a chiral support such as silica with a bound chiral ligand or in the presence of a chiral solvent. The skilled artisan will appreciate that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer to the other by asymmetric transformation.

The compounds according to Formula I may also contain double bonds or other centers of geometric asymmetry. Formula I includes both trans (E) and cis (Z) geometric isomers. Likewise, all tautomeric forms are also included in Formula I whether such tautomers exist in equilibrium or predominately in one form.

The skilled artisan will appreciate that pharmaceutically-acceptable salts of the compounds according to Formula I can be prepared. Indeed, in certain embodiments of the invention, pharmaceutically-acceptable salts of the compounds according to Formula I may be preferred over the respective free base or free acid because such salts impart greater stability or solubility to the molecule thereby facilitating formulation into a dosage form. Accordingly, the invention is further directed to pharmaceutically-acceptable salts of the compounds according to Formula I.

As used herein, the term “pharmaceutically-acceptable salts” refers to salts that retain the desired biological activity of the subject compound and exhibit minimal undesired toxicological effects. The term “pharmaceutically-acceptable salts” includes both pharmaceutically-acceptable acid addition salts and pharmaceutically-acceptable base addition salts. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group and are therefore capable of forming pharmaceutically-acceptable base addition salts by treatment with a suitable base. Suitable bases include ammonia and hydroxides, carbonates and bicarbonates of a pharmaceutically-acceptable metal cation, such as alkali metal and alkaline earth metal cations. Suitable metal cations include sodium, potassium, lithium, calcium, magnesium, aluminum, and zinc. Suitable bases further include pharmaceutically-acceptable organic primary, secondary, and tertiary amines including aliphatic amines, aromatic amines, aliphatic diamines, and hydroxy alkylamines. Suitable pharmaceutically-acceptable organic bases include methylamine, ethylamine, diethylamine, ethylenediamine, ethanolamine, diethanolamine, and cyclohexylamine.

In certain embodiments, compounds according to Formula I may contain a basic functional group and are therefore capable of forming pharmaceutically-acceptable acid addition salts by treatment with a suitable acid. Suitable acids include pharmaceutically-acceptable inorganic acids, pharmaceutically-acceptable organic acids, and pharmaceutically-acceptable organic sulfonic acids. Suitable inorganic acids include, but are not limited to, hydrochloric acid, hydrobromic acid, nitric acid, sulfuric acid, sulfamic acid, and phosphoric acid. Suitable organic acids include, acetic acid, hydroxyacetic acid, propionic acid, butyric acid, isobutyric acid, maleic acid, hydroxymaleic acid, acrylic acid, fumaric acid, malic acid, tartaric acid, citric acid, salicylic acid, p-aminosalicyclic acid, glycollic acid, lactic acid, heptanoic acid, phthalic acid, oxalic acid, succinic acid, benzoic acid, o-acetoxybenzoic acid, chlorobenzoic acid, methylbenzoic acid, dinitrobenzoic acid, hydroxybenzoic acid, methoxybenzoic acid, phenylacetic acid, mandelic acid, formic acid, stearic acid, ascorbic acid, palmitic acid, oleic acid, pyruvic acid, pamoic acid, malonic acid, lauric acid, glutaric acid, and glutamic acid. Suitable organic sulfonic acids include, methanesulfonic acid, ethanesulfonic acid, 2-hydroxyethanesulfonic acid, benzenesulfonic acid, p-aminobenzenesulfonic (i.e. sulfanilic acid), prtoluenesulfonic acid, and napthalene-2-sulfonic acid.

As used herein, the term “compounds of the invention” means both the compounds according to Formula I and the pharmaceutically-acceptable salts thereof. The term “a compound of the invention” also appears herein and refers to both a compound according to Formula I and its pharmaceutically-acceptable salts.

When in the solid state, the compounds of the invention may exist as either amorphous material or in crystalline form, or as a mixture thereof. The skilled artisan will appreciate that pharmaceutically-acceptable solvates of the compounds of the invention may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” The invention includes all such solvates.

Synthetic Schemes:

The synthesis of the compounds of the general formula (I) may be accomplished as outlined below in Schemes 1-20.

Treatment of a carboxylic acid such as benzothiophene-2-carboxylic acid 1 with N-hydroxysuccinimide 2 under conditions common to the art such as EDC in an organic solvent such as dichloromethane can be utilized to provide the activated ester 1-[(1-benzothien-2-ylcarbonyl)oxy]-2,5-pyrrolidinedione 3. Subsequent treatment with leucine provides the peptide product 4. Standard peptide coupling conditions common to the art such as EDC and HOOBt in the presence of a base such as N-methyl-morpholine can be employed in the presence of an amine such as 1,1-dimethylethyl (3-aminopropyl)methylcarbamate to provide the amide product 5. Removal of the tert-butyl carbonyl group under conditions common to the art such as HCl or TFA followed by treatment of the free amine 6 with an electrophilic reagent such as 2-chloro-4-cyanobenzenesulfonyl chloride provides the final compound 7.

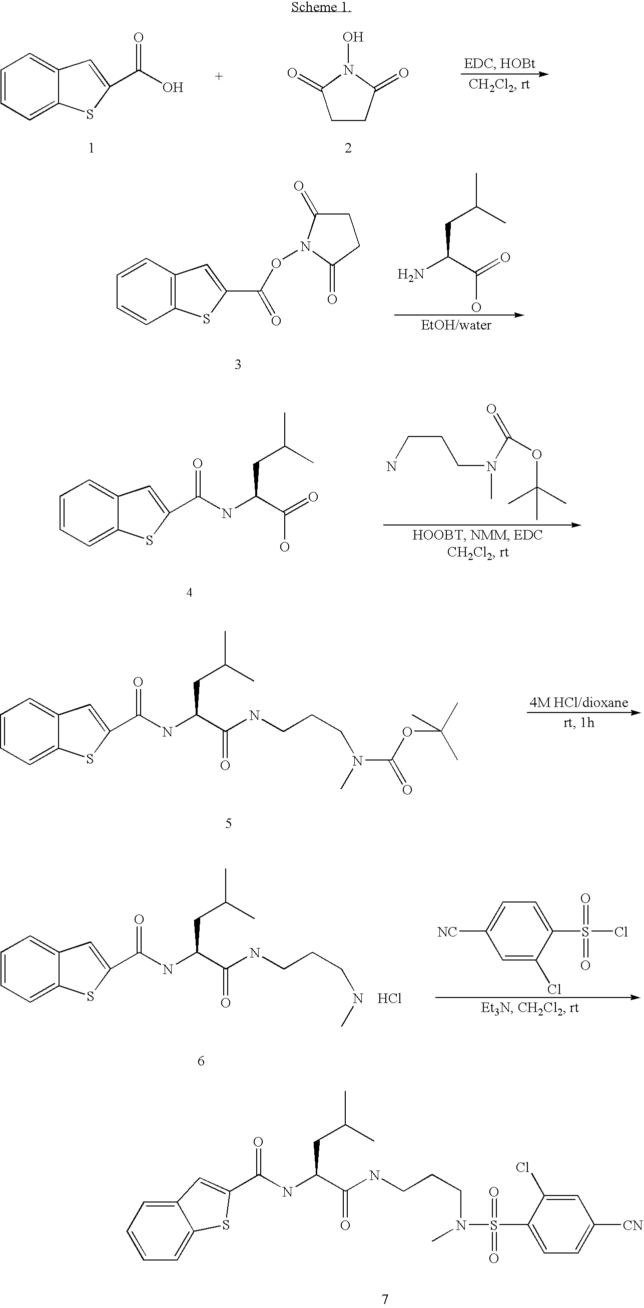

As outlined in Scheme 2, protection of cyclohexylalanine 8 can be accomplished under conditions common to the art such as benzoyl chloroformate and a base such as potassium carbonate in a biphasic solvent system such as THF and water to provide acid 9. Standard peptide coupling conditions common to the art such as EDC and HOOBt in the presence of a base such as N-methyl-morpholine can be employed in the presence of an amine such as 1,1-dimethylethyl (3-aminopropyl)methylcarbamate to provide the amide product 10. The CBZ group can be removed using conditions common to the art such as hydrogen and palladium on carbon under pressure to provide the primary amine 11. Standard peptide coupling conditions common to the art such as EDC and HOOBt in the presence of a base such as N-methyl-morpholine can be employed in the presence of an acid such as benzothiophene-2-carboxylic acid to provide the amide product 12. Removal of the tert-butyl carbonyl group under conditions common to the art such as HCl or TFA followed by treatment of the free amine 13 with an electrophilic reagent such as 2-bromo-4-fluorobenzenesulfonyl chloride provides the final compound 14.

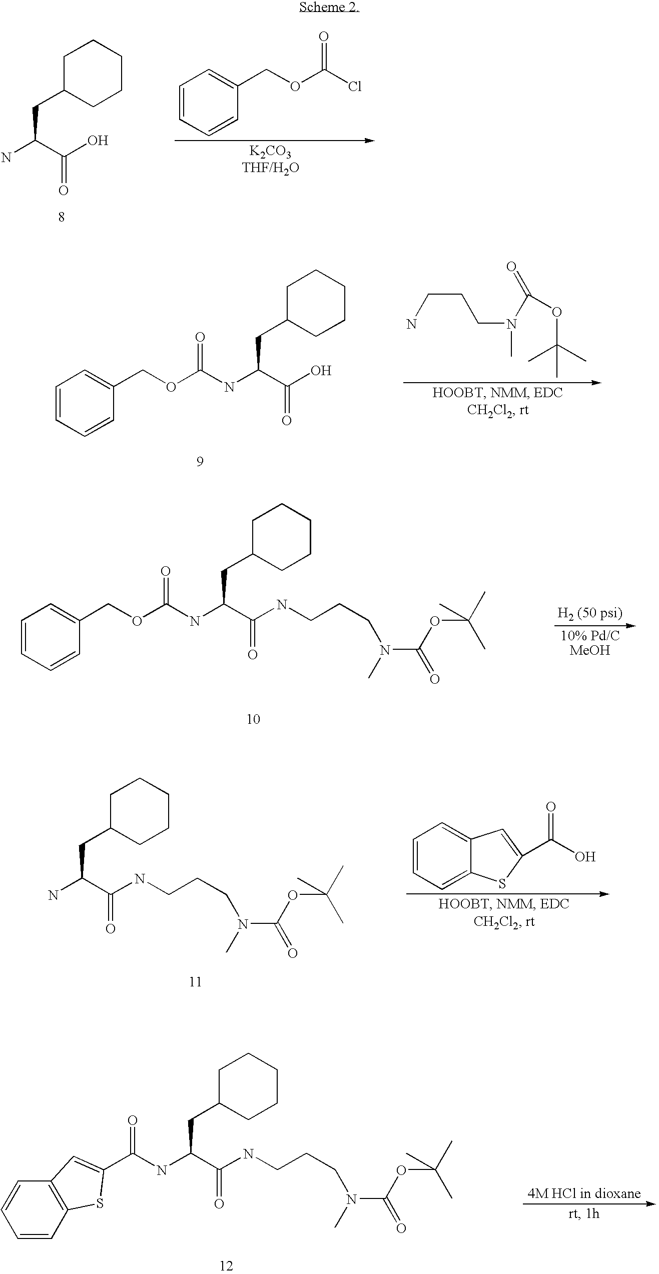

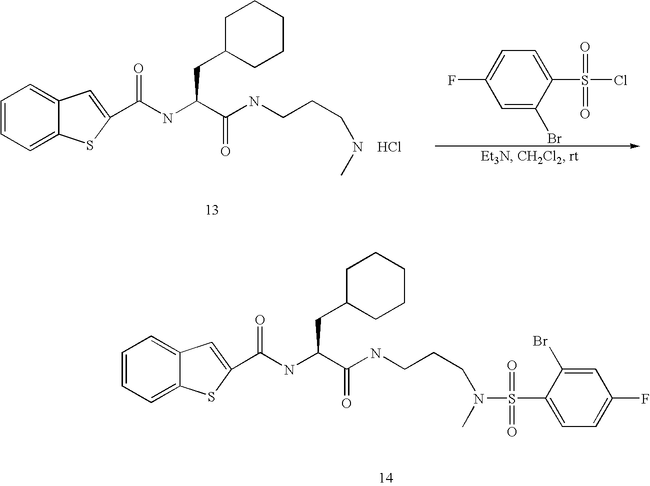

The sulfonamide-leucinamide portion can be constructed using an alternate sequence of steps as depicted in Scheme 3 such that CBZ-Leu can be coupled to 1,1-dimethylethyl (3-aminopropyl)methylcarbamate under conditions common to the art such as EDC and HOOBt in the presence of a base such as N-methyl-morpholine to give the peptide product 16. Removal of the tert-butyl carbonyl group under standard conditions such as HCl or TFA provides the free amine 17 which can be treated with an electrophilic reagent such as 2-chloro-4-fluorobenzene sulfonyl chloride to give the sulfonamide 18. Removal of the CBZ group can be accomplished by treatment with boron tribromide followed by an aqueous work up to provide N¹-{3-[[(2-chloro-4-fluorophenyl)sulfonyl](methyl)amino]propyl}-L-leucinamide 19 which can be incorporated into other target compounds.

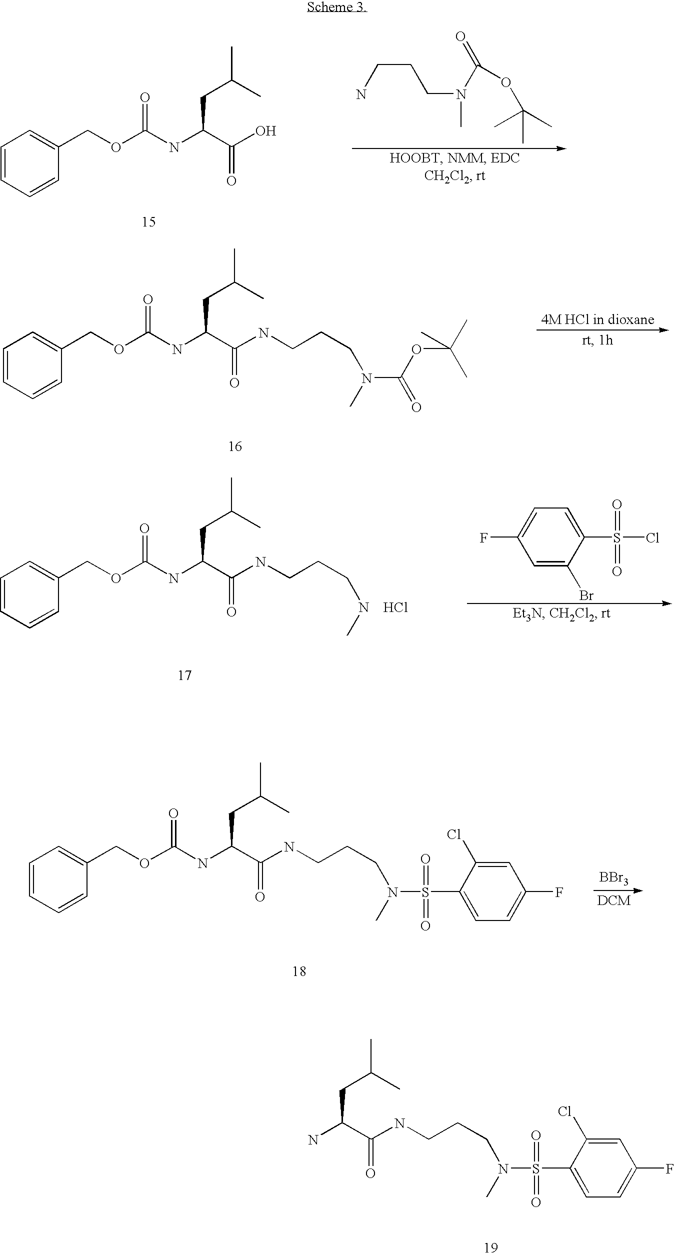

As shown in Scheme 4, some targets may be accessed by the following route. Treatment of 1,1-dimethylethyl (3-aminopropyl)carbamate 20 with an electrophilic reagent such as 4-fluoro-2-trifluoromethylbenzene sulfonyl chloride results in the formation of sulfonamide 21. Removal of the tert-butyl carbonyl group under standard conditions such as HCl or TFA provides the free amine 22 which can be coupled to Boc-phenylglycine under conditions common to the art such as EDC and HOOBT in the presence of a base such as N-methyl-morpholine to provide peptide 23. Again, removal of the tert-butyl carbonyl group under standard conditions such as HCl or TFA provides the free amine 24 which can be coupled to a carboxylic acid such as cyclopentyl propionic acid under conditions common to the art such as EDC and HOOBT in the presence of a base such as N-methyl-morpholine to provide the final compound 25.

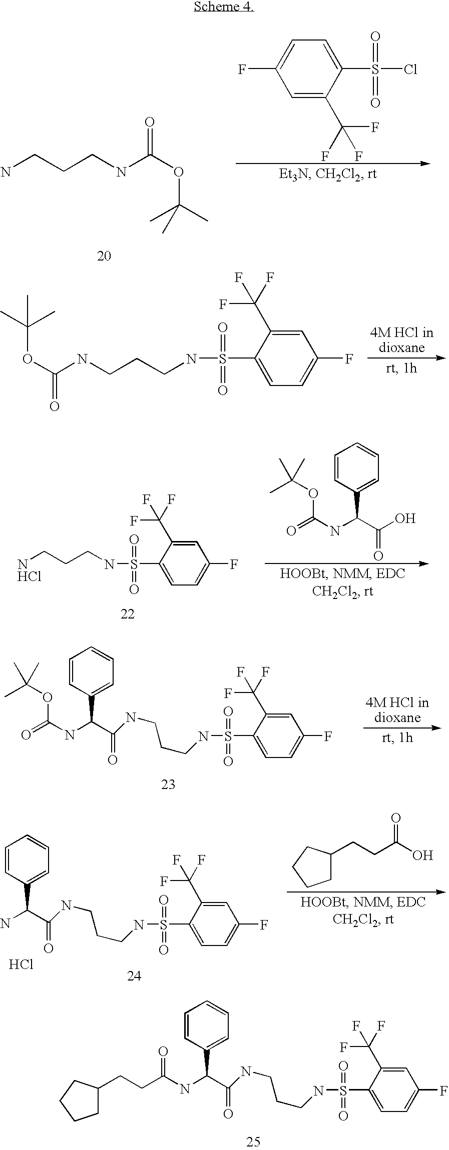

Elaboration of compound 26 can be accomplished as delineated in Scheme 5. Removal of the tert-butyl carbonyl group under standard conditions such as HCl or TFA provides the free amine 27. The product amine can be treated with an electrophilic reagent such as benzyl chloride under conditions common to the art in the presence of a base such as triethylamine to provide the benzylamine product 28.

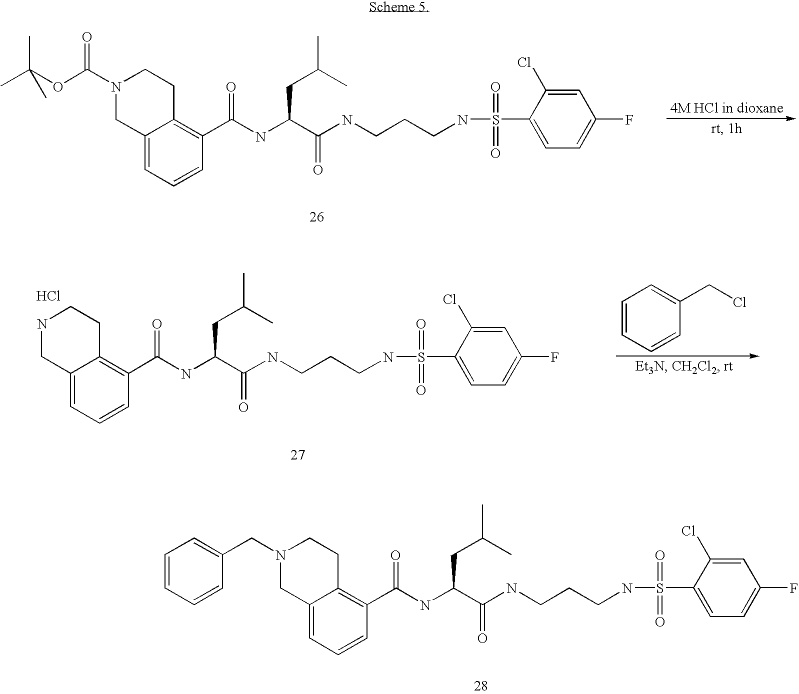

As shown in Scheme 6, some targets may be accessed by the following route. Treatment of N-methyl-1,3-propanediamine 29 with an electrophilic reagent such as 2-cyanobenzene sulfonyl chloride results in the formation of sulfonamide 31. Standard peptide coupling conditions common to the art such as EDC and HOOBt in the presence of a base such as N-methyl-morpholine can be employed in the presence of a carboxylic acid such as N-(1-benzothien-2-ylcarbonyl)-L-leucine to provide the amide product 32. Alternatively, various Boc-protected amino acids may be coupled to N-(3-aminopropyl)-2-cyano-N-methylbenzenesulfonamide 31 under conditions common to the art such as EDC and HOBt in the presence of a base such as N-methyl-morpholine to provide the generic Boc-protected product 33. Removal of the tert-butyl carbonyl group under conditions common to the art such as HCl or TFA provides the free amine and subsequent be coupling to a carboxylic acid under standard peptide coupling conditions such as EDC and HOBt in the presence of a base such as N-methyl-morpholine provides the representative peptide product 34.

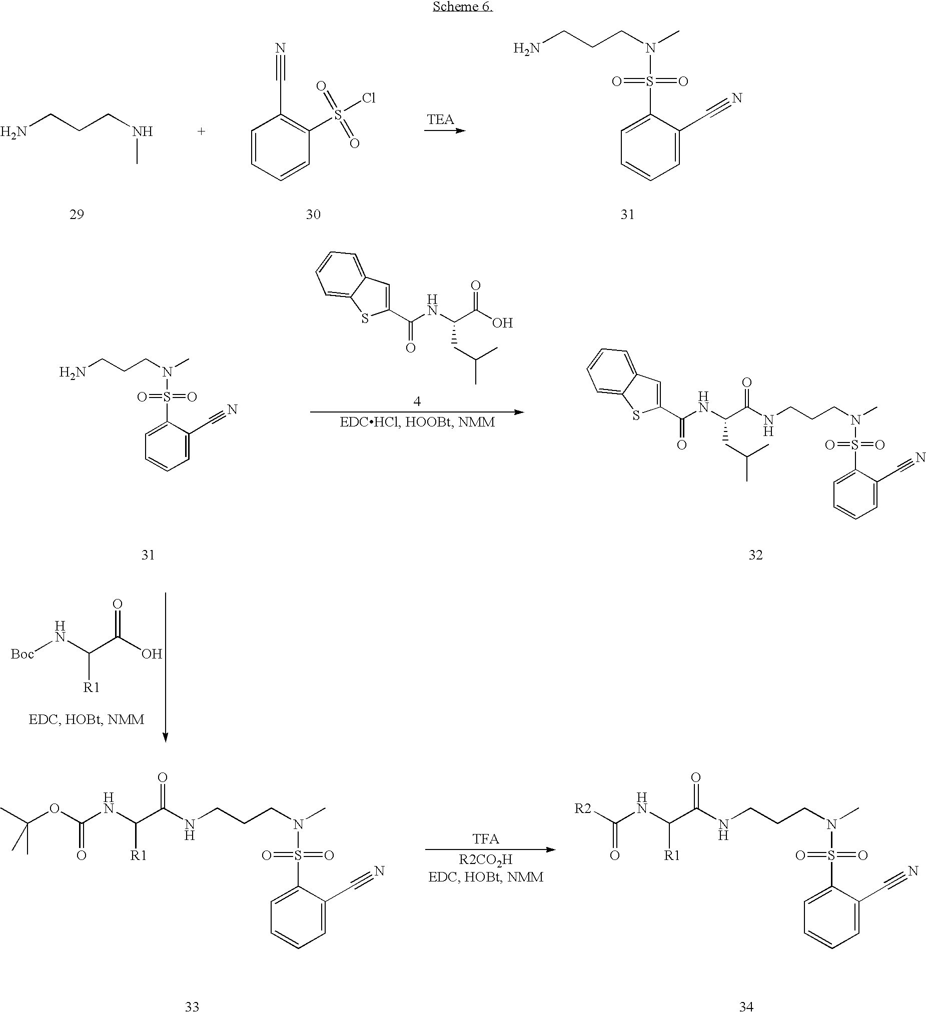

Scheme 7 depicts the route used to provide various N-substituted sulfonamide targets. Treatment of a diamine such as 35 with and activated ester such as 4-nitrophenyl N-{[(1,1-dimethylethyl)oxy]carbonyl}-L-leucinate under conditions common to the art provides the free secondary amine 37. Subsequent treatment with an electrophilic reagent such as 2-cyanobenzene sulfonamide provides sulfonamides of the general structure 38. Removal of the tert-butyl carbonyl group under conditions common to the art such as HCl or TFA provides the free amine and subsequent be coupling to a carboxylic acid such as benzothiophene-2-carboxylic acid under standard peptide coupling conditions such as EDC and HOOBt in the presence of a base such as N-methyl-morpholine provides the representative peptide product 40.

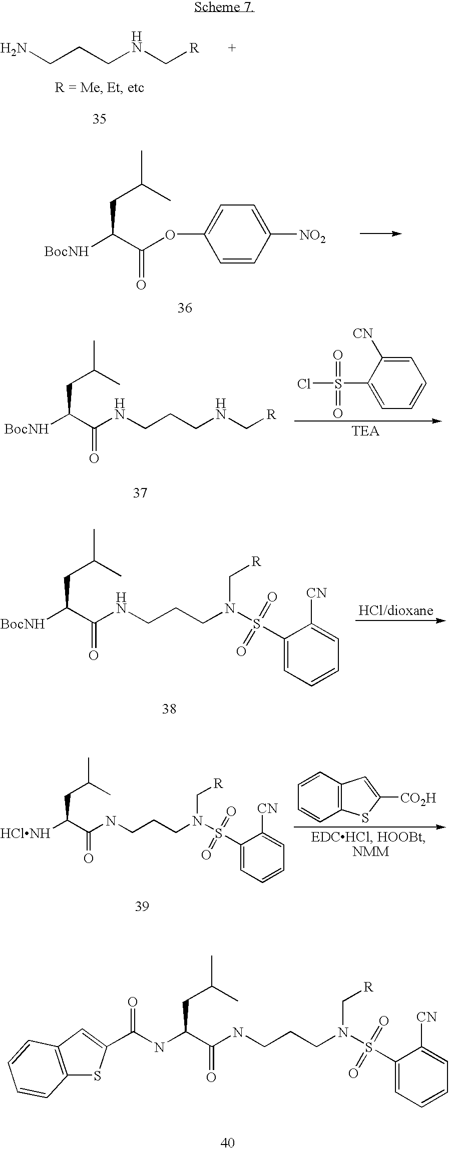

An alternative approach to providing N-substituted sulfonamide targets is adumbrated in Scheme 8. 3,3-Bis(ethyloxy)-1-propanamine 41 can be coupled to a carboxylic acid such as N-(1-benzothien-2-ylcarbonyl)-L-leucine under standard peptide coupling conditions such as EDC and HOOBt in the presence of a base such as N-methyl-morpholine to provide the peptide 42. Removal of the diethylacetal can be accomplished under conditions common to the art such as toluenesulfonic acid and wet acetone to provide the aldehyde 43. Treatment of this aldehyde with an amine such as cyclopropylamine under reductive amination conditions common to the art such as sodium cyanoborohydride in the presence of an acid such as HCl provides the amine product 44. Treatment of the amine with an electrophilic reagent such as 2-cyanobenzene sulfonyl chloride in the presence of an amine base such as triethylamine provides the target sulfonamide 45.

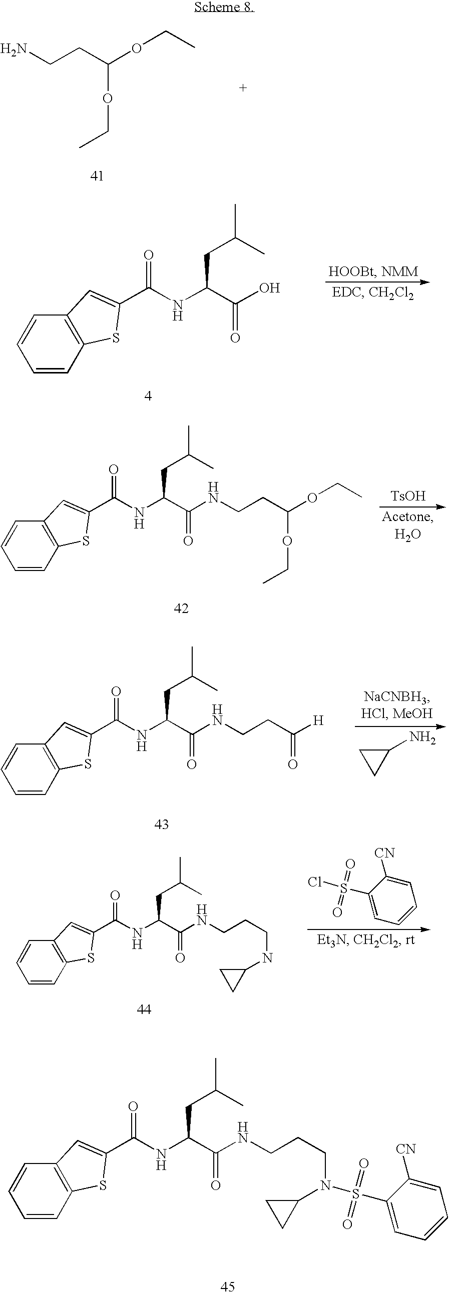

As outlined in Scheme 9, 1,1-dimethylethyl (3-aminopropyl)carbamate 20a can be coupled to a carboxylic acid such as N-(1-benzothien-2-ylcarbonyl)-L-leucine under standard peptide coupling conditions such as EDC and HOOBt in the presence of a base such as N-methyl-morpholine to provide the peptide 46a. Removal of the tert-butyl carbonyl group can be accomplished under standard conditions such as HCl or TFA to provide amine 6. Alternatively, if a nosyl-protected amine is used in the first step (20b), then removal of the nosylate group can be accomplished under conditions common to the art such as potassium carbonate and thiophenol to provide the amine 6. Treatment of the amine with an electrophilic reagent such as a sulfonyl chloride in the presence of an amine base such as triethylamine provides the representative sulfonamide 47.

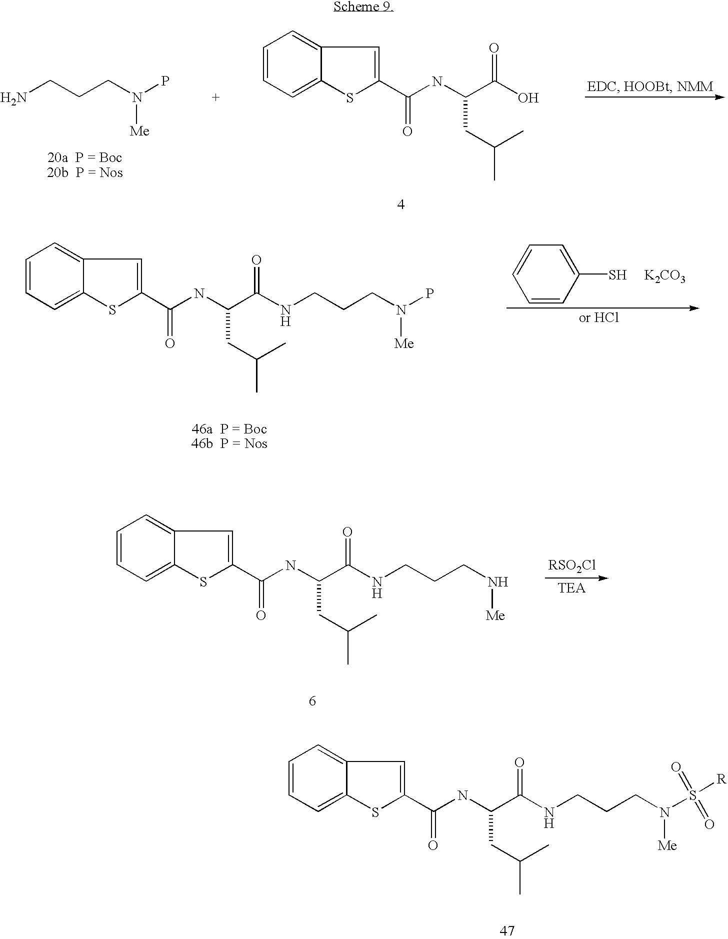

As outlined in Scheme 10, 1,1-dimethylethyl (3-aminopropyl)carbamate 20 can be coupled to a carboxylic acid such as N-(1-benzothien-2-ylcarbonyl)-L-leucine 4 under standard peptide coupling conditions such as EDC and HOOBt in the presence of a base such as N-methyl-morpholine to provide the peptide 48. Removal of the tert-butyl carbonyl group can be accomplished under standard conditions such as HCl or TFA to provide amine which may be treated with an electrophilic reagent such as 2-chloro-4-fluorobenzenesulfonyl chloride in the presence of an amine base such as triethylamine provides the sulfonamide target 49.

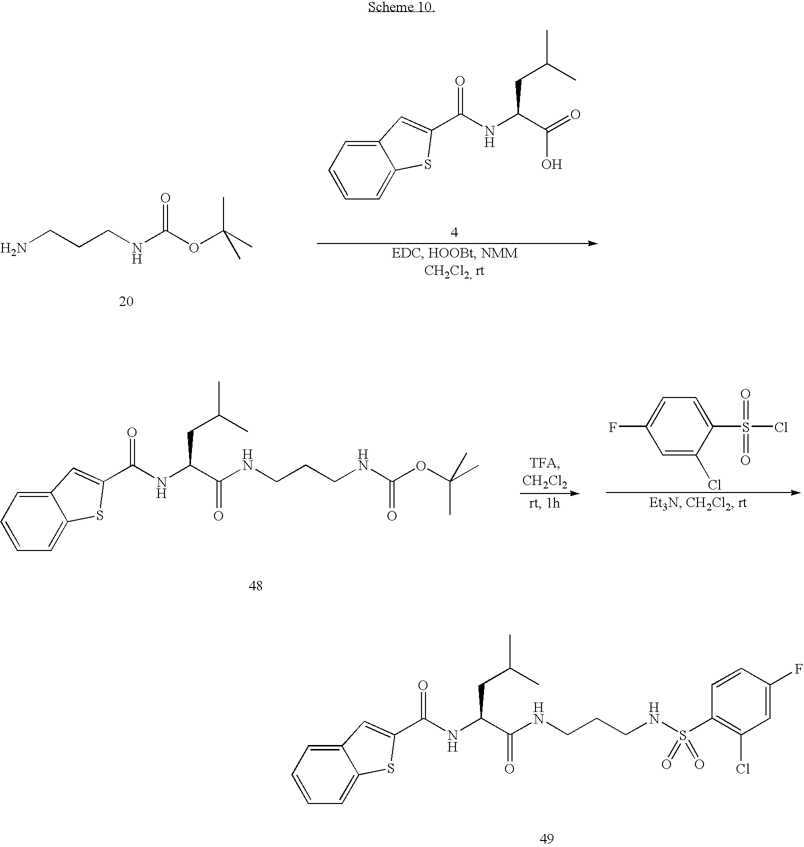

As shown in Scheme 11, some targets may be accessed by the following route. Treatment of a diamine represented by structure 50 with an electrophilic reagent such as 2-cyanobenzene sulfonyl chloride results in the formation of a representative sulfonamide 51. Standard peptide coupling conditions common to the art such as EDC and HOOBt in the presence of a base such as N-methyl-morpholine can be employed in the presence of a carboxylic acid such as N-[(1-methyl-1H-indol-2-yl)carbonyl]-L-leucine to provide the representative amide product 52.

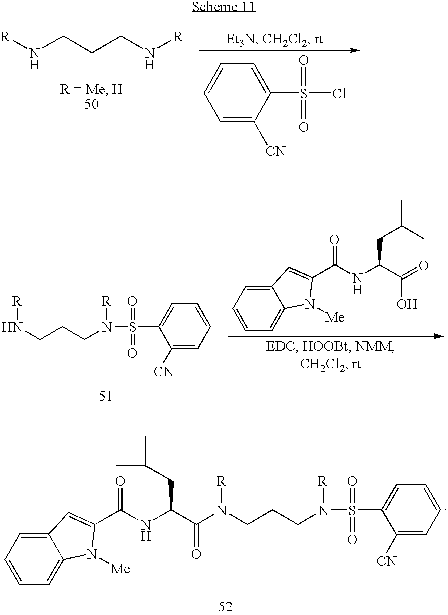

As outlined in Scheme 12, target compounds may also be prepared by first treating 2-nitrobenzene sulfonyl chloride (nosyl chloride) 53 with N-methylamine to provide sulfonamide 54. Further elaboration of this sulfonamide using the Mitsunobu procedure common to the art by treatment with an alcohol such as (2S)-2-oxiranylmethanol in the presence of triphenylphosphine and diethylazodicarboxylate provides the oxiranylsulfonamide 55. Treatment of the oxirane with sodium azide under conditions common to the art provides the azide 56 which can subsequently be treated under conditions common to the art such as triphenyl phosphine and THF-water to affect reduction to the amine 57. Standard peptide coupling conditions common to the art such as EDC and HOOBt in the presence of a base such as N-methyl-morpholine can be employed in the presence of a carboxylic acid such as N-(1-benzothien-2-ylcarbonyl)-L-leucine 4 to provide the amide product 58. Removal of the nosyl group can be accomplished by treatment with thiophenol and potassium carbonate to provide the free amine which can then be treated with an electrophilic reagent such as 2-cyanobenzene sulfonyl chloride to provide the final target 59.

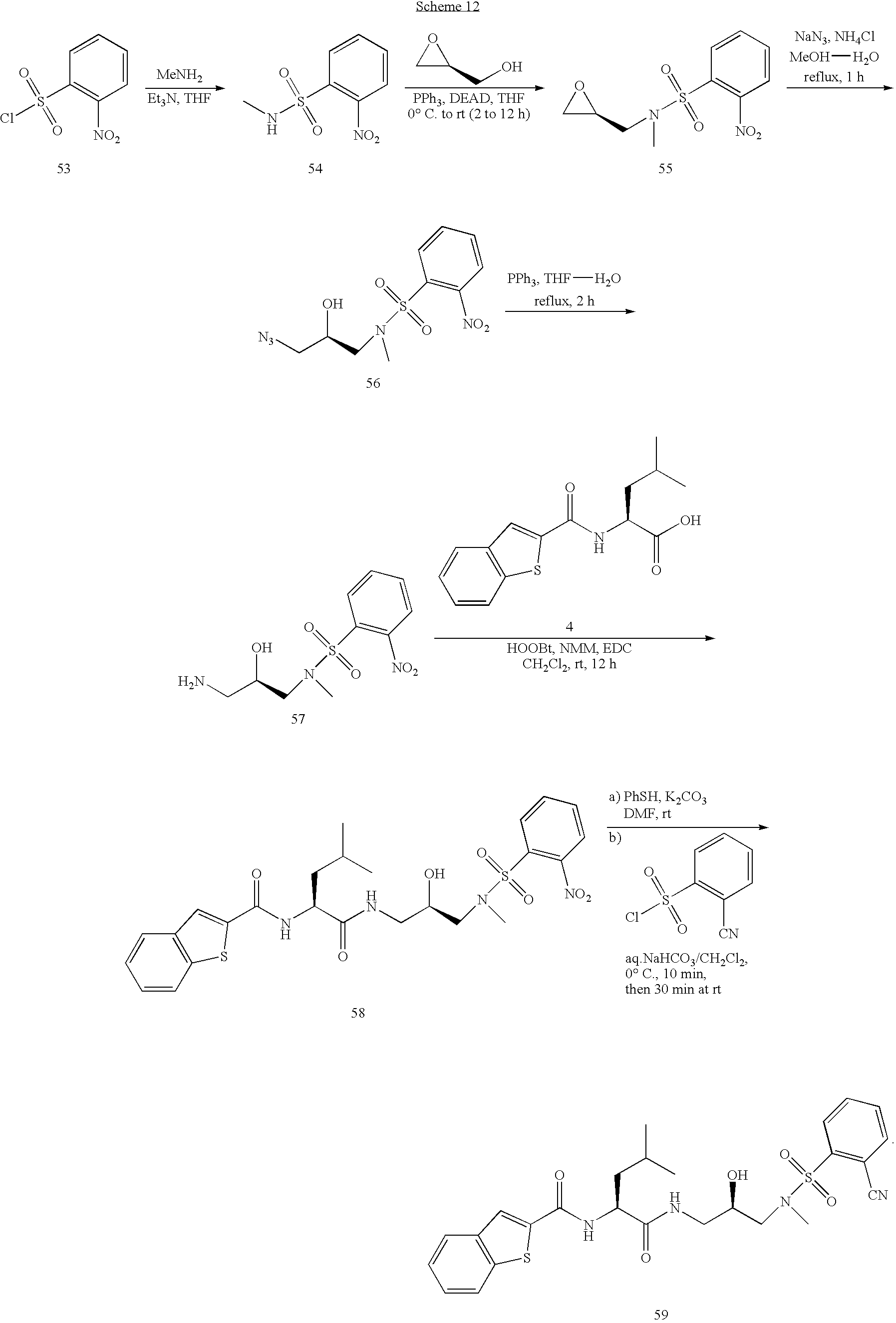

An alternative construction of the sulfonamide products is delineated in Scheme 13. 1,3-Diamino-2-propanol 60 can be treated with an electrophilic reagent such as 2,4-dichlorosulfonamide to provide sulfonamide 61. The remaining free amine can be protected under conditions common to the art by using a reagent such as Boc anhydride to provide the compound 62. Two successive alkylation steps under conditions common to the art such as iodomethane and potassium carbonate and then iodomethane and sodium hydride accomplishes methylation on the sulfonamide nitrogen and the alcohol to provide the product methyl ether 64. Following removal of the tert-butyl carbonyl group under standard conditions such as HCl or TFA to provide the free amine, standard peptide coupling conditions common to the art such as EDC and HOOBt in the presence of a base such as N-methyl-morpholine can be employed in the presence of a carboxylic acid such as N-(1-benzothien-2-ylcarbonyl)-L-leucine 4 to provide the final product 65.

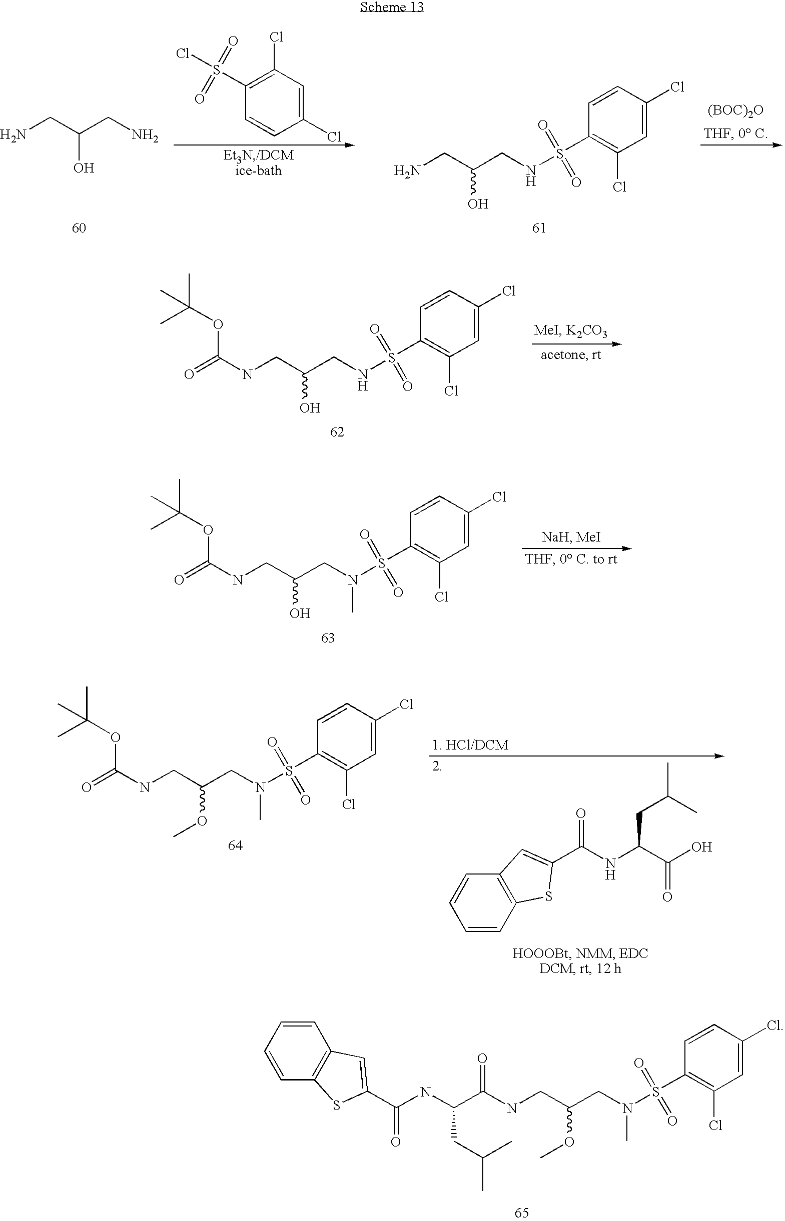

As depicted in Scheme 14, selective mono-Boc protection of the diamine 66 can be accomplished under standard conditions by treatment with 1-({[(1,1-dimethylethyl)oxy]carbonyl}oxy)-2,5-pyrrolidinedione at low temperature. Standard peptide coupling conditions common to the art such as EDC and HOOBt in the presence of a base such as N-methyl-morpholine can be employed in the presence of a carboxylic acid such as N-(1-benzothien-2-ylcarbonyl)-L-leucine 4 to provide the amide product 68. Removal of the tert-butyl carbonyl group can be accomplished using standard conditions such as HCl or TFA to provide the free amine which can then be treated with an electrophilic reagent such as 4-chlorobenzene sulfonyl chloride to provide the final target 69. Alternatively, compound 67 can be treated with an electrophilic reagent such as 2-chloro-4-fluorobenzenesulfonyl chloride to provide sulfonamide 70. Removal of the tert-butyl carbonyl group can be accomplished using standard conditions such as HCl or TFA to provide the free amine which can be coupled to a carboxylic acid such as N-(1-benzothien-2-ylcarbonyl)-L-leucine 4 under conditions common to the art such as EDC and HOOBt in the presence of a base such as N-methyl-morpholine to provide the final product 71.

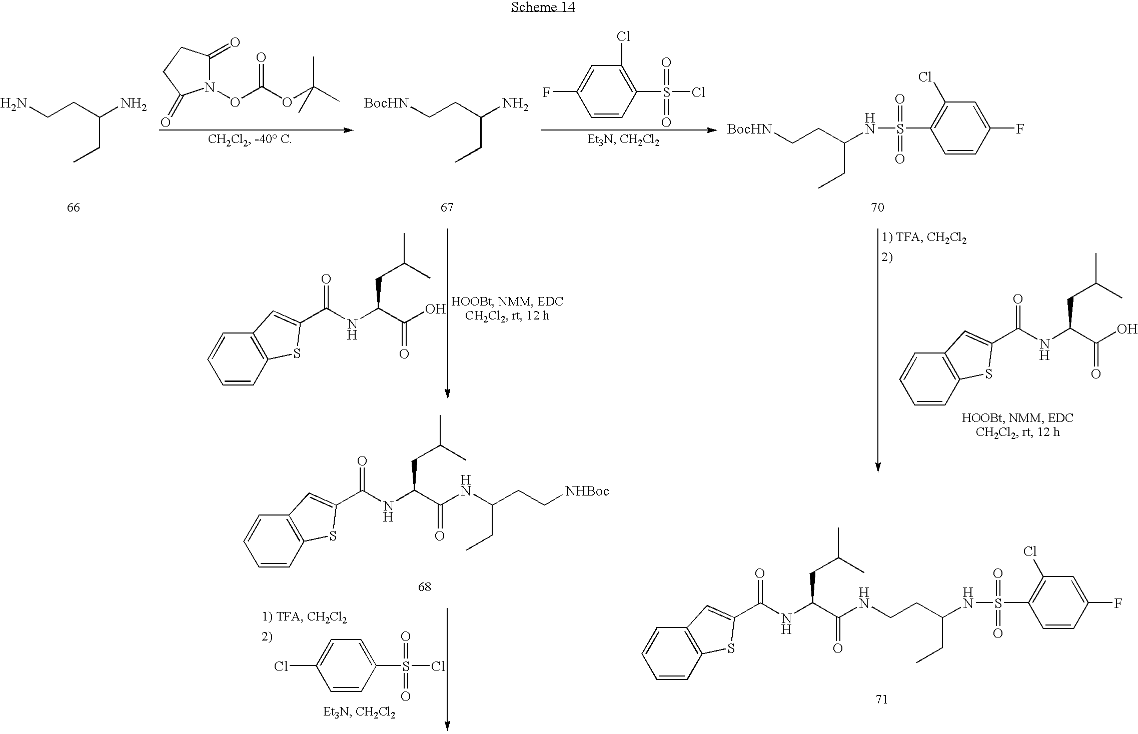

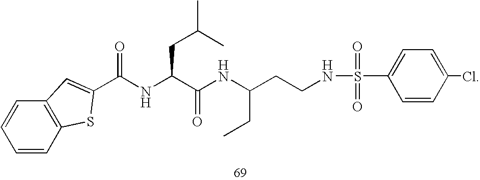

An alternative construction of the target compounds is delineated in Scheme 15. The dichlohexylamine salt of (2R)-4-({[(1,1-dimethylethyl)oxy]carbonyl}amino)-2-({[(phenylmethyl)oxy]carbonyl}amino)butanoic acid 72 can be reduced under conditions common to the art such as ethylchloroformate and sodium borohydride to provide alcohol 73. Removal of the CBZ group under standard conditions such as hydrogen and palladium on carbon and subsequent treatment of the free amine with an electrophilic reagent such as 2-chloro-4-fluorobenzenesulfonyl chloride in the presence of a base such as sodium bicarbonate provides the sulfonamide. Removal of the tert-butyl carbonyl group can be accomplished using standard conditions such as HCl or TFA to provide the free amine which can be coupled to a carboxylic acid such as N-(1-benzothien-2-ylcarbonyl)-L-leucine 4 under conditions common to the art such as EDC and HOOBt in the presence of a base such as N-methyl-morpholine to provide the final product 74. Alternatively, the hydroxymethyl intermediate 73 can be converted to the methyl diamine 76 by a two step sequence utilizing conditions common to the art involving treatment with triphenylphosphine and iodine to affect transformation to the iodomethyl compound 75 and subsequent reduction using a hydride reagent such as N-selectride at low temperature to provide the methyl compound 76. Following the same 4-step sequence delineated above provides the final compound 77.

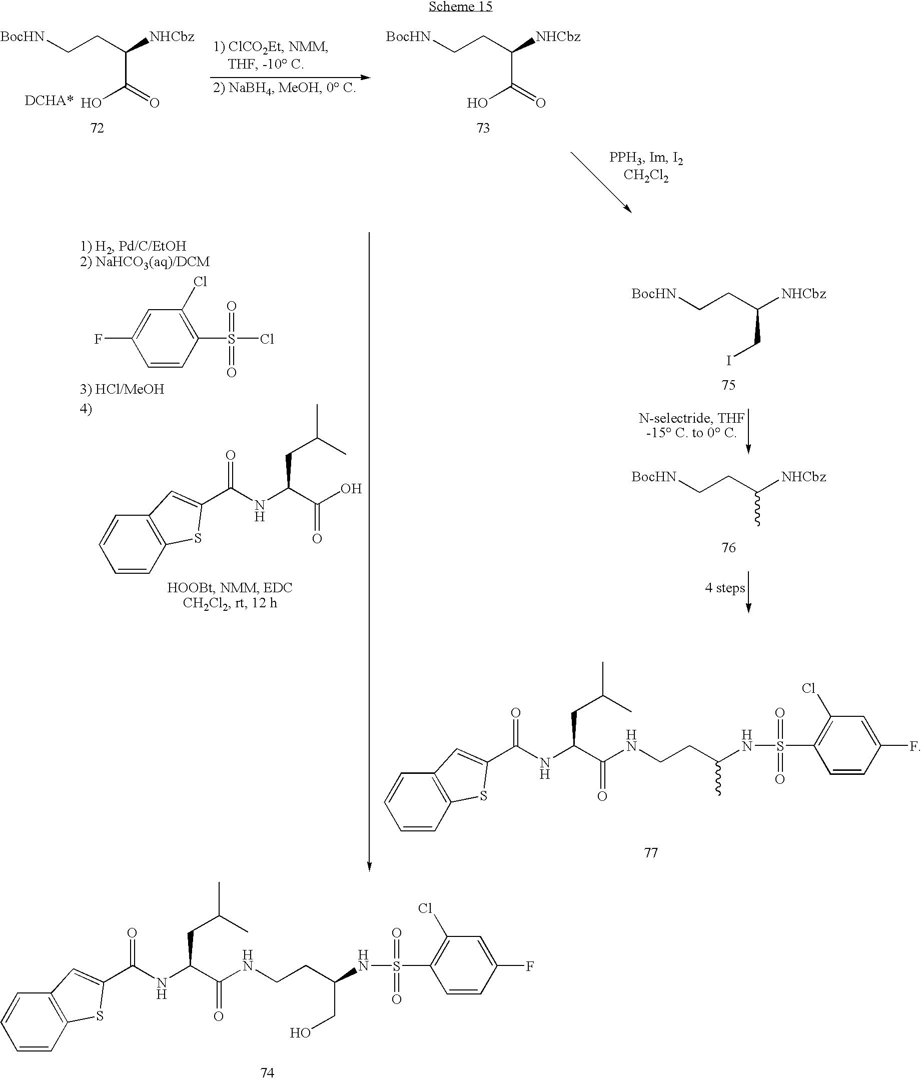

An alternative route for the construction of compound 74 is shown in Scheme 16. Treatment of methyl (2S)-2-amino-4-({[(1,1-dimethylethyl)oxy]carbonyl}amino)butanoate 78 with an electrophilic reagent such as 2-chloro-4-fluorobenzenesulfonyl chloride in the presence of a base such as sodium bicarbonate provides the sulfonamide 79. Reduction of the methyl ester under conditions common to the art such as lithium aluminum hydride at low temperature and subsequent removal of the tert-butyl carbonyl group under standard conditions such as HCl or TFA provides the free amine which can be coupled to a carboxylic acid such as N-(1-benzothien-2-ylcarbonyl)-L-leucine 4 under conditions common to the art such as EDC and HOOBt in the presence of a base such as N-methyl-morpholine to provide the final product 74.

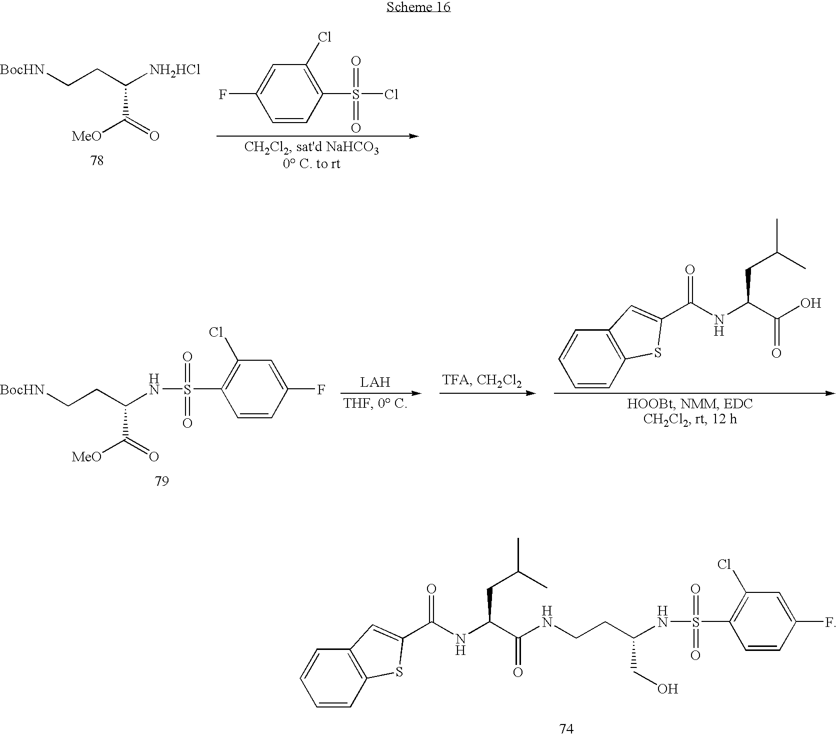

As outlined in Scheme 17, a leucine replacement analog can be prepared by treatment of 1-benzothiophene-2-carbohydrazide 75 with 2-methyl-propanal using reductive amination conditions common to the art such as sodium cyanoborohydride in the presence of an acid such as acetic acid to provide the hydrazide 76. The hydrazide can then be coupled to amine 77 under conditions common to the art such as carbonyl diimidazole to generate the final product 78.

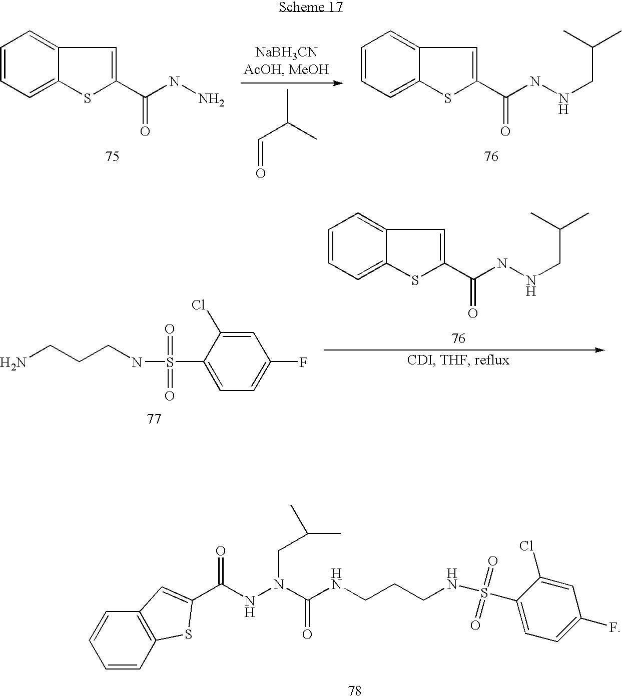

As illustrated in Scheme 18, alcohol 73 (prepared according to Scheme 15) can be treated with phthalamide according to the Mitsunobu procedure under conditions common to the art such as triphenylphosphine and diethylazodicarboxylate to provide the product 79. Removal of the tert-butyl carbonyl group under standard conditions such as HCl or TFA and subsequent coupling of the amine to a carboxylic acid such as N-(1-benzothien-2-ylcarbonyl)-L-leucine 4 under conditions common to the art such as EDC and HOOBt in the presence of a base such as N-methyl-morpholine provides the amide product 80.

Removal of the CBZ group under conditions common to the art such as hydrogen and palladium on carbon provides the free amine which can then be treated with an electrophilic reagent such as 2-chloro-4-fluorobenzene sulfonyl chloride to provide the sulfonamide 81. The phthalamido group can be removed under standard conditions by treatment with hydrazine, and subsequent treatment of the free amine with acetic anhydride in the presence of a base such as triethylamine provides the final compound 82.

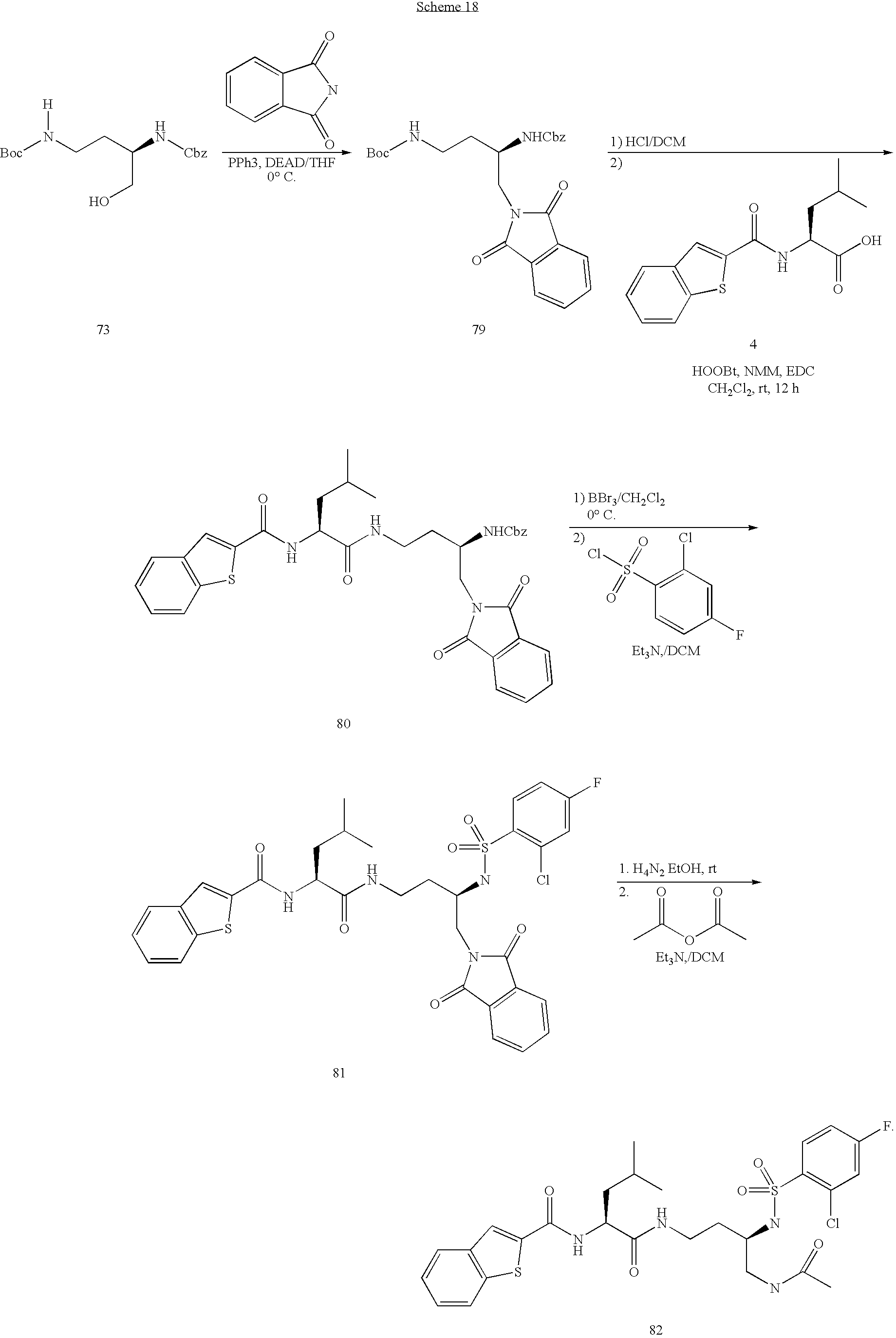

As shown in Scheme 19, treatment of (2R)-2-amino-3-buten-1-ol 83 with an electrophilic reagent such as 2-chloro-4-fluorobenzene sulfonyl chloride in the presence of a base such as triethylamine provides sulfonamide 84. Under conditions common to the art such as oxone and 1,1,1-trifluoroacetone in the presence of a base such as sodium bicarbonate, alkene 84 can be oxidized to oxirane 85. Separation of the two diastereomers provides the desired product. Treatment of the oxirane 85a with sodium azide under conditions common to the art provides the azide 86 and subsequent reduction of the azide under conditions common to the art such as hydrogen and palladium on carbon provides the amine. Standard peptide coupling conditions common to the art such as EDC and HOOBt in the presence of a base such as N-methyl-morpholine can be employed in the presence of a carboxylic acid such as N-(1-benzothien-2-ylcarbonyl)-L-leucine 4 to provide the amide product 87.

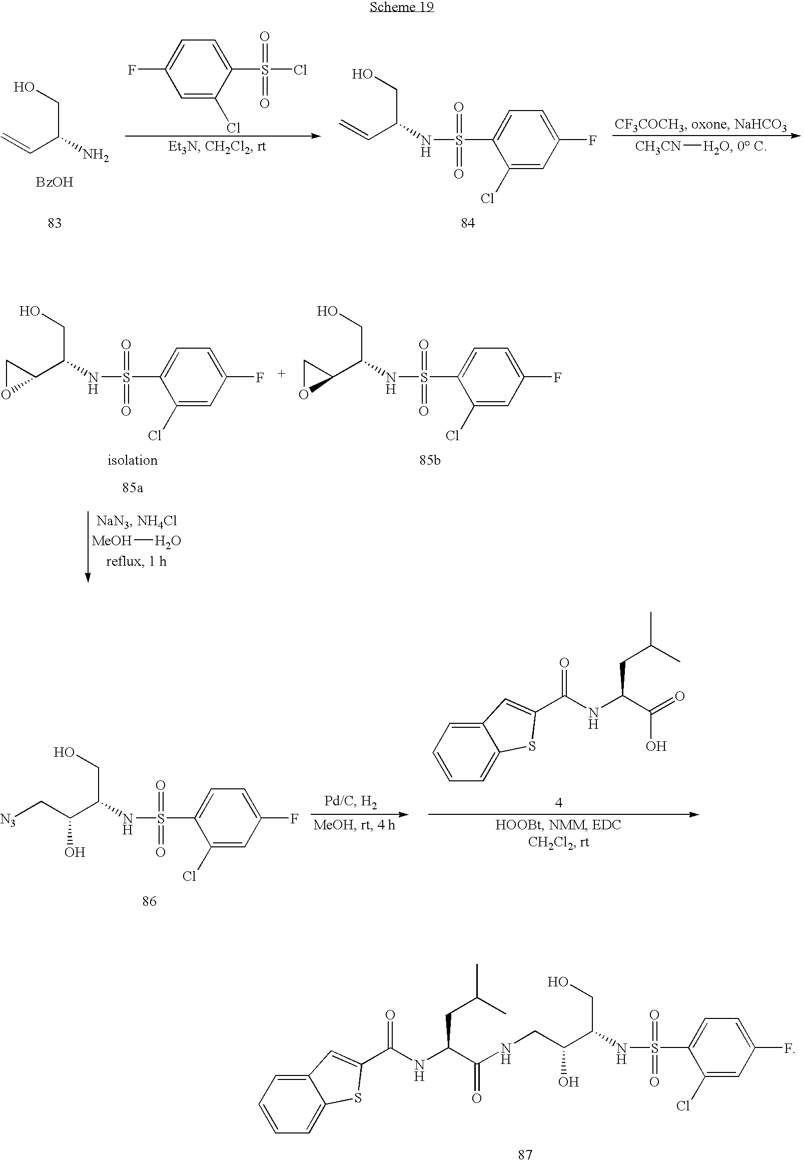

As illustrated in Scheme 20, alcohol 88 can be treated with bis(1,1-dimethylethyl) imidodicarbonate according to the Mitsunobu procedure under conditions common to the art such as triphenylphosphine and diisopropylazodicarboxylate to provide the product 89. Treatment under asymmetric dihydroxylation conditions such as potassium osmate dihydrate in the presence of methylsulfonamide followed by in situ carbamate cyclization provides the oxazolidinone 90. Conversion of the secondary alcohol to azide 91 is once again accomplished under Mitsunobu conditions common to the art such as triphenylphosphine and diethylazodicarboxylate in the presence of diphenyl phosphoryl azide. Following oxazolidinone cleavage under conditions common to the art such as cesium carbonate to provide alcohol 92, subsequent reduction of the azide using conditions common to the art such as hydrogen and palladium on carbon provides the amine compound 93. Treatment of the amine with an electrophilic reagent such as 2-chloro-4-fluorobenzenesulfonyl chloride provides sulfonamide 94. Removal of the tert-butyl carbonyl group under conditions common to the art such as HCl or TFA provides the free amine and standard peptide coupling conditions common to the art such as EDC and HOOBt in the presence of a base such as N-methyl-morpholine can be employed in the presence of a carboxylic acid such as N-(1-benzothien-2-ylcarbonyl)-L-leucine 4 to provide the amide product 95. 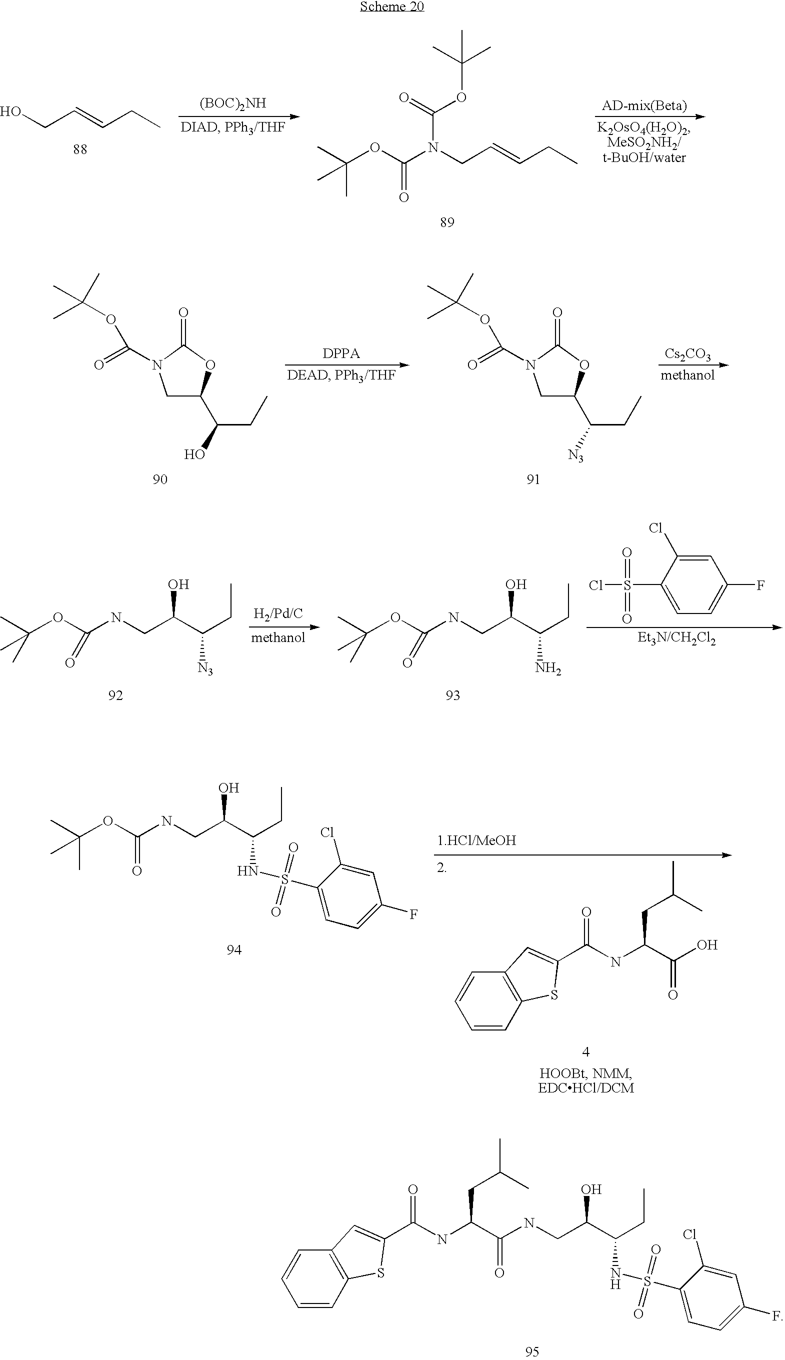
Compositions

The compounds of the invention will normally, but not necessarily, be formulated into pharmaceutical compositions prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from about 0.1 mg to about 50 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds. Conversely, the pharmaceutical compositions of the invention typically contain more than one pharmaceutically-acceptable excipient. However, in certain embodiments, the pharmaceutical compositions of the invention contain one pharmaceutically-acceptable excipient.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include, but are not limited to, the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Biological Assays

The compounds of this invention may be tested in one of several biological assays.

Ca²⁺influx mediated through TRPV4 channel receptors can be measured using articular chondrocytes from such species as, but not limited to, human, rat, canine, rabbit, monkey, and bovine, using standard techniques in the art such as, but not limited to, Fura-2 (Invitrogen/Molecular Probes, Eugene, Oreg.) fluorescence using a FlexStation (manufactured by Molecular Devices, Sunnyvale, Calif.). Table 1 lists biological data for several representative compounds obtained using this method in bovine articular chondrocytes.

TABLE 1

| Compound Example No. | EC50 values |
|---|---|
| 6 | +++ |
| 13 | ++ |
| 73 | + |

| | |
|---|---|
| Legend | |
| EC₅₀ values | |
| (in micromolar) | Symbol |
| 1-0.14 | +++ |
| 10-1.01 | ++ |
| 30-10.01 | + |

Other techniques used to measure TRPV4 channel receptor activation in chondrocytes include, but are not limited to: FLIPR assay, measuring a compound's capability to reduce the amount of ADAMTSs produced and/or released in response to a catabolic stimulus by a cell comprising a TRPV4 channel receptor; measuring a compound's capability to reduce the amount of MMPs produced and/or released in response to a catabolic stimulus by a cell comprising a TRPV4 channel receptor; measuring a compound's capability to effect the amount of nitric oxide (NO) produced in response to a catabolic stimulus by a cell comprising a TRPV4 channel receptor; and measuring a compound's capability to attenuate the inhibition of matrix synthesis in response to a catabolic stimulus by a cell comprising a TRPV4 channel receptor . Table 2 lists biological data for several representative compounds obtained using a FLIPR method.

TABLE 2

| Compound Example No. | pEC₅₀ values |
|---|---|
| 6 | +++ |
| 17 | ++ |
| 71 | + |

| | |
|---|---|
| Legend | |
| pEC₅₀ values | Symbol |
| 6.1-7.0 | +++ |
| 5.1-6.0 | ++ |
| 4.7-5.0 | + |

Legend
pEC₅₀ = −log₁₀(EC₅₀ μM)

The compounds of this invention generally show TRPV4 channel receptor modulator activity having EC₅₀ values in the range of 0.001 μM to 50 μM. The full structure/activity relationship has not yet been established for the compounds of this invention; nevertheless, one of ordinary skill in the art can readily determine which compounds of formula (I) are modulators of the TRPV4 channel receptor with an EC₅₀ value advantageously in the range of 0.001 μM to 50 μM using the assay described herein. All exemplary compounds of the present invention were assessed using at least one of the biological assays presented above. Compounds presented in the Examples had pEC₅₀ values between about 4.5 to about 7.0 as measured by Flex Station in using bovine articular cartilage and EC₅₀ values of about 0.1 μM to about 30 μM as measured by FLIPR assay using TRPV4 expressing HEK cells.

Methods of Use

The compounds of the present invention are useful as agonists of TRPV4 channel receptors and are further useful in the treatment of disease associated with TRPV4 channel receptors. Thus, the present invention further relates to a method of treating a patient in need thereof comprising administering to the patient an effective amount of a compound of formula I.

The method of the present invention may be used to treat a patient suffering from any or all of the following: a disease affecting cartilage or matrix degradation; pain, including chronic pain, neuropathic pain, and postoperative pain; osteoarthritis; neuralgia; neuropathies; algesia; nerve injury; ischaemia; neurodegeneration; cartilage degeneration; and inflammatory disorders. The method of treatment of the invention comprises administering a safe and effective amount of a compound according to Formula I or a pharmaceutically-acceptable salt thereof to the patient.

As used herein, “treatment” means: (1) the amelioration or prevention of the condition being treated or one or more of the biological manifestations of the condition being treated; (2) the interference with (a) one or more points in the biological cascade that leads to or is responsible for the condition being treated; or (b) one or more of the biological manifestations of the condition being treated, or (3) the alleviation of one or more of the symptoms or effects associated with the condition being treated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” means an amount of the compound sufficient to significantly induce a positive modification in the condition to be treated but low enough to avoid serious side effects (at a reasonable benefit/risk ratio) within the scope of sound medical judgment. A safe and effective amount of a compound of the invention will vary with the particular compound chosen; the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration range from about 0.4 to about 400 mg/kg. Typical daily dosages for parenteral administration range from about 0.01 to about 100 mg/kg; preferably between 0.1 and 20 mg/kg. The compounds of the invention may be administered alone or in combination with one or more additional active agents.

The following examples are for illustrative purposes only and are not intended to limit the scope of the invention.

### EXAMPLE 1

### Preparation of N-{(1S)-1-[({3-[[(2-chloro-4-cyanophenyl)sulfonyl](methyl)amino]propyl}amino)carbonyl]-3-methylbutyl}-1-benzothiophene-2-carboxamide

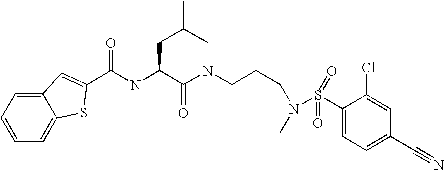 a. 1-[(1-benzothien-2-ylcarbonyl)oxy]-2,5-pyrrolidinedione

To a dichloromethane (280 ml) solution of 1-benzothiophene-2-carboxylic acid (10 g, 56.18 mmol) was added N-hydroxysuccinimide (7.11 g, 61.8 mmol) and 1-[3-(dimethylamino)propyl]-3-ethylcarbodiimide hydrochloride (EDC.HCl) (12.92 g, 67.40 mmol). The mixture was stirred overnight at room temperature. The solution was washed with brine, dried (MgSO₄), filtered and concentrated to a white solid (15.4 g) which was carried on to the next step without further purification.

MS (m/z): 276 (M+H)

b. N-(1-benzothien-2-ylcarbonyl)-L-leucine

To a solution of 1-[(1-benzothien-2-ylcarbonyl)oxy]-2,5-pyrrolidinedione (15.4 g, 56.18 mmol) in dichloromethane (85 ml), EtOH (140 ml), and deionized water (55 ml) was added L-leucine (7.66 g, 58.43 mmol). The mixture was cooled to 5-10° C. with ice-water bath, and added triethyl amine (9.4 ml, 67.42 mmol) slowly. The reaction was stirred at ambient temperature overnight. The mixture was diluted with water (50 ml) and the pH was adjusted to 1 with 6N HCl. The organic layer was washed with brine, dried (MgSO₄), filtered and concentrated to a white solid as product (16.4 g).

MS (m/z): 462 (M+H)

c. 1,1-dimethylethyl (3-{[N-(1-benzothien-2-ylcarbonyl)-L-leucyl]amino}propyl)methylcarbamate

To a dichloromethane solution of 1,1-dimethylethyl (3-aminopropyl)methylcarbamate (1.07 g, 5.67 mmol) was added N-(1-benzothien-2-ylcarbonyl)-L-leucine (1.50 g, 5.15 mmol), 3,4-dihydro-3-hydroxy-4-oxo-1,2,3-benzotriazine (HOOBT) (21 mg, 0.13 mmol), and N-methylmorpholine (NMM) (0.91 ml, 8.25 mmol). The mixture was stirred several minutes whereupon EDC.HCl (1.09 g, 5.67 mmol) was added. The reaction mixture was stirred overnight at RT. The solution was washed with 10% citric acid and brine, dried (MgSO₄), filtered and concentrated to a solid. Purification by silica gel column chromatography (30%-70% ethyl acetate/hexane) gave the product as a white solid in 85% yield (1.9 g): MS (m/z): 362 (M+H)

d. N-[(1S)-3-methyl-1-({[3-(methylamino)propyl]amino}carbonyl)butyl]-1-benzothiophene-2-carboxamide (HCl salt)

The solution of 1,1-dimethylethyl (3-{[N-(1-benzothien-2-ylcarbonyl)-L-leucyl]amino}propyl)methylcarbamate (1.2 g, 2.57 mmol) in methanol (5 ml) was treated with 4N HCl in 1,4-dioxane (5 ml, 20 mmol) and the mixture was stirred for 1 hour at room temperature. Evaporating solvent gave the product as a white solid in quantitative yield: MS (m/z): 276 (M+H)

e. N-{(1S)-1-[({3-[[(2-chloro-4-cyanophenyl)sulfonyl](methyl)amino]propyl}amino)carbonyl]-3-methylbutyl}-1-benzothiophene-2-carboxamide

To a solution of N-[(1S)-3-methyl-1-({[3-(methylamino)propyl]amino}carbonyl)butyl]-1-benzothiophene-2-carboxamide (50 mg, 0.126 mmol) in dichloromethane was added 2-chloro-4-cyanobenzenesulfonyl chloride (35.5 mg, 0.151 mmol) and triethylamine (0.1 ml, 0.630 mmol). The reaction mixture was stirred at room temperature for 4 hours and concentrated. The residue was purified by silica gel column chromatography (20%-85% EtOAc/Hexane) to give the product as a white solid in 94% yield (60 mg): MS (m/z): 561 (M+H).

### EXAMPLE 2

### Preparation of N-[(1S)-1-({[3-({[4-fluoro-2-(trifluoromethyl)phenyl]sulfonyl}amino)propyl]amino}carbonyl)-3-methylbutyl]-1-benzothiophene-2-carboxamide

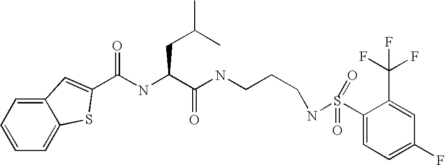

The title compound was prepared following the general procedure of Example 1 except substituting 1,1-dimethylethyl (3-aminopropyl)methylcarbamate with 1,1-dimethylethyl (3-aminopropyl)carbamate, and substituting 2-chloro-4-cyanobenzenesulfonyl chloride with 4-fluoro-2-(trifluoromethyl)benzenesulfonyl chloride: MS (m/z): 574(M+H)

### EXAMPLE 3

### Preparation of N-{(1S)-1-[({3-[[(2,4-dichloro-5-fluorophenyl)sulfonyl](methyl)amino]propyl}amino)carbonyl]-3-methylbutyl}-1-benzothiophene-2-carboxamide

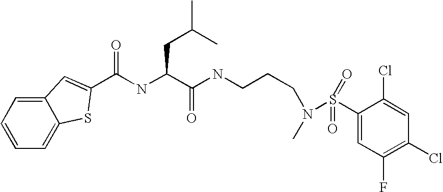

The title compound was prepared following the general procedure of Example 1 except substituting 2-chloro-4-cyanobenzenesulfonyl chloride with 2,4-dichloro-5-fluorobenzenesulfonyl chloride: MS (m/z): 588(M+H)

### EXAMPLE 4

### Preparation of N-[(1S)-1-[({3-[[(2,4-dibromophenyl)sulfonyl](methyl)amino]propyl}amino)carbonyl]-3-methylbutyl]-1-benzothiophene-2-carboxamide

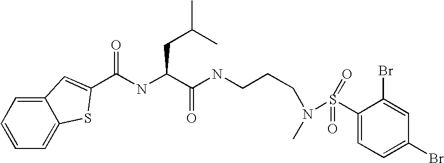

The title compound was prepared following the general procedure of Example 1 except substituting 2-chloro-4-cyanobenzenesulfonyl chloride with 2,4-dibromobenzenesulfonyl chloride: MS (m/z): 660 (M+H)

### EXAMPLE 5

### N-{(1S)-1-[({3-[[(4-bromo-2-chlorophenyl)sulfonyl](methyl)amino]propyl}amino)carbonyl]-3-methylbutyl}-1-benzothiophene-2-carboxamide

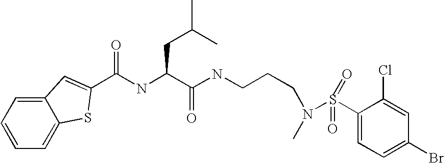

The title compound was prepared following the general procedure of Example 1 except substituting 2-chloro-4-cyanobenzenesulfonyl chloride with 2-chloro-4-bromobenzenesulfonyl chloride: MS (m/z): 616 (M+H)

### EXAMPLE 6

### Preparation of N-{(1S)-1-[({3-[[(2-chloro-4-fluorophenyl)sulfonyl](methyl)amino]propyl}amino)carbonyl]-3-methylbutyl}-1-benzothiophene-2-carboxamide

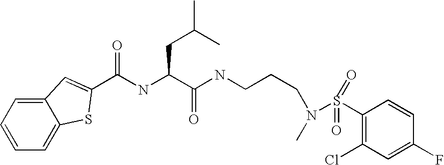

The title compound was prepared following the general procedure of Example 1 except substituting 2-chloro-4-cyanobenzenesulfonyl chloride with 2-chloro-4-fluorobenzenesulfonyl chloride: MS (m/z): 554 (M+H).

### EXAMPLE 7

### Preparation of N-{(1S)-1-[({3-[[(2,5-dichlorophenyl)sulfonyl](methyl)amino]propyl}amino)carbonyl]-3-methylbutyl}-1-benzothiophene-2-carboxamide

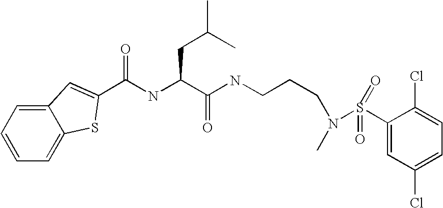

The title compound was prepared following the general procedure of Example 1 except substituting 2-chloro-4-cyanobenzenesulfonyl chloride with 2,5-dichlorobenzenesulfonyl chloride: MS (m/z): 570 (M+H).

### EXAMPLE 8

### Preparation of N-{(1S)-1-[({3-[[(2-bromo-4-fluorophenyl)sulfonyl](methyl)amino]propyl}amino)carbonyl]-3-methylbutyl}-1-benzothiophene-2-carboxamide

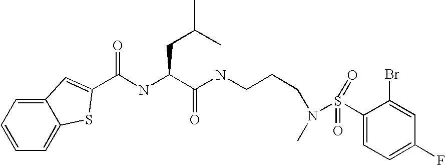

The title compound was prepared following the general procedure of Example 1 except substituting 2-chloro-4-cyanobenzenesulfonyl chloride with 2-bromo-4-fluorobenzenesulfonyl chloride: MS (m/z): 600 (M+H).

### EXAMPLE 9

### Preparation of N-{(1S)-1-[({3-[[(2-chloro-4,5-difluorophenyl)sulfonyl](methyl)amino]propyl}amino)carbonyl]-3-methylbutyl}-1-benzothiophene-2-carboxamide

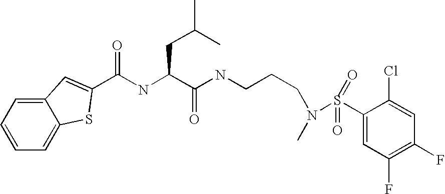

The title compound was prepared following the general procedure of Example 1 except substituting 2-chloro-4-cyanobenzenesulfonyl chloride with 2-chloro-4,5-difluorobenzenesulfonyl chloride: MS (m/z): 572 (M+H).

### EXAMPLE 10

### Preparation of N-{(1S)-1-[({3-[[(2,4-difluorophenyl)sulfonyl](methyl)amino]propyl}amino)carbonyl]-3-methylbutyl}-1-benzothiophene-2-carboxamide

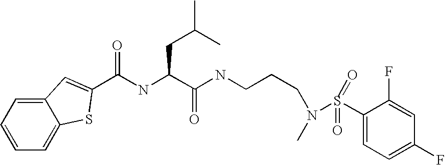

The title compound was prepared following the general procedure of Example 1 except substituting 2-chloro-4-cyanobenzenesulfonyl chloride with 2,4-difluorobenzenesulfonyl chloride: MS (m/z): 538 (M+H).

### EXAMPLE 11

### Preparation of N-{(1S)-1-[({3-[{[2-chloro-4-(trifluoromethyl)phenyl]sulfonyl}(methyl)amino]propyl}amino)carbonyl]-3-methylbutyl}-1-benzothiophene-2-carboxamide

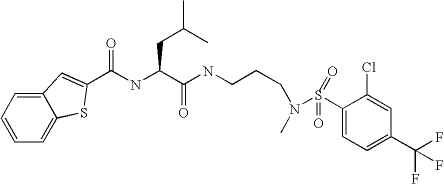

The title compound was prepared following the general procedure of Example 1 except substituting 2-chloro-4-cyanobenzenesulfonyl chloride with 2-chloro-4-(trifluoromethyl)benzenesulfonyl chloride: MS (m/z): 604 (M+H).

### EXAMPLE 12

### Preparation of N-{(1S)-1-[({3-[[(3-bromo-2-thienyl)sulfonyl](methyl)amino]propyl}amino)carbonyl]-3-methylbutyl}-1-benzothiophene-2-carboxamide

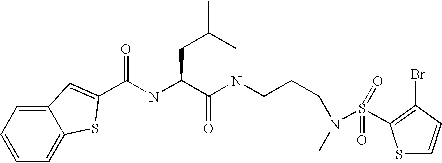 a. 3-bromo-2-thiophenesulfonyl chloride

At −78° C., to a solution of 3-bromothiophene (2.0 g, 12.3 mmol) in dichloromethane (12 ml) was added dropwise chloridosulfuric acid (4.92 ml, 73.6 mmol) over 0.5 h. The mixture was slowly allowed to attain ambient temperature and stirring continued for 4 h. The reaction was poured into ice (150 g). The aqueous layer was separated and extracted with dichloromethane twice. The organic layers were combined and washed with brine, dried (MgSO₄), filtered and concentrated. Purification by silica gel column chromatography (0%-30% ethyl acetate/hexanes) gave the product as a yellow oil in 73% yield (2.33 g): MS (m/z): 263 (M+H)

b. N-{(1S)-1-[({3-[[(3-bromo-2-thienyl)sulfonyl](methyl)amino]propyl}amino)carbonyl]-3-methylbutyl}-1-benzothiophene-2-carboxamide

The title compound was prepared following the general procedure of Example 1 except substituting 2-chloro-4-cyanobenzenesulfonyl chloride with 3-bromo-2-thiophenesulfonyl chloride: MS (m/z): 588 (M+H).

### EXAMPLE 13

### Preparation of N-{(1S)-1-[({3-[[(3-cyano-2-thienyl)sulfonyl](methyl)amino]propyl}amino)carbonyl]-3-methylbutyl}-1-benzothiophene-2-carboxamide

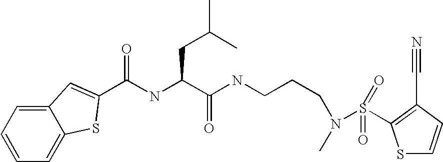

To a solution of N-{(1S)-1-[({3-[[(3-bromo-2-thienyl)sulfonyl](methyl)amino]propyl}amino)carbonyl]-3-methylbutyl}-1-benzothiophene-2-carboxamide of Example 12 (100 mg, 0.170 mmol) in DMF was added zinc cyanide (22 mg, 0.187 mmol) and tetrakis(triphenylphosphine) palladium(0) (10 mg, 0.0085 mmol). The mixture was heated in microwave for 20 minutes at 150° C. The reaction mixture was diluted with ethyl acetate and washed with water, brine, dried (MgSO₄), filtered and concentrated. Purification by silica gel column chromatography (30%-90% ethyl acetate/hexane) gave the product as a white solid in 57% yield (52 mg): MS (m/z): 533 (M+H).

### EXAMPLE 14

### Preparation of N-{(1S)-1-[({3-[[(2,6-dichlorophenyl)sulfonyl](methyl)amino]propyl}amino)carbonyl]-3-methylbutyl}-1-benzothiophene-2-carboxamide

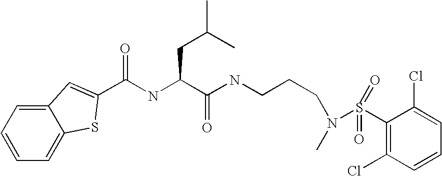

The title compound was prepared following the general procedure of Example 1 except substituting 2-chloro-4-cyanobenzenesulfonyl chloride with 2,6-dichlorobenzenesulfonyl chloride: MS (m/z): 570 (M+H).

### EXAMPLE 15

### Preparation of N-{(1S)-1-[({3-[(2,1,3-benzoxadiazol-4-ylsulfonyl)(methyl)amino]propyl}amino)carbonyl]-3-methylbutyl}-1-benzothiophene-2-carboxamide

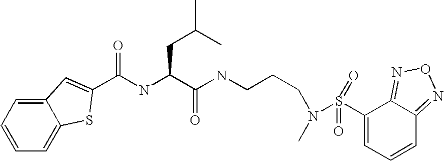

The title compound was prepared following the general procedure of Example 1 except substituting 2-chloro-4-cyanobenzenesulfonyl chloride with 2,1,3-benzoxadiazole-4-sulfonyl chloride: MS (m/z): 544 (M+H).

### EXAMPLE 16

### Preparation of N-{(1S)-1-[({3-[[(2-chloro-6-methylphenyl)sulfonyl](methyl)amino]propyl}amino)carbonyl]-3-methylbutyl}-1-benzothiophene-2-carboxamide

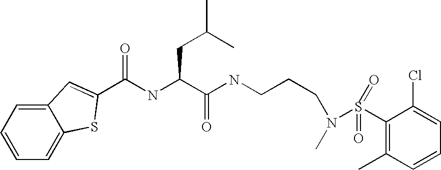

The title compound was prepared following the general procedure of Example 1 except substituting 2-chloro-4-cyanobenzenesulfonyl chloride with 2-chloro-6-methylbenzenesulfonyl chloride: MS (m/z): 550 (M+H).

### EXAMPLE 17

### Preparation of N-((1S)-3-methyl-1-{[(3-{methyl[(2,4,6-trichlorophenyl)sulfonyl]amino}propyl)amino]carbonyl}butyl)-1-benzothiophene-2-carboxamide

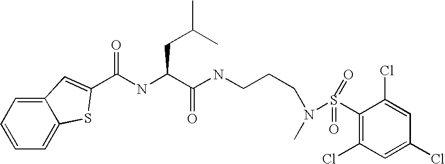

The title compound was prepared following the general procedure of Example 1 except substituting 2-chloro-4-cyanobenzenesulfonyl chloride with 2,4,6-trichlorobenzenesulfonyl chloride: MS (m/z): 606 (M+H)

### EXAMPLE 18

### Preparation of N-[(1S)-3-methyl-1-({[3-(methyl{[2-(methylsufonyl)phenyl]sulfonyl}amino)propyl]amino}carbonyl)butyl]-1-benzothiophene-2-carboxamide

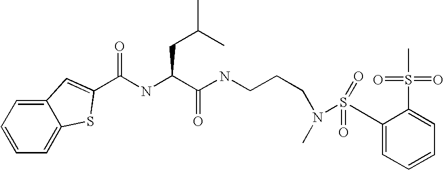

The title compound was prepared following the general procedure of Example 1 except substituting 2-chloro-4-cyanobenzenesulfonyl chloride with 2-(methylsulfonyl)benzenesulfonyl chloride: MS (m/z): 580 (M+H).

### EXAMPLE 19

### Preparation of N-{(1S)-1-[({3-[[(2-chloro-4-{[(methylamino)carbonyl]amino}phenyl)sulfonyl](methyl)amino]propyl}amino)carbonyl]-3-methylbutyl}-1-benzothiophene-2-carboxamide

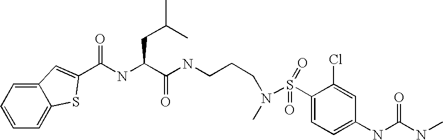

The title compound was prepared following the general procedure of Example 1 except substituting 2-chloro-4-cyanobenzenesulfonyl chloride with 2-chloro-4-{[(methylamino)carbonyl]amino}benzenesulfonyl chloride: MS (m/z): 608 (M+H).

### EXAMPLE 20

### Preparation of N-[(1S)-2-({3-[[(2-bromo-4-fluorophenyl)sulfonyl](methyl)amino]propyl}amino)-1-(cyclohexylmethyl)-2-oxoethyl]-1-benzothiophene-2-carboxamide

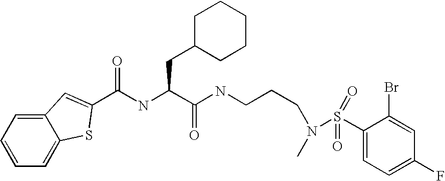 a. 3-cyclohexyl-N-{[(phenylmethyl)oxy]carbonyl}-L-alanine

To a 0° C. solution of 3-cyclohexyl-L-alanine (10 g, 58.4 mmol) in THF/H₂O (1/1, 292 ml) was added benzyl chloroformate (9.93 g, 58.4 mmol) and K₂CO₃ (9.67 g, 70.09 mmol). The mixture was stirred at 0° C. for 0.5 hour, and then stirred overnight at RT. The reaction mixture was diluted with EtOAc (200 ml) and adjusted the pH to about 2 with 6 N HCl. The organic layer was washed with brine, dried (MgSO₄), filtered and concentrated to a solid as product in 95% yield (17 g): MS (m/z): 306(M+H).

b. phenylmethyl-[(1S)-1-(cyclohexylmethyl)-2-({3-[{[(1,1-dimethylethyl)oxy]carbonyl}(methyl)amino]propyl}amino)-2-oxoethyl]carbamate

To a dichloromethane solution of 3-cyclohexyl-N-{[(phenylmethyl)oxy]carbonyl}-L-alanine (8.92 g, 29.26 mmol) was added 1,1-dimethylethyl (3-aminopropyl)carbamate (5 g g, 26.60 mmol), HOOBT (86.7 mg, 0.532 mmol), and NMM (4.4 ml, 39.9 mmol). The mixture was stirred several minutes whereupon EDC.HCl (6.12 g, 31.91 mmol) was added. The reaction mixture was stirred overnight at RT. The solution was washed with 10% citric acid and brine, dried (MgSO₄), filtered and concentrated to a solid. Purification by silica gel column chromatography (30%-90% ethyl acetate/hexane) gave the product as a white solid in 62% yield (7.8 g): MS (m/z): 476(M+H).

c. 1,1-dimethylethyl {3-[(3-cyclohexyl-L-alanyl)amino]propyl}methylcarbamate

To a solution of phenylmethyl[(1S)-1-(cyclohexylmethyl)-2-({3-[{[(1,1-dimethylethyl)oxy]carbonyl}(methyl)amino]propyl}amino)-2-oxoethyl]carbamate (5.35 g) in methanol (100 ml) was treated with 10% Pd—C (1.06 g) and the mixture was hydrogenated under hydrogen atmosphere (50 psi) at room temperature for 4 hours. Filtration and evaporation gave 3.84 g product as a colorless oil in a quantitative yield: MS (m/z): 342(M+H).

d. N-((1S)-1-(cyclohexylmethyl)-2-{[3-(methylamino)propyl]amino}-2-oxoethyl)-1-benzothiophene-2-carboxamide

To a dichloromethane solution of 1,1-dimethylethyl {3-[(3-cyclohexyl-L-alanyl)amino]propyl}methylcarbamate (3.5 g, 10.26 mmol) was added 1-benzothiophene-2-carboxylic acid (2.192 g g, 12.32 mmol), HOOBT (33 mg, 0.205 mmol), and NMM (3.38 ml, 30.78 mmol). The mixture was stirred several minutes whereupon EDC.HCl (2.362 g, 12.32 mmol) was added. The reaction mixture was stirred overnight at RT. The solution was washed with 10% citric acid and brine, dried (MgSO₄), filtered and concentrated to a solid. Purification by silica gel column chromatography (30%-90% ethyl acetate/hexane) gave the product as a white solid in 86% yield (4.4 g): MS (m/z): 502(M+H).

e. N-((1S)-1-(cyclohexylmethyl)-2-{[3-(methylamino)propyl]amino}-2-oxoethyl)-1-benzothiophene-2-carboxamide

A solution of N-((1S)-1-(cyclohexylmethyl)-2-{[3-(methylamino)propyl]amino}-2-oxoethyl)-1-benzothiophene-2-carboxamide (2.2 g, 4.39 mmol) in methanol (5 ml) was treated with 4N HCl in 1,4-dioxane (5 ml, 20 mmol) and the mixture was stirred for 2 hour at room temperature. Evaporating solvent gave the product as white solid in a quantitative yield: MS (m/z): 402(M+H).

f. N-[(1S)-2-({3-[[(2-bromo-4-fluorophenyl)sulfonyl](methyl)amino]propyl}amino)-1-(cyclohexylmethyl)-2-oxoethyl]-1-benzothiophene-2-carboxamide

To a solution of N-((1S)-1-(cyclohexylmethyl)-2-{[3-(methylamino)propyl]amino}-2-oxoethyl)-1-benzothiophene-2-carboxamide (80 mg, 0.183 mmol) in dichloromethane (1.8 ml) was added 2-bromo-4-fluorobenzenesulfonyl chloride (60 mg, 0.219 mmol) and triethylamine (0.13 ml, 0.915 mmol). The reaction mixture was stirred at room temperature for 4 hours and concentrated. The residue was purified by silica gel column chromatography (20%-85% EtOAc/Hexane) to give the product as a white solid in 62% yield (72 mg): MS (m/z): 640(M+H).

### EXAMPLE 21

### Preparation of N-[(1S)-2-({3-[[(2-chloro-4-fluorophenyl)sulfonyl](methyl)amino]propyl}amino)-1-(cyclohexylmethyl)-2-oxoethyl]-1-benzothiophene-2-carboxamide

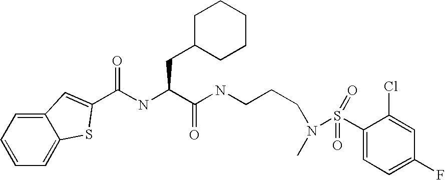

The title compound was prepared following the general procedure of Example 20 except substituting 2-bromo-4-fluorobenzenesulfonyl chloride with 2-chloro-4-fluorobenzenesulfonyl chloride: MS (m/z): 594(M+H).

### EXAMPLE 22

### Preparation of N-[(1S)-2-({3-[[(4-bromo-2-chlorophenyl)sulfonyl](methyl)amino]propyl}amino)-1-(cyclopentylmethyl)-2-oxoethyl]-1-benzothiophene-2-carboxamide

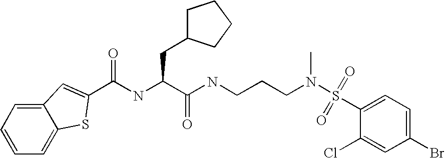

The title compound was prepared following the general procedure of Example 20 except starting with 3-cyclopentyl-L-alanine and substituting 2-bromo-4-fluorobenzenesulfonyl chloride with 2-chloro-4-bromobenzenesulfonyl chloride: MS (m/z): 641 (M+H).

### EXAMPLE 23

### Preparation of N-[(1S)-1-(cyclopentylmethyl)-2-({3-[{[4-fluoro-2-(trifluoromethyl)phenyl]sulfonyl}(methyl)amino]propyl}amino)-2-oxoethyl]-1-benzothiophene-2-carboxamide

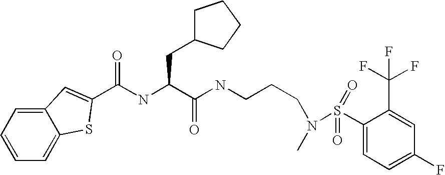

The title compound was prepared following the general procedure of Example 20 except starting with 3-cyclopentyl-L-alanine and substituting 2-bromo-4-fluorobenzenesulfonyl chloride with 4-fluoro-2-(trifluoromethyl)benzenesulfonyl chloride: MS (m/z): 614 (M+H).

### EXAMPLE 24

### Preparation of N-[(1S)-2-({3-[[(2-bromo-4-fluorophenyl)sulfonyl](methyl)amino]propyl}amino)-1-(cyclopentylmethyl)-2-oxoethyl]-1-benzothiophene-2-carboxamide

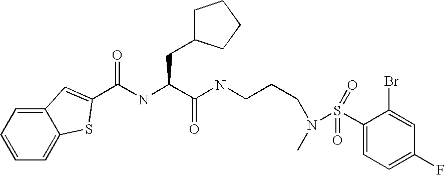

The title compound was prepared following the general procedure of Example 20 except starting with 3-cyclopentyl-L-alanine and substituting 2-bromo-4-fluorobenzenesulfonyl chloride with 2-bromo-4-fluorobenzenesulfonyl chloride: MS (m/z): 626 (M+H).

### EXAMPLE 25

### Preparation of N-[(1S)-2-({3-[[(2-chloro-4-fluorophenyl)sulfonyl](methyl)amino]propyl}amino)-1-(cyclopentylmethyl)-2-oxoethyl]-1-benzothiophene-2-carboxamide

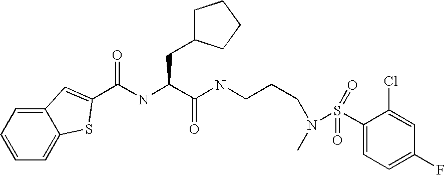

The title compound was prepared following the general procedure of Example 20 except starting with 3-cyclopentyl-L-alanine and substituting 2-bromo-4-fluorobenzenesulfonyl chloride with 2-chloro-4-fluorobenzenesulfonyl chloride: MS (m/z): 580 (M+H).

### EXAMPLE 26

### Preparation of N-[(1S)-2-({3-[[(6-chloroimidazo[2,1-b][1,3]thiazol-5-yl)sulfonyl](methyl)amino]propyl}amino)-1-(cyclopentylmethyl)-2-oxoethyl]-1-benzothiophene-2-carboxamide

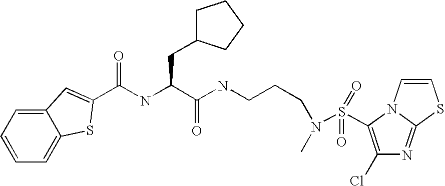

The title compound was prepared following the general procedure of Example 20 except starting with 3-cyclopentyl-L-alanine and substituting 2-bromo-4-fluorobenzenesulfonyl chloride with 6-chloroimidazo[2,1-b][1,3]thiazole-5-sulfonyl chloride: MS (m/z): 608 (M+H).

### EXAMPLE 27

### Preparation of N-[(1S)-2-({3-[[(4-chloro-2-fluorophenyl)sulfonyl](methyl)amino]propyl}amino)-1-(cyclopentylmethyl)-2-oxoethyl]-1-benzothiophene-2-carboxamide

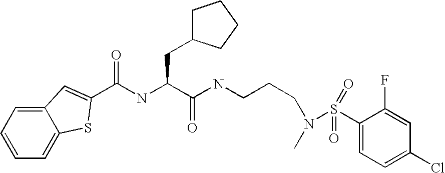

The title compound was prepared following the general procedure of Example 20 except starting with 3-cyclopentyl-L-alanine and substituting 2-bromo-4-fluorobenzenesulfonyl chloride with 4-chloro-2-fluorobenzenesulfonyl chloride: MS (m/z): 580 (M+H).

### EXAMPLE 28

### Preparation of N-[(1S)-2-({3-[[(4-bromo-2-fluorophenyl)sulfonyl](methyl)amino]propyl}amino)-1-(cyclopentylmethyl)-2-oxoethyl]-1-benzothiophene-2-carboxamide

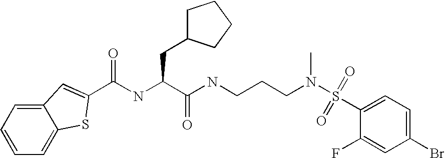

The title compound was prepared following the general procedure of Example 20 except starting with 3-cyclopentyl-L-alanine and substituting 2-bromo-4-fluorobenzenesulfonyl chloride with 4-bromo-2-fluorobenzenesulfonyl chloride: MS (m/z): 626 (M+H).

### EXAMPLE 29

### Preparation of N-[(1S)-2-({3-[[(4-cyanophenyl)sulfonyl](methyl)amino]propyl}amino)-1-(cyclopentylmethyl)-2-oxoethyl]-1-benzothiophene-2-carboxamide

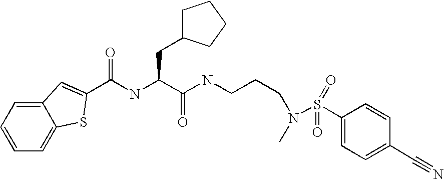

The title compound was prepared following the general procedure of Example 20 except starting with 3-cyclopentyl-L-alanine and substituting 2-bromo-4-fluorobenzenesulfonyl chloride with 4-cyanobenzenesulfonyl chloride: MS (m/z): 553 (M+H).

### EXAMPLE 30

### Preparation of N-((1S)-1-(cyclopentylmethyl)-2-{[3-(methyl{[2-(trifluoromethyl)phenyl]sulfonyl}amino)propyl]amino}-2-oxoethyl)-1-benzothiophene-2-carboxamide

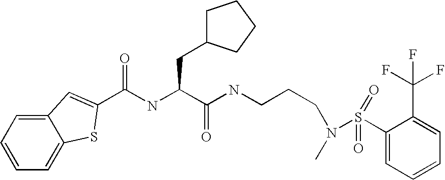

The title compound was prepared following the general procedure of Example 20 except starting with 3-cyclopentyl-L-alanine and substituting 2-bromo-4-fluorobenzenesulfonyl chloride with 2-(trifluoromethyl)benzenesulfonyl chloride: MS (m/z): 10 596 (M+H).

### EXAMPLE 31

### Preparation of N-[(1S)-2-({3-[[(4-chloro-2,5-difluorophenyl)sulfonyl](methyl)amino]propyl}amino)-1-(cyclopentylmethyl)-2-oxoethyl]-1-benzothiophene-2-carboxamide

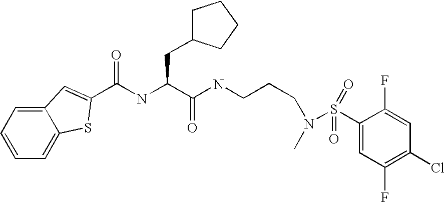

The title compound was prepared following the general procedure of Example 20 except starting with 3-cyclopentyl-L-alanine and substituting 2-bromo-4-fluorobenzenesulfonyl chloride with 4-chloro-2,5-difluorobenzenesulfonyl chloride: MS (m/z): 598 (M+H).

### EXAMPLE 32

### Preparation of N-[(1S)-1-(cyclopentylmethyl)-2-({3-[[(2,3-dichlorophenyl)sulfonyl](methyl)amino]propyl}amino)-2-oxoethyl]-1-benzothiophene-2-carboxamide

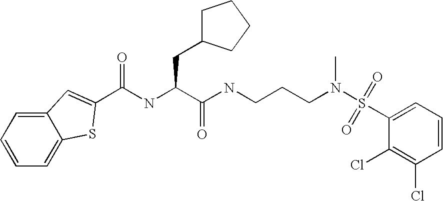

The title compound was prepared following the general procedure of Example 20 except starting with 3-cyclopentyl-L-alanine and substituting 2-bromo-4-fluorobenzenesulfonyl chloride with 2,3-dichlorobenzenesulfonyl chloride: MS (m/z): 596 (M+H).

### EXAMPLE 33

### Preparation of N-[(1S)-2-({3-[[(6-chloroimidazo[2,1-b][1,3]thiazol-5-yl)sulfonyl](methyl)amino]propyl}amino)-1-(cyclopentylmethyl)-2-oxoethyl]-1-benzothiophene-2-carboxamide

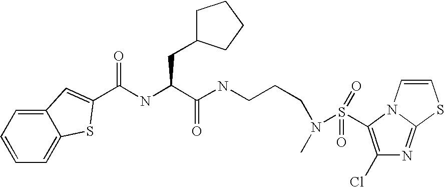

The title compound was prepared following the general procedure of Example 20 except starting with 3-cyclopentyl-L-alanine and substituting 2-bromo-4-fluorobenzenesulfonyl chloride with 6-chloroimidazo[2,1-b][1,3]thiazole-5-sulfonyl chloride: MS (m/z): 608(M+H).

### EXAMPLE 34

### Preparation of 5-bromo-N-{(1S)-1-[({3-[[(2-chloro-4-fluorophenyl)sulfonyl](methyl)amino]propyl}amino)carbonyl]-3-methylbutyl}-1-benzothiophene-2-carboxamide

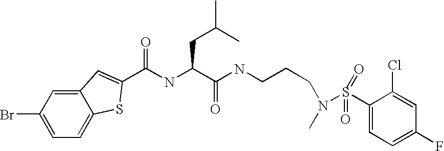 a. N¹-{3-[{[(1,1-dimethylethyl)oxy]carbonyl}(methyl)amino]propyl}-N²-{[(phenylmethyl)oxy]carbonyl}-L-leucinamide

To a dichloromethane (120 ml) solution of N-{[(phenylmethyl)oxy]carbonyl}-L-leucine (6.21 g, 23.4 mmol) was added 1,1-dimethylethyl (3-aminopropyl)methylcarbamate (4.0 g, 21.28 mmol), HOOBT (69 mg, 0.426 mmol), and NMM (3.51 ml, 31.9 mmol). The mixture was stirred several minutes whereupon EDC.HCl (4.90 g, 25.5 mmol) was added. The reaction mixture was stirred overnight at RT. The solution was washed with 10% citric acid and brine, dried (MgSO₄), filtered and concentrated to a solid. Purification by silica gel column chromatography (30%-90% ethyl acetate/hexane) gave the product as a white solid in 72% yield (7.3 g): MS (m/z): 435 (M+H).

b. N¹-[3-(methylamino)propyl]-N²-{[(phenylmethyl)oxy]carbonyl}-L-leucinamide

A solution of N¹-{3-[{[(1,1-dimethylethyl)oxy]carbonyl}(methyl)amino]propyl}-N²-{[(phenylmethyl)oxy]carbonyl}-L-leucinamide (1.3 g, 2.99 mmol) in methanol (3 ml) was treated with 4N HCl in 1,4-dioxane (2.5 ml, 10 mmol) and the mixture was stirred for 1 hour at room temperature. Evaporating solvent gave the product as a white solid in a quantitative yield: MS (m/z): 335 (M+H).

c. N¹-{3-[[(2-chloro-4-fluorophenyl)sulfonyl](methyl)amino]propyl}-N²-{[(phenylmethyl)oxy]carbonyl}-L-leucinamide

To a solution of N¹-[3-(methylamino)propyl]-N²-{[(phenylmethyl)oxy]carbonyl}-L-leucinamide (1.0 g, 2.99 mmol) in dichloromethane (15 ml) was added 2-chloro-4-fluorobenzenesulfonyl chloride (820 mg, 3.58 mmol) and triethylamine (2.1 ml, 14.9 mmol). The reaction mixture was stirred at room temperature for 4 hours and concentrated. The residue was purified by silica gel column chromatography (20%-85% EtOAc/Hexane) to give the product as a white solid in 89% yield (1.4 g). MS (m/z): 528(M+H).

d. N¹-{3-[[(2-chloro-4-fluorophenyl)sulfonyl](methyl)amino]propyl}-L-leucinamide

To a 0° C. solution of N¹-{3-[[(2-chloro-4-fluorophenyl)sulfonyl](methyl)amino]propyl}-N²-{[(phenylmethyl)oxy]carbonyl}-L-leucinamide (1.4 g, 2.66 mmol) in dichloromethane (13 ml) was added boron tribromide (1.0M solution in dichloromethane) (13.3 ml, 13.3 mmol) slowly. The mixture was stirred at 0° C. for 0.5 hour, and then stirred overnight at RT. The reaction was quenched by water (15 ml). The aqueous layer was separated, the pH was adjusted to 10 with 2 N NaOH and extracted with dichloromethane twice. The organic layers were combined and washed with brine, dried (MgSO₄), filtered and concentrated to a white solid in 81% yield (0.85 g): MS (m/z): 393(M+H).

e. 5-bromo-N-{(1S)-1-[({3-[[(2-bromo-4-fluorophenyl)sulfonyl](methyl)amino]propyl}amino)carbonyl]-3-methylbutyl}-1-benzothiophene-2-carboxamide

To a dichloromethane (1.1 mL) solution of N¹-{3-[[(2-chloro-4-fluorophenyl)sulfonyl](methyl)amino]propyl}-L-leucinamide (45.4 mg, 0.115 mmol) was added 5-bromo-1-benzothiophene-2-carboxylic acid (36 mg, 0.138 mmol), HOOBT (0.4 mg, 0.002 mmol), and NMM (0.06 ml, 0.575 mmol). The mixture was stirred several minutes whereupon EDC.HCl (26.5 mg, 0.138 mmol) was added. The reaction mixture was stirred overnight at RT. The solution was washed with 10% citric acid and brine, dried (MgSO₄), filtered and concentrated to a solid. Purification by silica gel column chromatography (30%-90% ethyl acetate/hexane) gave the product as a white solid in 70% yield (51 mg): MS (m/z): 634 (M+H).

### EXAMPLE 35

### Preparation of N-((1S)-1-{[(3-{[(2-chloro-4-fluorophenyl)sulfonyl]amino}propyl)amino]carbonyl}-3-methylbutyl)-5,6-dihydro-4H-cyclopenta[b]thiophene-2-carboxamide

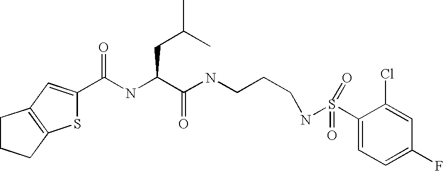

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 5,6-dihydro-4H-cyclopenta[b]thiophene-2-carboxylic acid: MS (m/z): 531 (M+H).

### EXAMPLE 36

### Preparation of 1,1-dimethylethyl 6-{[((1S)-1-{[(3-{[(2-chloro-4-fluorophenyl)sulfonyl]amino}propyl)amino]carbonyl}-3-methylbutyl)amino]carbonyl}-3,4-dihydro-2(1H)-isoquinolinecarboxylate

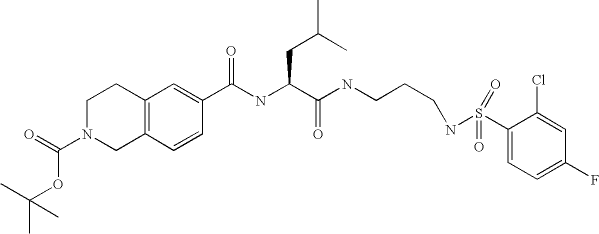

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 2-{[(1,1-dimethylethyl)oxy]carbonyl}-1,2,3,4-tetrahydro-6-isoquinolinecarboxylic acid: MS (m/z): 639 (M+H).

### EXAMPLE 37

### Preparation of 1,1-dimethylethyl 5-{[((1S)-1-{[(3-{[(2-chloro-4-fluorophenyl)sulfonyl]amino}propyl)amino]carbonyl}-3-methylbutyl)amino]carbonyl}-3,4-dihydro-2(1H)-isoquinolinecarboxylate

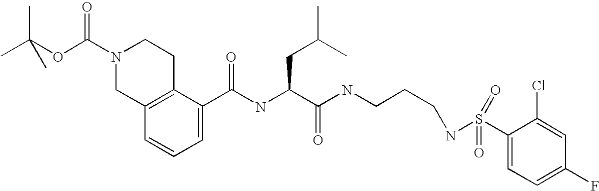

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 2-{[(1,1-dimethylethyl)oxy]carbonyl}-1,2,3,4-tetrahydro-5-isoquinolinecarboxylic acid: MS (m/z): 639 (M+H).

### EXAMPLE 38

### Preparation of N-{(1S)-1-[({3-[[(2-chloro-4-fluorophenyl)sulfonyl](methyl)amino]propyl}-amino)carbonyl]-3-methylbutyl}-4-fluoro-1-benzothiophene-2-carboxamide

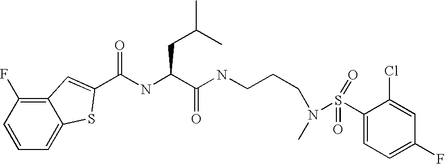

The title compound was prepared following the general procedure of Example 34 except substituting 5-bromo-1-benzothiophene-2-carboxylic acid with 4-fluoro-1-benzothiophene-2-carboxylic acid: MS (m/z): 531 (M+H).

### EXAMPLE 39

### Preparation of N¹-(3-{[(2-chloro-4-fluorophenyl)sulfonyl]amino}propyl)-N²-(5-cyclohexylpentanoyl)-leucinamide

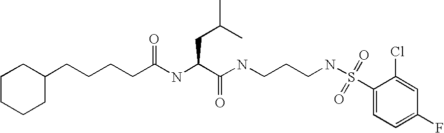

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 5-cyclohexylpentanoic acid MS (m/z): 546 (M+H).

### EXAMPLE 40

### Preparation of N¹-(3-{[(2-chloro-4-fluorophenyl)sulfonyl]amino}propyl)-N²-(4-cyclohexylbutanoyl)-L-leucinamide

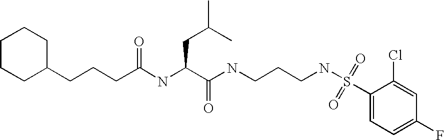

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 4-cyclohexylbutanoic acid: MS (m/z): 532 (M+H).

### EXAMPLE 41

### Preparation of 1,1-dimethylethyl 7-{[((1S)-1-{[(3-{[(2-chloro-4-fluorophenyl)sulfonyl]amino}propyl)amino]carbonyl}-3-methylbutyl)amino]carbonyl}-3,4-dihydro-2(1H)-isoquinolinecarboxylate

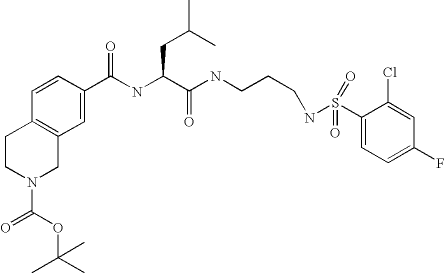

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 2-{[(1,1-dimethylethyl)oxy]carbonyl}-1,2,3,4-tetrahydro-7-isoquinolinecarboxylic acid: MS (m/z): 639 (M+H).

### EXAMPLE 42

### Preparation of N¹-(3-{[(2-chloro-4-fluorophenyl)sulfonyl]amino}propyl)-N²-({[(1-S,2S,5R)-5-methyl-2-(1-methylethyl)cyclohexyl]oxy}acetyl)-L-leucinamide

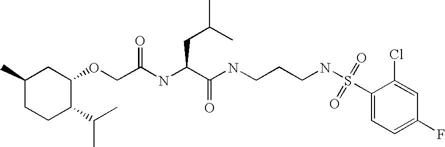

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with {[(1S,2S,5R)-5-methyl-2-(1-methylethyl)cyclohexyl]oxy}acetic acid: MS (m/z): 576 (M+H).

### EXAMPLE 43

### Preparation of N-((1S)-1-{[(3-{[(2-chloro-4-fluorophenyl)sulfonyl]amino}propyl)amino]carbonyl}-3-methylbutyl)-1-ethenyl-4,5,6,7-tetrahydro-1H-indole-2-carboxamide

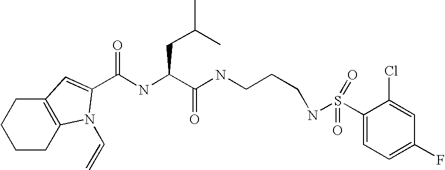

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 1-ethenyl-4,5,6,7-tetrahydro-1H-indole-2-carboxylic acid: MS (m/z): 552 (M+H).

### EXAMPLE 44

### Preparation of 3-chloro-N-{(1S)-1-[({3-[[(2-chloro-4-fluorophenyl)sulfonyl](methyl)amino]propyl}amino)carbonyl]-3-methylbutyl}-6-fluoro-1-benzothiophene-2-carboxamide

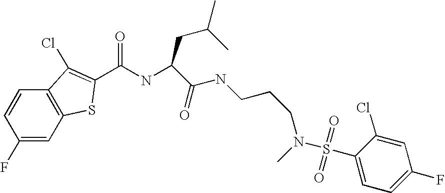

The title compound was prepared following the general procedure of Example 34 except substituting 5-bromo-1-benzothiophene-2-carboxylic acid with 3-chloro-6-fluoro-1-benzothiophene-2-carboxylic acid: MS (m/z): 606 (M+H).

### EXAMPLE 45

### Preparation of N¹-(3-{[(2-chloro-4-fluorophenyl)sulfonyl]amino}propyl)-N²-(cyclopentylacetyl)-L-leucinamide

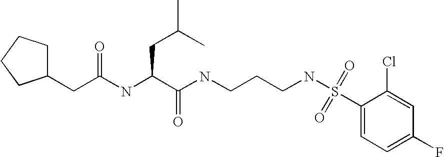

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with cyclopentylacetic acid: MS (m/z): 490 (M+H).

### EXAMPLE 46

### Preparation of N²-[(1S,4R)-bicyclo[2.2.1]hept-2-ylacetyl]-N¹-(3-{[(2-chloro-4-fluorophenyl)sulfonyl]amino}propyl)-L-leucinamide

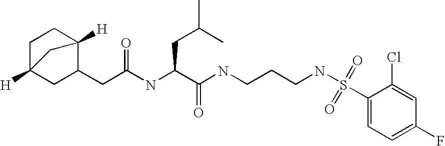

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with (1S,4R)-bicyclo[2.2.1]hept-2-ylacetic acid: MS (m/z): 516 (M+H).

### EXAMPLE 47

### Preparation of 3-chloro-N-((1S)-1-{[(3-{[(2-chloro-4-fluorophenyl)sulfonyl]amino}propyl)amino]carbonyl}-3-methylbutyl)-6-fluoro-1-benzothiophene-2-carboxamide

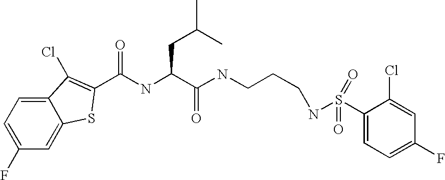

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 3-chloro-6-fluoro-1-benzothiophene-2-carboxylic acid: MS (m/z): 592 (M+H).

### EXAMPLE 48

### Preparation of 3-chloro-N-{(1S)-1-[({3-[[(2-chloro-4-fluorophenyl)sulfonyl](methyl)amino]propyl}amino)carbonyl]-3-methylbutyl}-6-methyl-1-benzothiophene-2-carboxamide

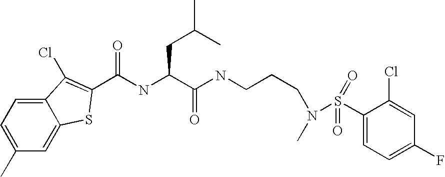

The title compound was prepared following the general procedure of Example 34 except substituting 5-bromo-1-benzothiophene-2-carboxylic acid with 3-chloro-6-methyl-1-benzothiophene-2-carboxylic acid: MS (m/z): 602 (M+H).

### EXAMPLE 49

### Preparation of 3,4-dichloro-N-{(1S)-1-[({3-[[(2-chloro-4-fluorophenyl)sulfonyl](methyl)amino]propyl}amino)carbonyl]-3-methylbutyl}-1-benzothiophene-2-carboxamide

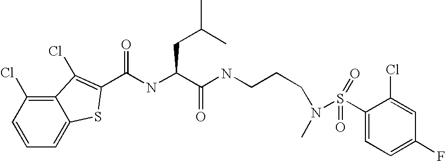

The title compound was prepared following the general procedure of Example 34 except substituting 5-bromo-1-benzothiophene-2-carboxylic acid with 3,4-dichloro-1-benzothiophene-2-carboxylic acid: MS (m/z): 624 (M+H).

### EXAMPLE 50

### Preparation of N¹-(3-{[(2-chloro-4-fluorophenyl)sulfonyl]amino}propyl)-N²-(3-phenylpropanoyl)-L-leucinamide

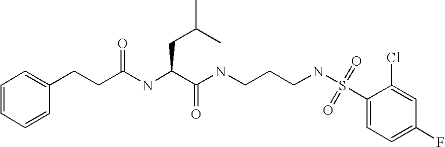

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 3-phenylpropanoic acid: MS (m/z): 512 (M+H).

### EXAMPLE 51

### Preparation of N²-(1-benzothien-2-ylacetyl)-N¹-(3-{[(2-chloro-4-fluorophenyl)sulfonyl]amino}propyl)-L-leucinamide

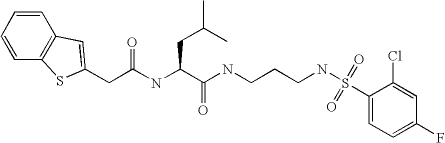

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 1-benzothien-2-ylacetic acid: MS (m/z): 553 (M+H).

### EXAMPLE 52

### Preparation of N¹-(3-{[(2-chloro-4-fluorophenyl)sulfonyl]amino}propyl)-N²-[3-(2-thienyl)propanoyl]-L-leucinamide

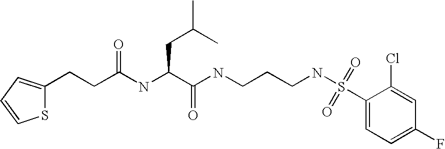

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 3-(2-thienyl)propanoic acid: MS (m/z): 538 (M+H).

### EXAMPLE 53

### Preparation of 1,1-dimethylethyl 8-{[((1S)-1-{[(3-{[(2-chloro-4-fluorophenyl)sulfonyl]amino}propyl)amino]carbonyl}-3-methylbutyl)amino]carbonyl}-3,4-dihydro-2(1H)-isoquinolinecarboxylate

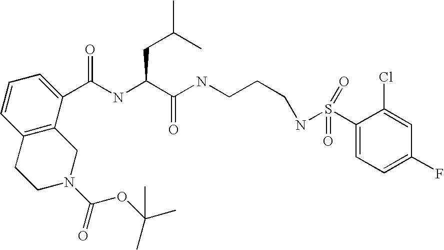

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 2-{[(1,1-dimethylethyl)oxy]carbonyl}-1,2,3,4,-tetrahydro-8-isoquinolinecarboxylic acid: MS (m/z): 639 (M+H).

### EXAMPLE 54

### Preparation of N¹-(3-{[(2-chloro-4-fluorophenyl)sulfonyl]amino}propyl)-N²-(cyclopentylcarbonyl)-L-leucinamide

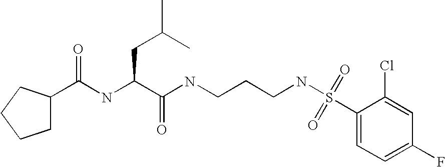

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with cyclopentanecarboxylic acid: MS (m/z): 476 (M+H).

### EXAMPLE 55

### Preparation of N¹-(3-{[(2-chloro-4-fluorophenyl)sulfonyl]amino}propyl)-N²-[(5-fluoro-1H-indol-3-yl)acetyl]-L-leucinamide

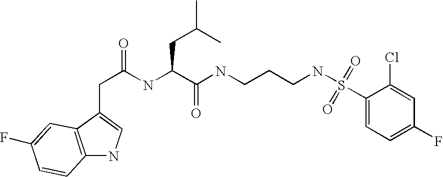

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with (5-fluoro-1H-indol-2-yl)acetic acid: MS (m/z): 555 (M+H).

### EXAMPLE 56

### Preparation of N¹-(3-{[(2-chloro-4-fluorophenyl)sulfonyl]amino}propyl)-N²-(3-cyclopropylpropanoyl)-L-leucinamide

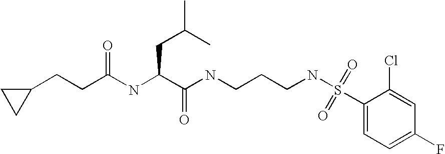

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 3-cyclopropylpropanoic acid: MS (m/z): 555 (M+H).

### EXAMPLE 57

### Preparation of N¹-(3-{[(2-chloro-4-fluorophenyl)sulfonyl]amino}propyl)-N²-[3-(1H-indol-2-yl)propanoyl]-L-leucinamide

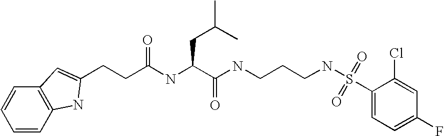

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 3-(1H-indol-2-yl)propanoic acid MS (m/z): 551 (M+H).

### EXAMPLE 58

### Preparation of N-((1S)-1-{[(3-{[(2-chloro-4-fluorophenyl)sulfonyl]amino}propyl)amino]carbonyl}-3-methylbutyl)-6-quinoxalinecarboxamide

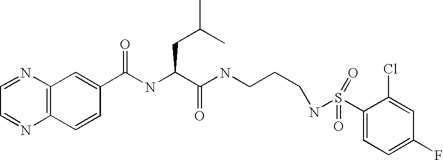

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 6-quinoxalinecarboxylic acid: MS (m/z): 536 (M+H).

### EXAMPLE 59

### Preparation of 3-amino-N-((1S)-1-{[(3-{[(2-chloro-4-fluorophenyl)sulfonyl]amino}propyl)amino]carbonyl}-3-methylbutyl)thieno[2,3-b]pyridine-2-carboxamide

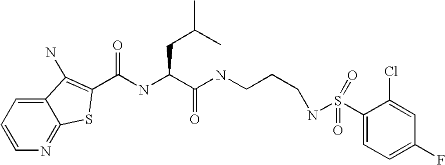

The title compound was prepared following the general procedure of Example 34 except substituting 1,1-dimethylethyl (3-aminopropyl)methylcarbamate with 1,1-dimethylethyl (3-aminopropyl)carbamate and substituting 5-bromo-1-benzothiophene-2-carboxylic acid with 3-aminothieno[2,3-b]pyridine-2-carboxylic acid: MS (m/z): 556 (M+H).

### EXAMPLE 60

### Preparation of 3-cyclopentyl-N-((1S)-2-{[3-({[4-fluoro-2-(trifluoromethyl)phenyl]sulfonyl}amino)propyl]amino}-2-oxo-1-phenylethyl)propanamide

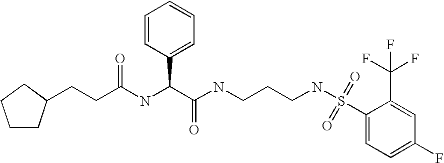 a. 1,1-dimethylethyl [3-({[4-fluoro-2-(trifluoromethyl)phenyl]sulfonyl}amino)propyl]carbamate

To a solution of 1,1-dimethylethyl (3-aminopropyl)carbamate (2.0 g, 11.4 mmol) in dichloromethane (56 ml) was added 4-fluoro-2-(trifluoromethyl)benzenesulfonyl chloride (3.61 g, 13.79 mmol) and triethylamine (4.8 ml, 34.47 mmol). The reaction mixture was stirred at room temperature for 4 hours and concentrated. The residue was purified by silica gel column chromatography (0%-50% EtOAc/Hexane) to give the product as a white solid in 75% yield (3.45 g): MS (m/z): 400(M+H).

b. N-(3-aminopropyl)-4-fluoro-2-(trifluoromethyl)benzenesulfonamide

A solution of 1,1-dimethylethyl [3-({[4-fluoro-2-(trifluoromethyl)phenyl]sulfonyl}-amino)propyl]carbamate (3.45 g, 8.62 mmol) in methanol (10 ml) was treated with 4N HCl in 1,4-dioxane (10.8 ml) and the mixture was stirred for 1 hour at room temperature. Evaporating solvent gave the product as a white solid in quantitative yield.

c. 1,1-dimethylethyl((1S)-2-{[3-({[4-fluoro-2-(trifluoromethyl)phenyl]sulfonyl}amino)propyl]amino}-2-oxo-1-phenylethyl)carbamate

To a dichloromethane solution of N-(3-aminopropyl)-4-fluoro-2-(trifluoromethyl)benzenesulfonamide (200 mg, 0.594 mmol) was added (2S)-({[(1,1-dimethylethyl)oxy]carbonyl}amino)(phenyl)ethanoic acid (179 mg, 0.713 mmol), HOOBT (1.2 mg, 0.007 mmol), and NMM (0.3 ml, 2.97 mmol). The mixture was stirred several minutes whereupon EDC.HCl (137 mg, 0.713 mmol) was added. The reaction mixture was stirred overnight at RT. The solution was washed with 10% citric acid and brine, dried (MgSO₄), filtered and concentrated to a solid. Purification by silica gel column chromatography (30%-90% ethyl acetate/hexane) gave the product in 85% yield (269 mg): MS (m/z): 533 (M+H).

d. (2S)-2-amino-N-[3-({[4-fluoro-2-(trifluoromethyl)phenyl]sulfonyl}amino)propyl]-2-phenylethanamide

A solution of 1,1-dimethylethyl ((1S)-2-{[3-({[4-fluoro-2-(trifluoromethyl)phenyl]sulfonyl}-amino)propyl]amino}-2-oxo-1-phenylethyl)carbamate (269 mg, 0.505 mmol) in methanol (2 ml) was treated with 4N HCl in 1,4-dioxane (0.6 ml), and the mixture was stirred for 1 hour at room temperature. Evaporating solvent gave the product as white solid in a quantitative yield: MS (m/z): 433 (M+H).

e. 3-cyclopentyl-N-((1S)-2-{[3-({[4-fluoro-2-(trifluoromethyl)phenyl]sulfonyl}amino)propyl]amino}-2-oxo-1-phenylethyl)propanamide

To a dichloromethane (2 ml) solution of (2S)-2-amino-N-[3-({[4-fluoro-2-(trifluoromethyl)phenyl]sulfonyl}-amino)propyl]-2-phenylethanamide (100 mg, 0.213 mmol) was added 3-cyclopentylpropanoic acid (33.3 mg, 0.234 mmol), HOOBT (0.7 mg, 0.004 mmol), and NMM (0.12 ml, 1.07 mmol). The mixture was stirred several minutes whereupon EDC.HCl (49 mg, 0.256 mmol) was added. The reaction mixture was stirred overnight at RT. The solution was washed with 10% citric acid and brine, dried (MgSO₄), filtered and concentrated to a solid. Purification by silica gel column chromatography (30%-90% ethyl acetate/hexane) gave the product as a white solid in 90% yield (110 mg): MS (m/z): 557 (M+H).

### EXAMPLE 61

### Preparation of N²-(3-cyclohexylpropanoyl)-N¹-[3-({[4-fluoro-2-(trifluoromethyl)phenyl]sulfonyl}amino)propyl]-L-leucinamide

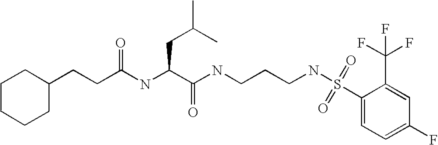

The title compound was prepared following the general procedure of Example 60 except substituting (2S)-({[(1,1-dimethylethyl)oxy]carbonyl}amino)(phenyl)ethanoic acid with N-{[(1,1-dimethylethyl)oxy]carbonyl}-L-leucine and substituting 3-cyclopentylpropanoic acid with 3-cyclohexylpropanoic acid: MS (m/z): 552 (M+H).

### EXAMPLE 62

### Preparation of N²-(3-cyclopentylpropanoyl)-N¹-[3-({[4-fluoro-2-(trifluoromethyl)phenyl]sulfonyl}amino)propyl]-L-leucinamide

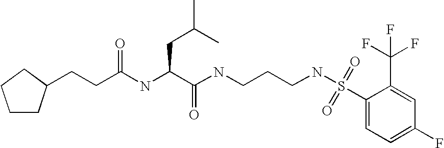

The title compound was prepared following the general procedure of Example 60 except substituting (2S)-({[(1,1-dimethylethyl)oxy]carbonyl}amino)(phenyl)ethanoic acid with N-{[(1,1-dimethylethyl)oxy]carbonyl}-L-leucine: MS (m/z): 538 (M+H).

### EXAMPLE 63

### Preparation of N-((1S)-1-{[(3-{[(2-chloro-4-fluorophenyl)sulfonyl]amino}propyl)amino]carbonyl}-3-methylbutyl)-1,2,3,4-tetrahydro-5-isoquinolinecarboxamide

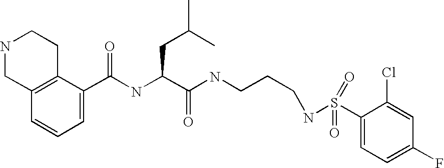

A solution of 1,1-dimethylethyl 5-{[((1S)-1-{[(3-{[(2-chloro-4-fluorophenyl)sulfonyl]amino}-propyl)amino]carbonyl}-3-methylbutyl)amino]carbonyl}-3,4-dihydro-2(1H)-isoquinolinecarboxylate from (300 mg, 0.470 mmol) in methanol (5 ml) was treated with 4N HCl in 1,4-dioxane (10 ml) and the mixture was stirred for 1 hour at room temperature. Evaporating solvent gave the product as a white solid in a quantitative yield: MS (m/z): 539 (M+H)

### EXAMPLE 64

### Preparation of N-((1S)-1-{[(3-{[(2-chloro-4-fluorophenyl)sulfonyl]amino}propyl)amino]carbonyl}-3-methylbutyl)-2-(phenylmethyl)-1,2,3,4-tetrahydro-5-isoquinolinecarboxamide

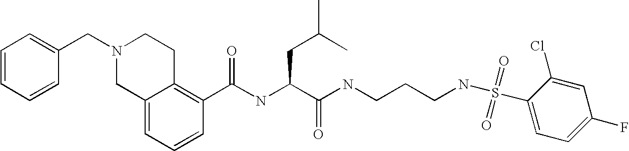

To a solution of N-((1S)-1-{[(3-{[(2-chloro-4-fluorophenyl)sulfonyl]amino}propyl)amino]carbonyl}-3-methylbutyl)-1,2,3,4-tetrahydro-5-isoquinolinecarboxamide from Example 63 (146 mg, 0.255 mmol) in dichloromethane (2.5 ml) was added (chloromethyl)benzene (39 mg, 0.306 mmol) and triethylamine (0.18 ml, 1.275 mmol). The reaction mixture was stirred at room temperature for 4 hours and concentrated. The residue was purified by silica gel column chromatography (1%-5% MeOH/CH₂Cl₂) to give the product as a white solid in 98% yield (158 mg): MS (m/z): 629(M+H).

### EXAMPLE 65

### Preparation of N-((1S)-1-{[(3-{[(2-chloro-4-fluorophenyl)sulfonyl]amino}propyl)amino]carbonyl}-3-methylbutyl)-2-(phenylcarbonyl)-1,2,3,4-tetrahydro-5-isoquinolinecarboxamide

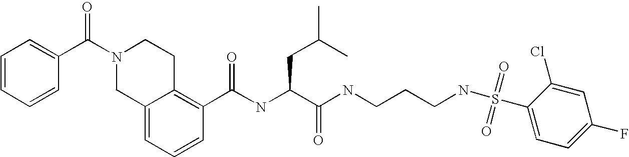

The title compound was prepared following the general procedure of Example 64 except substituting (chloromethyl)benzene with benzoyl chloride: MS (m/z): 643 (M+H).

### EXAMPLE 66

### Preparation of N-{(1S)-1-[({3-[[(cyanophenyl)sulfonyl](methyl)amino]propyl}amino)carbonyl]-3-methylbutyl}-1-benzothiophene-2-carboxamide

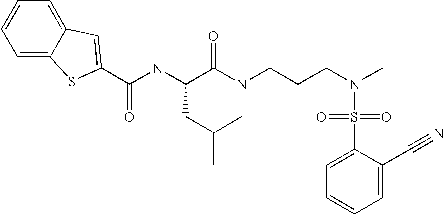 a. N-(3-aminopropyl)-2-cyano-N-methylbenzenesulfonamide

To a solution of N-methylethylene diamine (100 mg, 1.35 mmol) and 2-cyanobenzenesulfonyl chloride (250 mg, 1.23 mmol) in CH₂Cl₂ (5 ml) was added Et₃N (0.19 ml, 1.35 mmol) at 0° C. under N₂. The reaction mixture was warmed up to RT and stirred overnight. Added additional CH₂Cl₂ to the mixture, and washed the mixture with brine followed by drying over MgSO₄, filtration, and concentration under the reduced pressure. The resultant residue (226 mg) was used for the next reaction without further purification.

b. 1-[(1-benzothien-2-ylcarbonyl)oxy]-2,5-pyrrolidinedione

Charged a dry 1.0 L round bottom flask with methylene chloride (281 ml), 1-benzothiophene-2-carboxylic acid (10 g, 56.18 mmol), N-hydroxysuccinimide (7.11 g, 61.8 mmol), and 1-[3-(dimethylamino)propyl]-3-ethylcarbodiimide hydrochloride (12.92 g, 67.40 mmol), the reaction mixture was stirred under nitrogen at RT for 4 hrs. Evaporated some

CH₂Cl₂ solvent (about ½) under reduced pressure, the residue was washed by brine twice. The organic solution was dried over MgSO₄ and concentrated down. The obtained white solid product (15.4 g) was carried out to the next step without further purification.

c. N-(1-benzothien-2-ylcarbonyl)-L-leucine

Charged a dry 1.0 L round bottom flask with 1-[(1-benzothien-2-ylcarbonyl)oxy]-2,5-pyrrolidinedione (15.4 g, 56.18 mmol), L-leucine (7.66 g, 58.43 mmol), EtOH (140 ml), methylene chloride (85 ml) and deionized water (55 ml). Cooled the reaction mixture to 5-10° C. by ice-water bath, the add triethyl amine (9.4 ml, 67.42 mmol) slowly. Remove the ice water bath and the mixture was stirred at ambient temperatures overnight. Diluted the mixture with 50 ml water, and adjusted the pH to 1 with 6N HCl, extracted with methylene chloride twice. The organic solution was dried over MgSO₄ and concentrated to afford the white solid product (16.4 g).

d. N-{(1S)-1-[({3-[[(2-cyanophenyl)sulfonyl](methyl)amino]propyl}amino)carbonyl]-3-methylbutyl}-1-benzothiophene-2-carboxamide

To the solution of N-(3-aminopropyl)-2-cyano-N-methylbenzenesulfonamide (124 mg, 0.488 mmol) in CH₂Cl₂ was first added N-(1-benzothien-2-ylcarbonyl)-L-leucine (142 mg, 0.488 mmol), followed by HOOBt (2.0 mg, 0.537 mmol), cooled the mixture to 0° C. by ice-water bath, then added N-methylmorpholine (0.085 ml, 0.732 mmol). The mixture was stirred several minutes whereupon EDC.HCl (103 mg, 0.537 mmol) was added. Allowed the mixture to warm up to room temperature and kept stirring for additional 3 hrs. The reaction mixture was washed with 10% aqueous citric acid solution, saturated aq. NaHCO₃ solution, and brine. The organic layer was dried over MgSO₄ and concentrated. The residue was purified by flash column chromatography on silica gel (Biotage, 0% to 6% THF/CH₂Cl₂) to provide 133 mg of the title compound (52%); ¹H NMR (CDCl₃): δ 8.01-8.08 (d, 1H), 7.65-7.90 (m, 6H), 7.35-7.48 (m, 2H), 6.76-6.90 (m, 2H), 4.64-4.75 (m, 1H), 3.51-3.62 (m, 1H), 3.28-3.46 (m, 2H), 3.10-3.16 (m, 1H), 2.89 (s, 3H), 1.68-1.95 (m, 5H), 1.00 (d, 6H); LCMS (m/z): 527.4 [M+H]⁺.

### EXAMPLE 67

### Preparation of N-{(1S)-1-[({3-[[(2-cyanophenyl)sulfonyl](methyl)amino]propyl}amino)carbonyl]-3-methylbutyl}thieno[3,2-b]thiophene-2-carboxamide

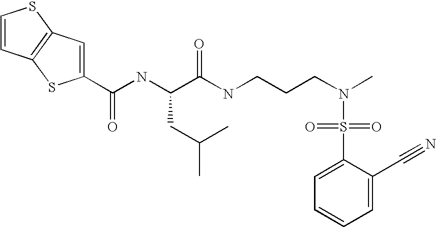

The title compound was prepared following the general procedures of Example 66 except substituting N-(1-benzothien-2-ylcarbonyl)-L-leucine (1C) for N-{[(1,1-dimethylethyl)oxy]carbonyl}-L-leucine, then followed by deprotection using 4 N HCl in dioxane to get N¹-{3-[[(2-cyanophenyl)sulfonyl](methyl)amino]propyl}-L-leucinamide (HCl salt), then couple the intermediate with thieno[3,2-b]thiophene-2-carboxylic acid by using EDC.HCl, HOOBt, and NMM: ¹H NMR (CDCl₃): δ 7.52-8.08 (m, 7H), 6.71-6.96 (m, 2H), 4.65-4.75 (m, 1H), 3.08-3.61 (m, 4H), 2.88 (s, 3H), 1.68-1.96 (m, 5H), 1.02 (d, 6H); LCMS (m/z): 533.2 [M+H]⁺.

### EXAMPLE 68

### Preparation of N-{(1S)-1-[({3-[[(2-cyanophenyl)sulfonyl](methyl)amino]propyl}amino)carbonyl]-3-methylbutyl}-1-methyl-1H-indole-2-carboxamide

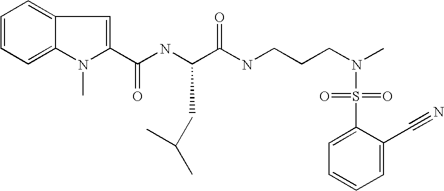

The title compound was prepared following the general procedure of Example 67 except substituting thieno[3,2-b]thiophene-2-carboxylic acid for 1-methyl-1H-indole-2-carboxylic acid: ¹H NMR (CDCl₃): δ 7.58-8.01 (m, 5H), 7.12-7.49 (m, 4H), 6.76-6.95 (m, 2H), 4.65-4.75 (m, 1H), 4.06 (s, 3H), 3.12-3.60 (m, 4H), 2.86 (s, 3H), 1.68-1.96 (m, 5H), 1.02 (d, 6H); LCMS (m/z): 524.2 [M+H]⁺.

### EXAMPLE 69

### Preparation of N-{(1S)-1-[({3-[[(2-cyanophenyl)sulfonyl](methyl)amino]propyl}amino)carbonyl]-3-methylbutyl}-1-benzofuran-2-carboxamide

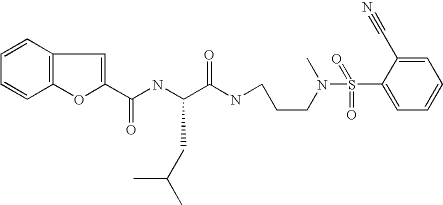

The title compound was prepared following the general procedure of Example 67 except substituting thieno[3,2-b]thiophene-2-carboxylic acid for 1-benzofuran-2-carboxylic acid: ¹H NMR (CDCl₃): δ 7.25-8.06 (m, 9H), 6.71-7.18 (m, 2H), 4.65-4.75 (m, 1H), 3.18-3.54 (m, 4H), 2.90 (s, 3H), 1.68-1.96 (m, 5H), 1.02 (d, 6H); LCMS (m/z): 538.2 [M+H]⁺.

### EXAMPLE 70

### Preparation of N²-{[2-(3-chlorophenyl)-1,3-thiazol-5-yl]carbonyl}-N¹-{3-[[(2-cyanophenyl)sulfonyl](methyl)amino]propyl}-L-leucinamide

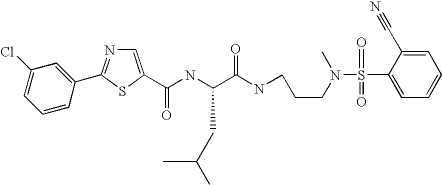

The title compound was prepared following the general procedure of Example 67 except substituting thieno[3,2-b]thiophene-2-carboxylic acid for 2-(3-chlorophenyl)-1,3-thiazole-5-carboxylic acid: ¹H NMR (CDCl₃): δ 7.28-8.15 (m, 9H), 6.71-7.85 (m, 2H), 4.65-4.75 (m, 1H), 3.15-3.54 (m, 4H), 2.90 (s, 3H), 1.68-1.98 (m, 5H), 1.02 (d, 6H); LCMS (m/z): 588.4 [M+H]⁺.

### EXAMPLE 71

### Preparation of 6-chloro-N-{(1S)-1-[({3-[[(2-cyanophenyl)sulfonyl](methyl)amino]propyl}amino)carbonyl]-3-methylbutyl}imidazo[1,2-b]pyridazine-2-carboxamide

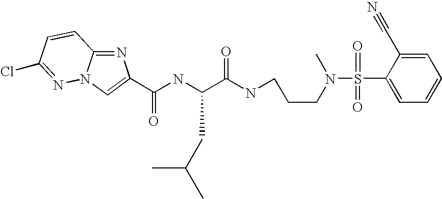

The title compound was prepared following the general procedure of Example 67 except substituting thieno[3,2-b]thiophene-2-carboxylic acid for 6-chloroimidazo[1,2-b]pyridazine-2-carboxylic acid: ¹H NMR (CDCl₃): δ 7.62-8.42 (m, 7H), 6.75-7.15 (m, 2H), 4.65-4.75 (m, 1H), 3.18-3.48 (m, 4H), 2.90 (s, 3H), 1.68-1.96 (m, 5H), 1.02 (d, 6H); LCMS (m/z): 546.1 [M+H]⁺.

### EXAMPLE 72

### Preparation of N-{(1S)-1-[({3-[[(2-cyanophenyl)sulfonyl](methyl)amino]propyl}amino)carbonyl]-3-methylbutyl}-5-methyl-1-benzothiophene-2-carboxamide

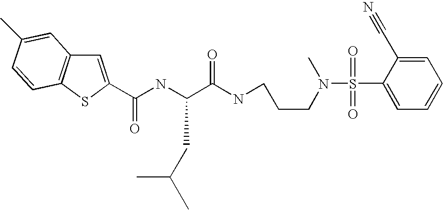

The title compound was prepared following the general procedure of Example 67 except substituting thieno[3,2-b]thiophene-2-carboxylic acid for 5-methyl-1-benzothiophene-2-carboxylic acid: ¹H NMR (CDCl₃): δ 7.12-8.06 (m, 8H), 6.71-6.92 (m, 2H), 4.65-4.72 (m, 1H), 3.12-3.54 (m, 4H), 2.88 (s, 3H), 2.48 (s, 3H), 1.68-1.96 (m, 5H), 1.02 (d, 6H); LCMS (m/z): 541.2 [M+H]⁺.

### EXAMPLE 73

### Preparation of N-{(1S)-1-[({3-[[(2-cyanophenyl)sulfonyl](methyl)amino]propyl}amino)carbonyl]-3-methylbutyl}-3-methylfuro[3,2-b]pyridine-2-carboxamide

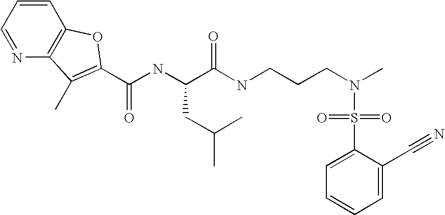

The title compound was prepared following the general procedure of Example 67 except substituting thieno[3,2-b]thiophene-2-carboxylic acid for 3-methylfuro[3,2-b]pyridine-2-carboxylic acid: ¹H NMR (CDCl₃): δ 8.64 (d, 1H), 7.35-8.08 (m, 6H), 6.75-7.15 (m, 2H), 4.65-4.72 (m, 1H), 3.22-3.52 (m, 4H), 2.89 (s, 3H), 2.66 (s, 3H), 1.68-1.96 (m, 5H), 1.02 (d, 6H); LCMS (m/z): 526.4 [M+H]⁺.

### EXAMPLE 74

### Preparation of N-[(1S)-2-({3-[[(2-cyanophenyl)sulfonyl](methyl)amino]propyl}amino)-1-(cyclopentylmethyl)-2-oxoethyl]-1-benzothiophene-2-carboxamide

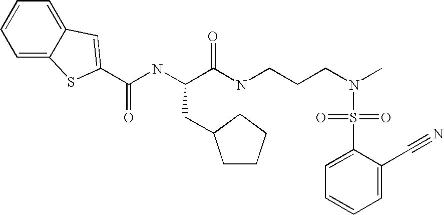

The title compound was prepared following the general procedure of Example 67 except substituting L-leucine and thieno[3,2-b]thiophene-2-carboxylic acid for 3-cyclopentyl-L-alanine and 1-benzothiophene-2-carboxylic acid: ¹H NMR (CDCl₃): δ 7.35-8.08 (m, 9H), 6.85-6.95 (m, 2H), 4.66-4.72 (m, 1H), 3.10-3.56 (m, 4H), 2.86 (s, 3H), 1.12-2.08 (m, 13H); LCMS (m/z): 553.2 [M+H]⁺.

### EXAMPLE 75

### Preparation of N-[(1S)-2-({3-[[(2-cyanophenyl)sulfonyl](methyl)amino]propyl}amino)-1-(cyclohexylmethyl)-2-oxoethyl]-1-benzothiophene-2-carboxamide

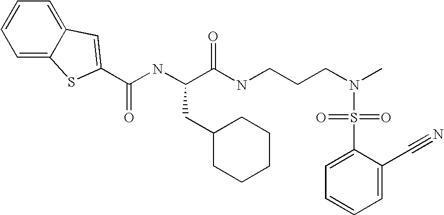

The title compound was prepared following the general procedure of Example 74 except substituting 3-cyclopentyl-L-alanine for 3-cyclohexyl-L-alanine: ¹H NMR (CDCl₃): δ 7.35-8.08 (m, 9H), 6.75-6.89 (m, 2H), 4.66-4.72 (m, 1H), 3.10-3.56 (m, 4H), 2.90 (s, 3H), 1.08-2.08 (m, 15H); LCMS (m/z): 567.3 [M+H]⁺.

### EXAMPLE 76

### Preparation of N-{(1S)-1-[({3-[[(2-cyanophenyl)sulfonyl](methyl)amino]propyl}amino)carbonyl]-3,3-dimethylbutyl}-1-benzothiophene-2-carboxamide

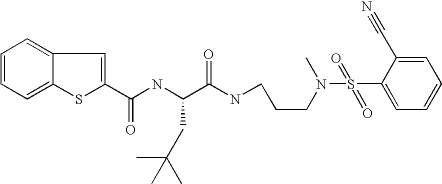

The title compound was prepared following the general procedure of Example 74 except substituting 3-cyclopentyl-L-alanine for N-{[(1,1-dimethylethyl)oxy]carbonyl}-4-methyl-L-leucine: ¹H NMR (CDCl₃): δ 7.35-8.08 (m, 9H), 6.71-6.92 (m, 2H), 4.68-4.76 (m, 1H), 3.10-3.61 (m, 4H), 2.85 (s, 3H), 1.62-2.17 (m, 5H), 1.02 (s, 9H); LCMS (m/z): 541.7 [M+H]⁺.

### EXAMPLE 77

### Preparation of N-{(1S)-1-[({3-[[(2-cyanophenyl)sulfonyl](ethyl)amino]propyl}amino)carbonyl]-3-methylbutyl}-1-benzothiophene-2-carboxamide

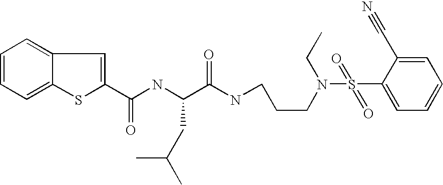 a. N²-{[(1,1-dimethylethyl)oxy]carbonyl}-N¹-[3-(ethylamino)propyl]-L-leucinamide

Diluted N-ethyl-1,3-propanediamine (150 mg, 1.468 mmol) in 15 ml of MeOH, then added the above reagent slowly to a solution of 4-nitrophenyl N-{[(1,1-dimethylethyl)oxy]carbonyl}-L-leucinate (470 mg, 1.33 mmol) in dry MeOH (20 ml) under ice-water bath. The reaction mixture was stirred at 0° C. for 2 hrs. Removed most of the solvent under reduced pressure and the residue was diluted in dichloromethane and hexane, filtered off the solid, evaporated the filtrate to provide 127 mg of the crude title compound, and used it directly for the next step without further purification.

b. N¹-{3-[[(2-cyanophenyl)sulfonyl](ethyl)amino]propyl}-N²-{[(1,1-dimethylethyl)oxy]carbonyl}-L-leucinamide

To the solution of N²-{[(1,1-dimethylethyl)oxy]carbonyl}-N¹-[3-(ethylamino)propyl]-L-leucinamide (127 mg, 0.403 mmol) in dichloromethane were added 2-cyanobenzenesulfonyl chloride (81.3 mg, 0.403 mmol) and triethylamine (0.112 ml, 0.806 mmol). This mixture was stirred at RT for 1 hr. The reaction mixture was washed by 10% aqueous citric acid, saturated aqueous NaHCO₃, and brine. The organic layer was dried over MgSO₄ and concentrated. Purification of the residue by flash column chromatography on silica gel (Biotage, 0% to 5% THF/CH₂Cl₂) provided 93 mg of the title compound (48%).

c. N-{(1S)-1-[({3-[[(2-cyanophenyl)sulfonyl](ethyl)amino]propyl}amino)carbonyl]-3-methylbutyl}-1-benzothiophene-2-carboxamide

To the compound of N¹-{3-[[(2-cyanophenyl)sulfonyl](ethyl)amino]propyl}-N²-{[(1,1-dimethylethyl)oxy]carbonyl}-L-leucinamide (93 mg, 0.194 mmol) in CH₂Cl₂/MeOH (3:1) solution was added 4M HCl in dioxane (0.484 ml, 1.94 mmol). The reaction mixture was stirred at RT for 2 hrs. Evaporated the solvent and azeotroped with toluene twice, and used it directly for the following step. To a solution of the above residue in CH₂Cl₂ (2 mL) were added 1-benzothiophene-2-carboxylic acid (34.2 mg, 0.192 mmol), HOBt (25.9 mg, 0.192 mmol), EDC.HCl (37 mg, 0.192 mmol), and triethylamine (0.054 ml, 0.384 mmol). The mixture was stirred at RT for 4 hrs. The reaction mixture was washed by 10% aqueous citric acid, saturated NaHCO₃, and brine. The organic layer was dried over MgSO₄ and concentrated. Purification of the residue by flash column chromatography on silica gel (Biotage, 0% to 6.5% THF/CH₂Cl₂) provided 60 mg of the title compound (58%); ¹H NMR (CDCl₃): δ 8.01-8.08 (d, 1H), 7.68-7.95 (m, 6H), 7.32-7.48 (m, 2H), 6.76-6.95 (m, 2H), 4.64-4.85 (m, 1H), 3.15-3.58 (m, 6H), 1.51-1.89 (m, 5H), 1.16 (t, 3H), 1.00 (d, 6H); LCMS (m/z): 541.4 [M+H]⁺.

### EXAMPLE 78

### Preparation of N-{(1S)-1-[({3-[[(2-cyanophenyl)sulfonyl](1-methylethyl)amino]propyl}amino)carbonyl]-3-methylbutyl}-1-benzothiophene-2-carboxamide

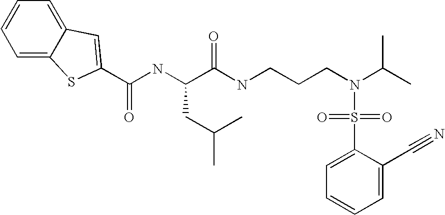

The title compound was prepared following the general procedure of Example 77 except substituting N-ethyl-1,3-propanediamine for N-(1-methylethyl)-1,3-propanediamine: ¹H NMR (CDCl₃): δ 8.06-8.08 (d, 1H), 7.68-7.85 (m, 6H), 7.36-7.45 (m, 2H), 6.76-6.95 (m, 2H), 4.66-4.83 (m, 1H), 3.18-3.62 (m, 5H), 1.49-1.91 (m, 5H), 1.18 (t, 3H), 1.08 (m, 3H), 1.00 (d, 6H); LCMS (m/z): 555.2 [M+H]⁺.

### EXAMPLE 79

### Preparation of N-{(1S)-1-[({3-[[(2-cyanophenyl)sulfonyl](propyl)amino]propyl}amino)carbonyl]-3-methylbutyl}-1-benzothiophene-2-carboxamide

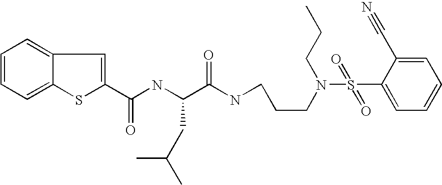

The title compound was prepared following the general procedure of Example 77 except substituting N-ethyl-1,3-propanediamine for N-propyl-1,3-propanediamine: ¹H NMR (CDCl₃): δ 7.36-8.09 (m, 9H), 6.75-6.90 (m, 2H), 4.65-4.72 (m, 1H), 3.48-3.58 (m, 2H), 3.11-3.49 (m, 4H), 1.50-1.96 (m, 7H), 1.12 (d, 6H), 0.85 (t, 3H); LCMS (m/z): 555.2 [M+H]⁺.

### EXAMPLE 80

### Preparation of N-{(1S)-1-[({3-[[(2-cyanophenyl)sulfonyl](cyclopropyl)amino]propyl}amino)carbonyl]-3-methylbutyl}-1-benzothiophene-2-carboxamide

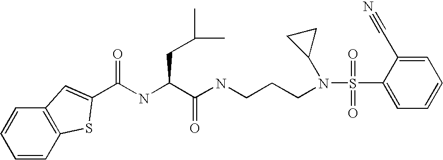 a. N¹-[3,3-bis(ethyloxy)propyl]-N²-({4-ethenyl-5-[(1Z)-1-propen-1-yl]-2-thienyl}carbonyl)-L-leucinamide

To the solution 3,3-bis(ethyloxy)-1-propanamine (379 mg, 2.576 mmol) in CH₂Cl₂ (8 mL) were added N-(1-benzothien-2-ylcarbonyl)-L-leucine (847 mg, 2.91 mmol), HOOBt (21 mg, 0.13 mmol), and N-methylmorpholine (0.37 ml, 3.35 mmol). The mixture was stirred several minutes whereupon EDC.HCl (568 mg, 2.96 mmol) was added. The reaction mixture was stirred overnight at RT. Added cold saturated aqueous NH₄Cl solution to the reaction mixture and extracted with dichloromethane twice. The combined organic layer was washed by saturated aqueous NaHCO₃ solution and brine. The organic solution was dried over MgSO₄ and concentrated. The resultant residue was purified by flash column chromatography on silica gel (Biotage, 0%-1.5% CH₂Cl₂/MeOH) to provide 0.65 g of the title compound (60%).

b. N-((1S)-3-methyl-1-{[(3-oxopropyl)amino]carbonyl}butyl)-1-benzothiophene-2-carboxamide

To the solution of N¹-[3,3-bis(ethyloxy)propyl]-N²-({4-ethenyl-5-[(1Z)-1-propen-1-yl]-2-thienyl}carbonyl)-L-leucinamide (51 mg, 0.12 mmol) in acetone/water (2 ml/0.2 ml) was added TsOH.H₂O (25 mg, 0.13 mmol). The reaction mixture was stirred at RT for 1 hr. Diluted the reaction mixture with 20 ml of H₂O and extracted with dichloromethane twice. The combined organic layer was washed by saturated NaHCO₃ solution and brine. The organic solution was dried over MgSO₄ and concentrated to provide 39 mg of the title compound (94%), which was used for the next step without further purification.

c. N-[(1S)-1-({[3-(cyclopropylamino)propyl]amino}carbonyl)-3-methylbutyl]-1-benzothiophene-2-carboxamide

To the solution of N-((1S)-3-methyl-1-{[(3-oxopropyl)amino]carbonyl}butyl)-1-benzothiophene-2-carboxamide (100 mg, 0.289 mmol) in MeOH (3 mL) were added cyclopropanamine (0.12 ml, 1.73 mmol), NaBH(OAc)₃ (18 mg, 0.289 mmol), and 4M HCl in dioxane (0.14 ml, 0.578 mmol). The reaction mixture was stirred at RT for 2 hrs. Adjusted pH to 12 by 6N aqueous NaOH, then the mixture was extracted with ethyl acetate twice. The combined organic layer was washed with brine, dried over K₂CO₃, and concentrated down to provide 58 mg of the title compound (52%), which was used for the next step without further purification.

d. N-{(1S)-1-[({3-[[(2-cyanophenyl)sulfonyl](cyclopropyl)amino]propyl}amino)carbonyl]-3-methylbutyl}-1-benzothiophene-2-carboxamide

To the solution of N-[(1S)-1-({[3-(cyclopropylamino)propyl]amino}carbonyl)-3-methylbutyl]-1-benzothiophene-2-carboxamide (44 mg, 0.11 mmol) in dichloromethane (2 mL) were added 2-cyanobenzenesulfonyl chloride (27.4 mg, 0.136 mmol) and triethylamine (0.02 ml, 0.165 mmol). The reaction mixture was concentrated and the residue was purified by flash column chromatography on silica gel (Biotage, 20%-45% EtOAc/hexane) to provide 45 mg of the title product (72%): ¹H NMR (CDCl₃): δ 7.32-8.11 (m, 9H), 6.65-6.90 (m, 2H), 4.65-4.70 (m, 1H), 3.25-3.46 (m, 4H), 2.30-2.39 (m, 1H), 1.62-1.95 (m, 5H), 1.06 (d, 6H), 0.51-0.79 (m, 4H); LCMS (m/z): 553.2 [M+H]⁺.

### EXAMPLE 81

### N-((1S)-1-{[(3{cyclopropyl[(2,4dichlorophenyl)sulfonyl]amino}propyl)amino]carbonyl}-3-methylbutyl)-1-benzothiophene-2-carboxamide

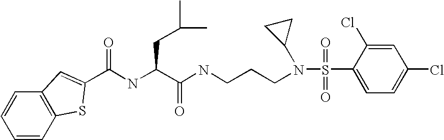

The title compound was prepared following the general procedure of Example 80 except substituting 2-cyanobenzenesulfonyl chloride for 2,4-dichlorobenzenesulfonyl chloride: ¹H NMR (CDCl₃): δ 7.32-8.09 (m, 8H), 6.62-6.90 (m, 2H), 4.65-4.70 (m, 1H), 3.42-3.51 (m, 4H), 2.39-2.48 (m, 1H), 1.62-1.95 (m, 5H), 1.02 (d, 6H), 0.45-0.62 (m, 4H); LCMS (m/z): 597.3 [M+H]⁺.

### EXAMPLE 82

### Preparation of N-{(1S)-1-[({3-[[(2-cyanophenyl)sulfonyl](tetrahydro-2H-pyran-4-yl)amino]propyl}amino)carbonyl]-3-methylbutyl}-1-benzothiophene-2-carboxamide

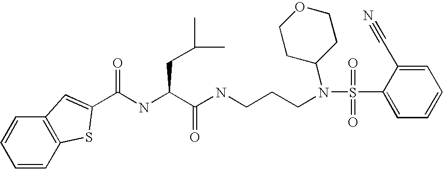

The title compound was prepared following the general procedure of Example 80 except substituting cyclopropanamine for tetrahydro-2H-pyran-4-amine: ¹H NMR (CDCl₃): δ 7.32-8.12 (m, 9H), 6.62-6.91 (m, 2H), 4.65-4.69 (m, 1H), 3.85-4.12 (m, 4H), 3.21-3.48 (m, 5H), 1.48-1.90 (m, 9H), 1.01 (d, 6H); LCMS (m/z): 596.2 [M+H]⁺.

### EXAMPLE 83

### N-{(1S)-1-[({3-[[(2-cyanophenyl)sulfonyl](2,2,2-trifluoroethyl)amino]propyl}amino)carbonyl]-3-methylbutyl}-1-benzothiophene-2-carboxamide

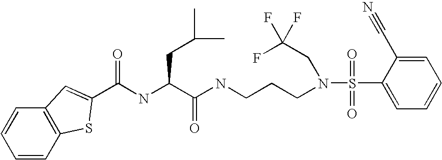

The title compound was prepared following the general procedure of Example 4 except substituting cyclopropanamine for (2,2,2-trifluoroethyl)amine: ¹H NMR (CDCl₃): δ 7.41-8.15 (m, 9H), 6.65-6.88 (m, 2H), 4.65-4.76 (m, 1H), 4.10 (q, 2H), 3.25-3.66 (m, 4H), 1.71-1.97 (m, 5H), 1.05 (d, 6H); LCMS (m/z): 595.2 [M+H]⁺.

### EXAMPLE 84

### Preparation of N-{(1S)-1-[({3-[[(2,4-dichlorophenyl)sulfonyl](methyl)amino]propyl}amino)carbonyl]-3-methylbutyl}-1-benzothiophene-2-carboxamide

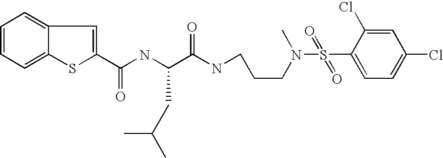 a. N-(3-aminopropyl)-N-methyl-2-nitrobenzenesulfonamide

To the solution of N-methyl-1,3-propanediamine (3.0 g, 28.8 mmol) and 2-nitrobenzenesulfonyl chloride (4.26 g, 19.2 mmol) was added triethylamine (5.35 ml, 38.4 mmol). After stirring at RT for 3 hr, pH was adjusted to 2.0-2.5 by 6N aqueous HCl, extracted with DCM twice, and then adjusted the aqueous layer to pH around 11. The aqueous solution was extracted with CHCl₃ five time. The organic layer was dried over K₂CO₃, filtered, and concentrated to get 4.6 g of yellowish oil. The crude material was used directly to next step without further purification.

b. N-((1S)-3-methyl-1-{[(3-{methyl[(2-nitrophenyl)sulfonyl]amino}propyl)amino]carbonyl}butyl)-1-benzothiophene-2-carboxamide

To the solution of N-(3-aminopropyl)-N-methyl-2-nitrobenzenesulfonamide (1.84 g, 6.74 mmol) in CH₂Cl₂ were first N-(1-benzothien-2-ylcarbonyl)-L-leucine (1.96 g, 6.74 mmol) and HOOBt (27.5 mg, 0.168 mmol). After cooling the mixture to 0° C. by ice-water bath, N-methylmorpholine (1.48 ml, 13.46 mmol) was added. The mixture was stirred several minutes whereupon EDC.HCl (1.29 g, 6.73 mmol) was added. Allowed the reaction mixture to warm up to room temperature and kept stirring for additional 3 hr. The reaction mixture was washed with 10% aqueous citric acid solution, saturated aqueous NaHCO₃ solution, and brine. The organic solution was dried over MgSO₄ followed by filtration and concentration. The residue was purified by flash column chromatography (Biotage, 0%-10% THF/CH₂Cl₂) to provide 1.49 g of the title compound (41%).

c. N-[(1S)-3-methyl-1-({[3-(methylamino)propyl]amino}carbonyl)butyl]-1-benzothiophene-2-carboxamide

To a solution of N-((1S)-3-methyl-1-{[(3-{methyl[(2-nitrophenyl)sulfonyl]amino}propyl)amino]carbonyl}butyl)-1-benzothiophene-2-carboxamide (834 mg, 1.53 mmol) in DMF (5 mL) were added benzenethiol (0.235 ml, 2.29 mmol) and K₂CO₃ (633 mg, 4.58 mmol). The reaction mixture was stirred at RT for 4 hr. Solvent were removed and the residue was diluted by 3 ml of water. The reaction mixture was acidified to pH 1.5 with 1N HCl followed by extraction with CH₂Cl₂, and then washed the organic layer with 1N HCl five times. The combined aqueous solution was basified to pH 12.5 with 6N NaOH. After extraction with ethyl acetate three times, the organic combined organic layer was washed with saturated aqueous NaHCO₃ and brine. The organic layer dried over MgSO₄, filtered, concentrated by rotary evaporation to give 420 mg of the title compound, which was used for the next step without further purification.

d. N-{(1S)-1-[({3-[[(2,4-dichlorophenyl)sulfonyl](methyl)amino]propyl}amino)carbonyl]-3-methylbutyl}-1-benzothiophene-2-carboxamide

To a solution of N-[(1S)-3-methyl-1-({[3-(methylamino)propyl]amino}carbonyl)butyl]-1-benzothiophene-2-carboxamide (50 mg, 0.139 mmol) in CH₂Cl₂ (1.5 mL) were added triethylamine (0.042 ml, 0.305 mmol) and 2,4-dichlorobenzenesulfonyl chloride (37.3 mg, 0.152 mmol). After stirring at RT for 2 hr, the reaction mixture was washed by 10% aqueous citric acid, saturated NaHCO₃, and brine. The organic layer was dried over MgSO₄ and concentrated. Purification of the residue by flash column chromatography on silica gel (Biotage, 0% to 8.0% THF/ CH₂Cl₂) provided 71 mg of the title compound (89%); ¹H NMR (CDCl₃): δ 7.21-7.98 (m, 8H), 6.72-6.89 (m, 1H), 6.62-6.68 (m, 1H), 4.65-4.75 (m, 1H), 3.25-3.46 (m, 4H), 2.86 (s, 3H), 1.68-1.95 (m, 5H), 1.06 (d, 6H); LCMS (m/z): 571.2 [M+H]⁺.

### EXAMPLE 85

### Preparation of N-{(1S)-3-methyl-1-[({3-[methyl(2-thienylsulfonyl)amino]propyl}amino)carbonyl]butyl}-1-benzothiophene-2-carboxamide

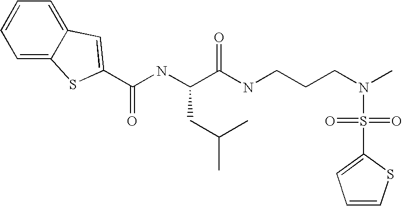

The title compound was prepared following the general procedure of Example 84 except substituting 2,4-dichlorobenzenesulfonyl chloride for 2-thiophenesulfonyl chloride: %); ¹H NMR (CDCl₃): δ 7.08-7.88 (m, 8H), 6.72-6.88 (m, 2H), 4.65-4.72 (m, 1H), 3.02-3.50 (m, 4H), 2.75 (s, 3H), 1.69-1.90 (m, 5H), 1.01 (d, 6H); LCMS (m/z): 508.7 [M+H]⁺.

### EXAMPLE 86

### Preparation of N-{(1S)-1-[({3-[[(4-bromo-3-thienyl)sulfonyl](methyl)amino]propyl}amino)carbonyl]-3-methylbutyl}-1-benzothiophene-2-carboxamide

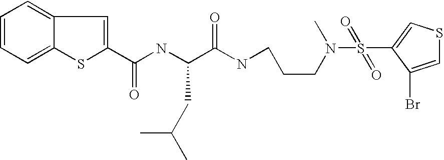

The title compound was prepared following the general procedure of Example 84 except substituting 2,4-dichlorobenzenesulfonyl chloride for 4-bromo-3-thiophenesulfonyl chloride: ¹H NMR (CDCl₃): δ 7.11-7.92 (m, 7H), 6.72-6.81 (m, 2H), 4.65-4.75 (m, 1H), 3.22-3.52 (m, 4H), 2.95 (s, 3H), 1.69-1.94 (m, 5H), 1.02 (d, 6H); LCMS (m/z): 587.4 [M+H]⁺.

### EXAMPLE 87

### Preparation of N-{(1S)-1-[({3-[[(3-bromo-5-chloro-2-thienyl)sulfonyl](methyl)amino]propyl}amino)carbonyl]-3-methylbutyl}-1-benzothiophene-2-carboxamide

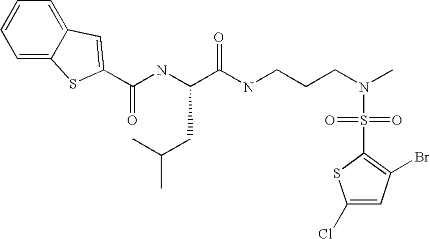

The title compound was prepared following the general procedure of Example 84 except substituting 2,4-dichlorobenzenesulfonyl chloride for 3-bromo-5-chloro-2-thiophenesulfonyl chloride: ¹H NMR (CDCl₃): δ 6.95-7.90 (m, 6H), 6.60-6.78 (m, 2H), 4.65-4.70 (m, 1H), 3.26-3.50 (m, 4H), 2.95 (s, 3H), 1.62-1.90 (m, 5H), 1.03(d, 6H); LCMS (m/z): 622.0 [M+H]⁺.

### EXAMPLE 88

### Preparation of Methyl 4-{[(3-{[N-(1-benzothien-2-ylcarbonyl)-L-leucyl]amino}propyl)(methyl)amino]sulfonyl}-2,5-dimethyl-3-furancarboxylate

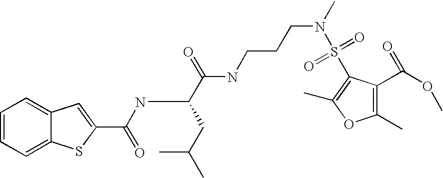

The title compound was prepared following the general procedure of Example 84 except substituting 2,4-dichlorobenzenesulfonyl chloride for methyl 4-(chlorosulfonyl)-2,5-dimethyl-3-furancarboxylate: ¹H NMR (CDCl₃): δ 7.39-7.78 (m, 5H), 6.73-7.02 (m, 2H), 4.72-4.82 (m, 1H), 3.85 (s, 3H), 3.21-3.60 (m, 4H), 2.85 (s, 3H), 2.61 (s, 3H), 2.50 (s, 3H), 1.69-1.90 (m, 5H), 1.02 (d, 6H); LCMS (m/z): 578.6 [M+H]⁺.

### EXAMPLE 89

### Preparation of N-{(1S)-1-[({3-[[(4-chlorophenyl)sulfonyl](methyl)amino]propyl}amino)carbonyl]-3-methylbutyl}-1-benzothiophene-2-carboxamide

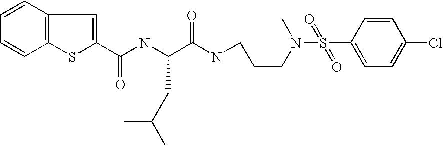

The title compound was prepared following the general procedure of Example 84 except substituting 2,4-dichlorobenzenesulfonyl chloride for 4-chlorobenzenesulfonyl chloride: ¹H NMR (CDCl₃): δ 7.36-7.92 (m, 9H), 6.61-6.94 (m, 2H), 4.65-4.72 (m, 1H), 2.95-3.51 (m, 4H), 2.73 (s, 3H), 1.68-1.96 (m, 5H), 1.02 (d, 6H); LCMS (m/z): 536.2 [M+H]⁺.

### EXAMPLE 90

### Preparation of N-{(1S)-1-[({3-[[(2-chlorophenyl)sulfonyl](methyl)amino]propyl}amino)carbonyl]-3-methylbutyl}-1-benzothiophene-2-carboxamide

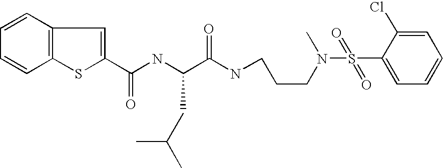

The title compound was prepared following the general procedure of Example 84 except substituting 2-cyanobenzenesulfonyl chloride for 2-chlorobenzenesulfonyl chloride: ¹H NMR (CDCl₃): δ 7.36-8.06 (m, 9H), 6.69-6.91 (m, 2H), 4.65-4.76 (m, 1H), 3.29-3.51 (m, 4H), 2.90 (s, 3H), 1.68-1.96 (m, 5H), 1.07 (d, 6H); LCMS (m/z): 536.2 [M+H]⁺.

### EXAMPLE 91

### Preparation of N-{(1S)-1-[({3-[[(2-bromophenyl)sulfonyl](methyl)amino]propyl}amino)carbonyl]-3-methylbutyl}-1-benzothiophene-2-carboxamide

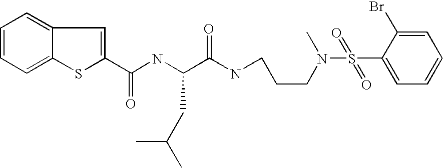

The title compound was prepared following the general procedure of Example 84 except substituting 2-cyanobenzenesulfonyl chloride for 2-bromobenzenesulfonyl chloride: ¹H NMR (CDCl₃): δ 7.36-8.05 (m, 9H), 6.65-6.92 (m, 2H), 4.65-4.72 (m, 1H), 3.30-3.51 (m, 4H), 2.89 (s, 3H), 1.62-1.96 (m, 5H), 1.06 (d, 6H); LCMS (m/z): 582.2 [M+H]⁺.

### EXAMPLE 92

### Preparation of N-((1S)-3-methyl-1-{[(3-{methyl[(2-nitrophenyl)sulfonyl]amino}propyl)amino]carbonyl}butyl)-1-benzothiophene-2-carboxamide

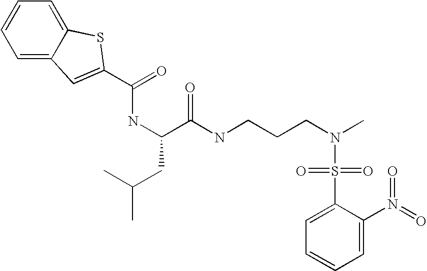

The title compound was prepared as Example 84b: ¹H NMR (CDCl₃): δ 7.36-8.08 (m, 9H), 6.61-7.04 (m, 2H), 4.65-4.72 (m, 1H), 3.18-3.51 (m, 4H), 2.98 (s, 3H), 1.68-2.02 (m, 5H), 1.12 (d, 6H); LCMS (m/z): 547.2 [M+H]⁺.

### EXAMPLE 93

### Preparation of N-{(1S)-1-[({3-[({4-bromo-2-[(trifluoromethyl)oxy]phenyl}sulfonyl)(methyl)amino]propyl}amino)carbonyl]-3-methylbutyl}-1-benzothiophene-2-carboxamide

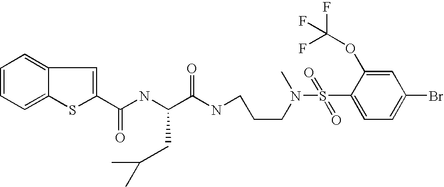

The title compound was prepared following the general procedure of Example 84 except substituting 2,4-dichlorobenzenesulfonyl chloride for 4-bromo-2-[(trifluoromethyl)oxy]benzenesulfonyl chloride: ¹H NMR (CDCl₃): δ 7.38-7.90 (m, 8H), 6.61-6.86 (m, 2H), 4.65-4.75 (m, 1H), 3.18-3.48 (m, 4H), 2.86 (s, 3H), 1.68-2.06 (m, 5H), 1.07 (d, 6H); LCMS (m/z): 664.0 [M+H]⁺.

### EXAMPLE 94

### Preparation of N-((1S)-1-{[((3R)-4-(Acetylamino)-3-{[(2-chloro-4-fluorophenyl)sulfonyl]amino}butyl)amino]carbonyl}-3-methylbutyl)-1-benzothiophene-2-carboxamide

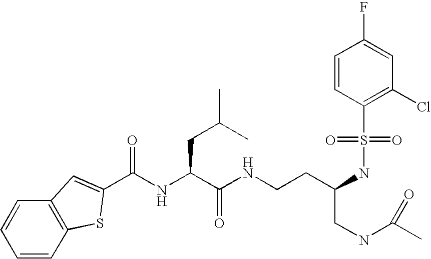 a) 1,1-Dimethylethyl [(3R)-4-hydroxy-3-({[(phenylmethyl)oxy]carbonyl}amino)butyl]carbamate

To a solution of 1,1-dimethylethyl (3-aminopropyl)carbamate (0.23 mL, 1.306 mmol) in CH₂Cl₂ (12 mL) were added N-(1-benzothien-2-ylcarbonyl)-L-leucine (418 mg, 1.436 mmol), HOOBt (11 mg, 0.065 mmol), and N-methylmorpholine (0.19 ml, 1.70 mmol) at RT. The mixture was stirred for several minutes whereupon EDC.HCl (288 mg, 1.502 mmol) was added. The reaction mixture was stirred overnight at RT. The reaction mixture was quenched with 10% (w/w) citric acid (25 mL), and extracted with dichloromethane (15 mL×2) followed by washing with saturated aqueous NaHCO₃ solution and brine. After drying over MgSO₄ and concentration, the residue was purified by flash column chromatography on silica gel (Biotage, 20% to 60% EtOAc/hexane) to provide 0.467 g of the title compound (80%): LCMS (m/z): 448.2 [MH]⁺.

### EXAMPLE 95

### Preparation of N-((1S)-1-{[(3-{[(2,4-Dichlorophenyl)sulfonyl]amino}propyl)amino]carbonyl}-3-methylbutyl)-1-benzothiophene-2-carboxamide

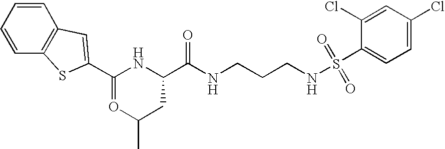 a. N-((1S)-1-{[(3-Aminopropyl)amino]carbonyl}-3-methylbutyl)-1-benzothiophene-2-carboxamide (TFA salt)

To a solution of 1,1-dimethylethyl (3-{[N-(1-benzothien-2-ylcarbonyl)-L-leucyl]amino}propyl)carbamate (67 mg, 0.15 mmol) in CH₂Cl₂ (1 mL) was added trifluoroacetic acid (1 mL) at RT. After stirring for 1 hr at RT, the reaction mixture was concentrated and dried under the reduced pressure. The resultant residue was carried over the next reaction without further purification.

b. N-((1S)-1-{[(3-{[(2,4-Dichlorophenyl)sulfonyl]amino}propyl)amino]carbonyl}-3-methylbutyl)-1-benzothiophene-2-carboxamide

To a solution of N-((1S)-1-{[(3-aminopropyl)amino]carbonyl}-3-methylbutyl)-1-benzothiophene-2-carboxamide (TFA salt) (0.15 mmol, crude) in CH₂Cl₂ were added 2,4-dichlorobenzenesulfonyl chloride (42 mg, 0.17 mmol) and Et₃N (0.08 mL, 0.6 mmol) at RT. After stirring for 1 hr, the reaction mixture was concentrated and subjected to flash column chromatography on silica gel (Biotage, 10% to 95% EtOAc/hexane) to give 80 mg (96% for two steps) of the title compound: LCMS (m/z): 556.2 [MH]⁺.

### EXAMPLE 96

### Preparation of N-((1S)-1-{[(3-{[(2-Chloro-4-fluorophenyl)sulfonyl]amino}propyl)amino]carbonyl}-3-methylbutyl)-1-benzothiophene-2-carboxamide

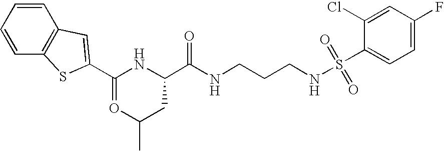

The title compound was prepared following the general procedure of Example 95 except substituting 2-chloro-4-fluorobenzenesulfonyl chloride: for 2,4-dichlorobenzenesulfonyl chloride: LCMS (m/z): 540.2 [MH]⁺.

### EXAMPLE 97

### Preparation of N-((1S)-1-{[(3-{[(2-Chloro-4-fluorophenyl)carbonyl]amino}propyl)amino]carbonyl}-3-methylbutyl)-1-benzothiophene-2-carboxamide

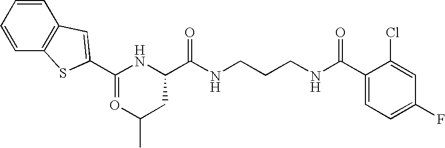

The title compound was prepared following the general procedure of Example 95 except substituting 2-chloro-4-fluorobenzoyl chloride: for 2,4-dichlorobenzenesulfonyl chloride: LCMS (m/z): 504.0 [MH]⁺.

### EXAMPLE 98

### Preparation of N-((1S)-1-{[{3-[[(2-Cyanophenyl)sulfonyl](methyl)amino]propyl}(methyl)amino]carbonyl}-3-methylbutyl)-1-methyl-1H-indole-2-carboxamide

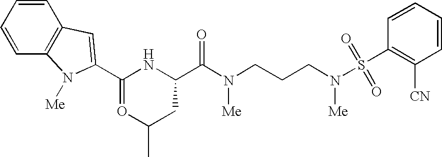 a. 2-Cyano-N-methyl-N-[3-(methylamino)propyl]benzenesulfonamide

To a solution of N,N′-dimethyl-1,3-propanediamine (1.24 mL, 9.92 mmol) in 50 mL of CH₂Cl₂ were added 2-cyanobenzenesulfonyl chloride (1.00 g, 4.96 mmol) and Et₃N (1.04 mL, 7.44 mmol) at 0° C. After stirring for 40 min at, the reaction mixture was warmed up to RT. After 30 min, the reaction mixture was extracted with 1N aqueous HCl solution (25 mL×2). The aqueous solution was basified (pH=12) with 6N aq. NaOH solution followed by extraction with CH₂Cl₂ (30 mL×2). The organic solution was dried over K₂CO₃. After filtration, concentration, and drying under a vacuum pump, the resultant residue was carried over to the next reaction without further purification.

b. N-((1S)-1-{[{3-[[(2-Cyanophenyl)sulfonyl](methyl)amino]propyl}(methyl)amino]carbonyl}-3-methylbutyl)-1-methyl-1H-indole-2-carboxamide

To a solution of crude 2-cyano-N-methyl-N-[3-(methylamino)propyl]benzenesulfonamide (120 mg, 0.45 mmol) in CH₂Cl₂ (4 mL) were added N-[(1-methyl-1H-indol-2-yl)carbonyl]-L-leucine (129 mg, 0.45 mmol), HOOBt (4 mg, 0.023 mmol), and N-methylmorpholine (0.07 ml, 0.675 mmol) at RT. The mixture was stirred for several minutes whereupon EDC.HCl (95 mg, 0.50 mmol) was added. The reaction mixture was stirred overnight at RT. The reaction mixture was quenched with 10% (w/w) citric acid (10 mL), and extracted with dichloromethane (10 mL×2) followed by washing with saturated aqueous NaHCO₃ solution and brine. After drying over MgSO₄ and concentration, the residue was purified by flash column chromatography on silica gel (Biotage, 20% to 60% EtOAc/hexane) to provide 0.193 g of the title compound (80%): LCMS (m/z): 538.2 [MH]⁺.

### EXAMPLE 99

### Preparation of N-((1S)-1-{[((2R,3S)-3-{[(2-chloro-4-fluorophenyl)sulfonyl]amino}-2,4-dihydroxybutyl)amino]carbonyl}-3-methylbutyl)-1-benzothiophene-2-carboxamide

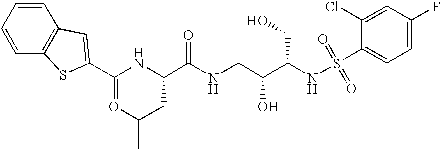 a. 2-Chloro-4-fluoro-N-[(1R)-1-(hydroxymethyl)-2-propen-1-yl]benzenesulfonamide

To a solution of (2R)-2-amino-3-buten-1-ol (benzoic acid salt, 4.12 g, 19.69 mmol) in CH₂Cl₂ (100 mL) was added Et₃N (9.61 mL, 68.92 mmol) and 2-chloro-4-fluorobenzenesulfonyl chloride (4.51 g, 19.69 mmol). The reaction mixture was stirred at −15° C. for 3 hr. After concentration, the residue was purified by flash column chromatography on silica gel (Biotage, 10 to 70% EtOAC/hexane) to provide 4.3 g of the desired compound (74%).

b. 2-Chloro-4-fluoro-N-{(1S)-2-hydroxy-1-[(2S)-2-oxiranyl]ethyl}benzenesulfonamide

To a solution of 2-chloro-4-fluoro-N-[(1R)-1-(hydroxymethyl)-2-propen-1-yl]benzenesulfonamide (351 mg, 1.26 mmol) in CH₃CN (7 mL) and H₂O (3.5 mL) at 0° C. was added CF₃COCH₃ (1.26 mL, 10.06 mmol) and NaHCO₃ (423 mg, 5.04 mmol) followed by slow addition of oxone (1.55 g, 2.52 mmol) during 5 minute. After vigorous stirring for 2 hr at 0° C., 60 ml of H₂O was added and extracted with CHCl₃ (15 mL×4). The organic solution was washed with 0.1% Na₂S₂O₃ solution, saturated aq. NaHCO₃, and brine. After drying over MgSO₄, filtration, and concentration, the residue was subjected to flash column chromatography over silica gel (Biotage 25M, 1% MeOH/30% EtOAC/69% hexane) to provide 2-chloro-4-fluoro-N-{(1S)-2-hydroxy-1-[(2S)-2-oxiranyl]ethyl}benzenesulfonamide (218 mg, 41%) and 2-chloro-4-fluoro-N-{(1S)-2-hydroxy-1-[(2R)-2-oxiranyl]ethyl}benzenesulfonamide (183 mg, 34%).

c. N-[(1S,2R)-3-Azido-2-hydroxy-1-(hydroxymethyl)propyl]-2-chloro-4-fluorobenzenesulfonamide

To a solution of 2-chloro-4-fluoro-N-{(1S)-2-hydroxy-1-[(2S)-2-oxiranyl]ethyl}benzenesulfonamide (215 mg, 0.729 mmol) in MeOH (5 mL) and H₂O (0.5 mL) were added NaN₃ (142 mg, 2.19 mmol) and NH₄Cl (117 mg, 2.19 mmol). The reaction mixture was heated to reflux for 4 hr. After evaporation, the silica gel was added to the above residue and subjected to flash column chromatography on silica gel (Biotage, CH₂Cl₂ only to 5% MeOH/CH₂Cl₂) to give 221 mg of the desired product (89%).

d. N-((1S)-1-{[((2R,3S)-3-{[(2-Chloro-4-fluorophenyl)sulfonyl]amino}-2,4-dihydroxybutyl)amino]carbonyl}-3-methylbutyl)-1-benzothiophene-2-carboxamide

To a solution of N-[(1S,2R)-3-azido-2-hydroxy-1-(hydroxymethyl)propyl]-2-chloro-4-fluorobenzenesulfonamide (221 mg, 0.65 mmol) in MeOH (3.5 mL) was added 10% Pd/C (103 mg, 0.098 mmol). The reaction mixture was stirred for 4 hr at rt under H2 (a balloon pressure). After filtration, the reaction mixture was concentrated and dried under the reduced pressure. To a solution of the above residue in CH₂Cl₂ (7 mL) were added N-(1-benzothien-2-ylcarbonyl)-L-leucine (199 mg, 0.68 mmol), HOOBt (5.3 mg, 0.03 mmol), and N-methylmorpholine (0.09 ml, 0.81 mmol). The mixture was stirred several minutes whereupon EDC HCl (143 mg, 0.75 mmol) was added. The reaction mixture was stirred at RT overnight. The reaction mixture was washed with cold 1N HCl solution, saturated aqueous NaHCO₃, and brine. The organic layer was dried over MgSO₄, filtered, and concentrated. Purification of the residue by flash column chromatography on silica gel (Biotage, 0.05:3:3:4=MeOH:CH₂Cl₂:EtOAC:hexane) provided 158 mg (42% for two steps) of the title compound: LCMS (m/z): 586.2 [M+H]⁺.

### EXAMPLE 100

### Preparation of N-[(1S)-2-[(4-{[(2,4-Dichlorophenyl)sulfonyl]amino}butyl)amino]-2-oxo-1-(1,3-thiazol-4-ylmethyl)ethyl]-1-benzothiophene-2-carboxamide

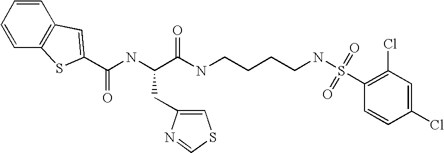

The title compound was prepared following the procedure of Example (need to refer to another example) except for the substitution of 1,4-butanediamine for 1,3-propanediamine and substituting 3-(1,3-thiazol-4-yl)-L-alanine for Boc-L-Beta-homoleucine: LCMS (m/z): 611.0 [MH]⁺

### EXAMPLE 101

### Preparation of N-((1S)-1-{[(3-{[(2,4-Dichlorophenyl)sulfonyl]amino}propyl)amino]carbonyl}-3-hydroxypropyl)-1-benzothiophene-2-carboxamide

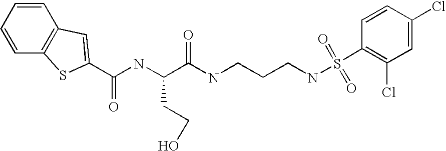

The title compound was prepared following the procedure of Example except for the substitution of L-homoserine for Boc-L-Beta-homoleucine: LCMS (m/z): 544.5 [MH]⁺

### EXAMPLE 102

### Preparation of N-((1S)-3,3-dichloro-1-{[(3-{[(2,4-dichlorophenyl)sulfonyl]amino}propyl)amino]carbonyl}propyl)-1-benzothiophene-2-carboxamide

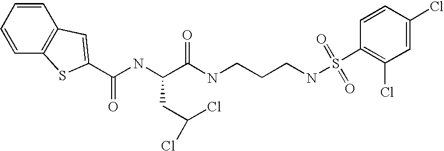

The title compound was prepared following the procedure of Example 98 except for the substitution of 1,3-propanediamine for N,N′-dimethyl-1,3-propanediamine and the substitution of 2,4-dichlorobenzene sulfonyl chloride for 2-cyanobnezene sulfonyl chloride in step 98a. In step 98b (2S)-2-[(1-benzothien-2-ylcarbonyl)amino]-4,4-dichlorobutanoic acid was substituted for N-[(1-methyl-1H-indol-2-yl)carbonyl]-L-leucine. The (2S)-2-[(1-benzothien-2-ylcarbonyl)amino]-4,4-dichlorobutanoic acid was synthesized using (2S)-2-amino-4,4-dichlorobutanoic acid [D. Winkler, K. Burger, Synthesis, 1419 (1996)]: LCMS (m/z): 598.0 [MH]⁺

### EXAMPLE 103

### Preparation of N-{(1S)-1-[({(2R)-3-[[(2-cyanophenyl)sulfonyl](methyl)amino]-2-hydroxypropyl}amino)carbonyl]-3-methylbutyl}-1-benzothiophene-2-carboxamide

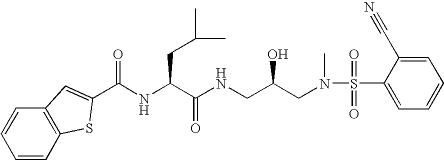 a. N-methyl-2-nitrobenzenesulfonamide

To a solution of triethylamine (1.4 ml, 10.14 mmol) in CH₂Cl₂ (30 mL) was added 2 M MeNH₂ in THF solution (4.06 ml, 8.12 mmol) at 0° C. followed by the addition of 2-nitrobenzenesulfonyl chloride (1.5 ml, 6.76 mmol). After stirring for 10 min at 0° C., the reaction mixture was warmed up to room temperature and kept stirring for 30 min. The mixture was quenched with 1N aqueous HCl and extracted with CH₂Cl₂ (30 mL×2). The organic solution was washed with saturated aqueous NaHCO₃ and brine, dried over MgSO₄, filtered, and concentrated. The crude material (1.4 g) was used in the next step without further purification: ¹H NMR (CDCl₃): δ 8.18 (d, 1H), 7.89 (d, 1H), 7.71-7.84 (m, 2H), 5.25 (brs, 1H), 2.80 (s, 3H); LCMS (m/z): 217.2 [M+H]⁺.

b. N-methyl-2-nitro-N-[(2S)-2-oxiranylmethyl]benzenesulfonamide

To a solution of N-methyl-2-nitrobenzenesulfonamide (2.38 g, 11.05 mmol) in THF (50 mL) was added PPh₃ (4.35 g, 16.57 mmol), DEAD (2.61 ml, 16.57 mmol), and (2S)-2-oxiranylmethanol (1.1 ml, 16.57 mmol). This mixture was stirred at room temperature overnight. THF was removed and the residue was purified by flash column chromatography on silica gel (Biotage, 10%-20% EtOAc/hexane, then 500 ml of CH₂Cl₂) to provide the title compound (2.81 g, 93.7%).

c. N-[(2R)-3-azido-2-hydroxypropyl]-N-methyl-2-nitrobenzenesulfonamide

To the solution of N-methyl-2-nitro-N-[(2S)-2-oxiranylmethyl]benzenesulfonamide (2.81 g, 10.33 mmol) in MeOH/H₂O (10:1) were added NaN₃ (1.7 g, 31.99 mmol) and NH₄Cl (2.42 g, 37.31 mmol). The reaction mixture was refluxed for 3 hr. Removed most of the solvent and added H₂O (300 ml), and extracted with dichloromethane (100 mL×3). The organic solution was dried over MgSO₄, filtered, and concentrated. After drying under the reduced pressure, the residue was carried out to the next step without further purification.

d. N-[(2R)-3-amino-2-hydroxypropyl]-N-methyl-2-nitrobenzenesulfonamide

To a solution of crude N-[(2R)-3-azido-2-hydroxypropyl]-N-methyl-2-nitrobenzenesulfonamide in THF/H₂O (10:1) was added PPh₃. The reaction mixture was refluxed overnight. After solvents were evaporated, cold 1N HCl (60 ml) and ethyl acetate (100 ml) were added. The organic layer was extracted with 1N HCl (50 ml). The combined aqueous layer was first washed with ethyl acetate. The aqueous solution was adjusted to pH 12-12.5 with 1N NaOH and the mixture was extracted with dichloromethane (30 mL×5). The organic layer was washed with brine, dried over K₂CO₃, and concentrated. The residue was carried out to the next step without further purification.

e. N-((1S)-1-{[((2R)-2-hydroxy-3-{methyl[(2-nitrophenyl)sulfonyl]amino}propyl)amino]carbonyl}-3-methylbutyl)-1-benzothiophene-2-carboxamide

To a solution of N-[(2R)-3-amino-2-hydroxypropyl]-N-methyl-2-nitrobenzenesulfonamide (980 mg, 3.37 mmol) in CH₂Cl₂ were added N-(1-benzothien-2-ylcarbonyl)-L-leucine (1.03 g, 3.54 mmol), HOOBt (27 mg, 0.168 mmol), and N-methylmorpholine (0.48 ml, 4.38 mmol). The mixture was stirred several minutes whereupon EDC HCl (710 mg, 3.71 mmol) was added. The reaction mixture was stirred at RT overnight. The reaction mixture was washed with cold 1N HCl solution, saturated aqueous NaHCO₃, and brine. The organic layer was dried over MgSO₄, filtered, and concentrated. Purification of the residue by flash column chromatography on silica gel (Biotage, 0%-1% MeOH/CH₂Cl₂) provided 1.3 g of the title compound (69%): ¹H NMR (CDCl₃): δ 7.38-8.08 (m, 9H), 6.71-7.05 (m, 2H), 4.71-4.79 (m, 1H), 3.28-4.01 (m, 4H), 3.08-3.15 (m, 1H), 2.98 (s, 3H), 1.70-1.91 (m, 3H), 1.01 (d, 6H); LCMS (m/z): 563.6 [M+H]⁺.

f. N-[(1S)-1-({[(2S)-2-hydroxy-3-(methylamino)propyl]amino}carbonyl)-3-methylbutyl]-1-benzothiophene-2-carboxamide

To a solution of N-((1S)-1-{[((2R)-2-hydroxy-3-{methyl[(2-nitrophenyl)sulfonyl]amino}propyl)amino]carbonyl}-3-methylbutyl)-1-benzothiophene-2-carboxamide (384 mg, 0.68 mmol) in DMF (3 mL) was added benzenethiol (0.14 ml, 1.37 mmol) and K₂CO₃ (425 mg, 3.07 mmol). The reaction mixture was stirred at RT for 5 hr. After the solid was filtered off, ethyl acetate (50 ml) and water (50 mL) were added. After extraction with 1N HCl/ice water (1:1) three times (30 ml, 20 ml, and 10 ml), the combined aqueous solution was washed with CH₂Cl₂ twice. The aqueous solution was basified to pH 13 with 6N NaOH followed by extraction with CH₂Cl₂ three times and washing with saturated NaHCO₃ and brine. The organic layer was dried over K₂CO₃, filtered, and concentrated to provide 170 mg of the title compound, which was used for the next step without further purification.

g. N-{(1S)-1-[({(2R)-3-[[(2-cyanophenyl)sulfonyl](methyl)amino]-2-hydroxypropyl}amino)carbonyl]-3-methylbutyl}-1-benzothiophene-2-carboxamide

To a suspension of N-[(1S)-1-({[(2S)-2-hydroxy-3-(methylamino)propyl]amino}carbonyl)-3-methylbutyl]-1-benzothiophene-2-carboxamide (64 mg, 0.17 mmol) in CH₂Cl₂ (3 mL) and saturated aqueous NaHCO₃ (1.5 ml). Cooled down the mixture to 0° C., and then added 2-cyanobenzenesulfonyl chloride (44 mg, 0.22 mmol). The reaction mixture was stirred at 0° C. for 5 min, then warmed up to RT, kept stirring for additional 35 min. The residue was diluted with water (15 ml) and extracted twice with dichloromethane. The combined organic solution was washed with brine and dried over MgSO₄, filtered, and concentrated. The residue was purified by flash column chromatography on silica gel (Biotage, 0% to 1.5% MeOH/CH₂Cl₂) to provide 81 mg of the final compound (88%): ¹H NMR (CDCl₃): δ 7.42-8.11 (m, 9H), 7.02-7.21 (m, 2H), 4.70-4.82 (m, 1H), 3.85-4.02 (m, 2H), 3.35-3.61 (m, 2H), 2.98 (s, 3H), 2.75-2.83 (m, 1H), 1.74-1.99 (m, 3H), 1.03 (d, 6H) 2.85 (s, 3H); LCMS (m/z): 543.2 [M+H]⁺.

### EXAMPLE 104

### Preparation of N-[(1S)-2-({(2R)-3-[[(2-cyanophenyl)sulfonyl](methyl)amino]-2-hydroxypropyl}amino)-1-(cyclohexylmethyl)-2-oxoethyl]-1-benzothiophene-2-carboxamide

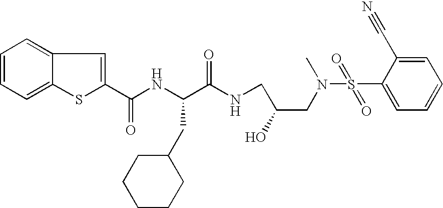

The title compound was prepared following the general procedure of Example 103 except using the product from step 103d to couple with Boc-β-Cha-OH to make 1,1-dimethylethyl acetate-3-cyclohexyl-N¹-((2R)-2-hydroxy-3-{methyl[(2-nitrophenyl)sulfonyl]amino}propyl)-L-alaninamide, then followed by deprotection of the Boc protecting group using 4N HCl in dioxane reagent to provide 3-cyclohexyl-N¹-((2R)-2-hydroxy-3-{methyl[(2-nitrophenyl)sulfonyl]amino}propyl)-L-alaninamide (HCl salt). This intermediate was then coupled with 1-benzothiophene-2-carboxylic acid by using EDC.HCl, HOBt, and TEA. This coupling product was then used in steps 103f and 103g to provide the final title compound: ¹H NMR (CDCl₃): δ 7.38-8.09 (m, 9H), 7.03-7.30 (m, 2H), 4.71-4.90 (m, 1H), 3.30-4.05 (m, 4H), 2.93 (s, 3H), 0.95-2.04 (m, 13H); LCMS (m/z): 583.4 [M+H]⁺.

### EXAMPLE 105

### Preparation of N-[(1S)-1-(cyclohexylmethyl)-2-({(2R)-3-[[(2,4-dichlorophenyl)sulfonyl](methyl)amino]-2-hydroxypropyl}amino)-2-oxoethyl]-1-benzothiophene-2-carboxamide

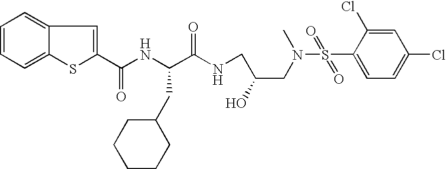

The title compound was prepared following the general procedure of Example 104 except substituting 2-cyanobenzenesulfonyl chloride for 2,4-dichlorobenzenesulfonyl chloride: ¹H NMR (CDCl₃): 7.36-8.07 (m, 8H), 6.62-6.98 (m, 2H), 4.65-4.71 (m, 1H), 3.32-4.06 (m, 4H), 3.15-3.22 (m, 1H), 2.93 (s, 3H), 0.95-2.02 (m, 13H); LCMS (m/z): 626.2 [M+H]⁺.

### EXAMPLE 106

### Preparation of N-[(1S)-2-({(2R)-3-[[(2-cyanophenyl)sulfonyl](methyl)amino]-2-hydroxypropyl}amino)-1-(cyclopentylmethyl)-2-oxoethyl]-1-benzothiophene-2-carboxamide

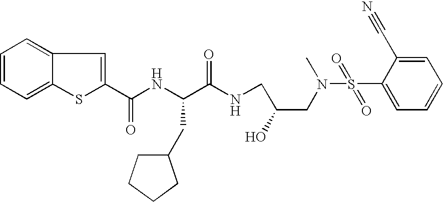

The title compound was prepared following the general procedure of Example 103 except substituting N-(1-benzothien-2-ylcarbonyl)-3-cyclopentyl-L-alanine for N-(1-benzothien-2-ylcarbonyl)-L-leucine: ¹H NMR (CDCl₃): δ 7.35-8.08 (m, 9H), 7.03-7.18 (m, 2H), 4.72-4.78 (m, 1H), 4.42-4.45 (m, 1H), 3.89-4.03 (m, 2H), 3.38-3.61 (m, 2H), 2.98 (s, 3H), 2.81-2.89 (m, 1H), 1.73-2.16 (m, 5H), 1.50-1.55 (m, 2H), 1.16-1.25 (m, 2H); LCMS (m/z): 569.2 [M+H]⁺.

### EXAMPLE 107

### Preparation of N-[(1S)-1-(cyclopentylmethyl)-2-({(2R)-3-[[(2,4-dichlorophenyl)sulfonyl](methyl)amino]-2-hydroxypropyl}amino)-2-oxoethyl]-1-benzothiophene-2-carboxamide

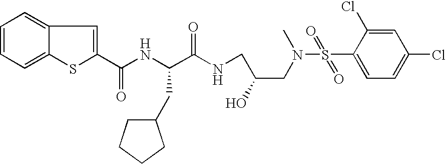

The title compound was prepared following the general procedure of Example 106 except for substituting 2,4-dichlorobenzenesulfonyl chloride for 2-cyanobenzenesulfonyl chloride: ¹H NMR (CDCl₃): δ 7.29-8.03 (m, 8H), 6.82-7.08 (m, 2H), 4.62-4.68 (m, 1H), 3.95-4.08 (m, 2H), 3.70-3.80 (m, 1H), 3.18-3.44 (m, 3H), 2.91 (s, 3H), 1.80-2.07 (m, 6H), 1.45-1.58 (m, 2H), 1.16-1.22 (m, 2H); LCMS (m/z): 612.3 [M+H]⁺.

### EXAMPLE 108

### Preparation of N-{(1S)-1-[({(2R)-3-[[(2-chloro-4-fluorophenyl)sulfonyl](methyl)amino]-2-hydroxypropyl}amino)carbonyl]-3-methylbutyl}-1-benzothiophene-2-carboxamide

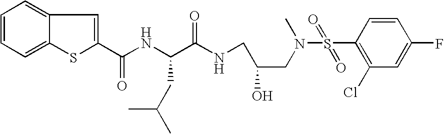

The title compound was prepared following the general procedure of Example 103 except substituting 2-chloro-4-fluorobenzenesulfonyl chloride for 2-cyanobenzenesulfonyl chloride: ¹H NMR (CDCl₃): δ 7.12-8.13 (m, 8H), 6.74-7.05 (m, 2H), 4.65-4.76 (m, 1H), 3.72-4.04 (m, 2H), 3.18-3.42 (m, 3H), 2.94 (s, 3H), 1.64-1.91 (m, 3H), 1.03 (d, 6H); LCMS (m/z): 570.2 [M+H]⁺.

### EXAMPLE 109

### Preparation of N-[(1S)-2-({(2R)-3-[[(2-chloro-4-fluorophenyl)sulfonyl](methyl)amino]-2-hydroxypropyl}amino)-1-(cyclohexylmethyl)-2-oxoethyl]-1-benzothiophene-2-carboxamide

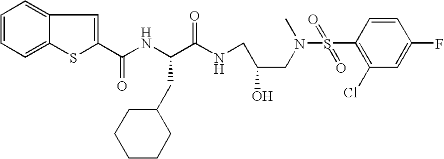

The title compound was prepared following the general procedure of Example 104 except substituting 2-chloro-4-fluorobenzenesulfonyl chloride for 2-cyanobenzenesulfonyl chloride: ¹H NMR (CDCl₃): δ 7.15-8.14 (m, 8H), 6.65-6.96 (m, 2H), 4.65-4.75 (m, 1H), 4.06 (m, 1H), 3.18-3.48 (m, 4H), 2.95 (s, 3H), 0.95-1.95 (m, 13H); LCMS (m/z): 610.2 [M+H]⁺.

### EXAMPLE 110

### Preparation of N-{(1S)-1-[({(2R)-3-[[(2,4-dichlorophenyl)sulfonyl](methyl)amino]-2-hydroxypropyl}amino)carbonyl]-3-methylbutyl}-1-benzothiophene-2-carboxamide

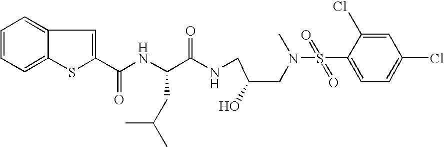

The title compound was prepared following the general procedure of Example 103 except substituting 2,4-dichlorobenzenesulfonyl chloride for 2-cyanobenzenesulfonyl chloride: ¹H NMR (CDCl₃): δ 7.38-8.04 (m, 8H), 6.65-7.01 (m, 2H), 4.65-4.76 (m, 1H), 3.72-4.04 (m, 2H), 3.18-3.44 (m, 3H), 2.94 (s, 3H), 1.64-1.90 (m, 3H), 1.03 (d, 6H); LCMS (m/z): 586.2 [M+H]⁺.

### EXAMPLE 111

### Preparation of N-{(1S)-1-[({(2S)-3-[[(2,4-dichlorophenyl)sulfonyl](methyl)amino]-2-hydroxypropyl}amino)carbonyl]-3-methylbutyl}-1-benzothiophene-2-carboxamide

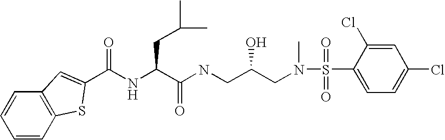

The title compound was prepared following the general procedure of Example 110 except substituting (2S)-2-oxiranylmethanol for (2R)-2-oxiranylmethanol: LCMS (m/z): 586.0 [M+H]⁺.

### EXAMPLE 112

### Preparation of N-((1S)-1-{[((2S)-2-hydroxy-3-{methyl[(2-nitrophenyl)sulfonyl]amino}propyl)amino]carbonyl}-3-methylbutyl)-1-benzothiophene-2-carboxamide

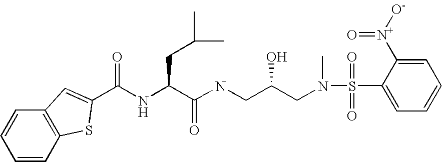

The title compound was prepared following the general procedure of Example 103e except substituting (2S)-2-oxiranylmethanol for (2R)-2-oxiranylmethanol: LCMS (m/z): 563.2 [M+H]⁺.

### EXAMPLE 113

### Preparation of N-((1S)-1-{[((2R)-2-hydroxy-3-{methyl[(2-nitrophenyl)sulfonyl]amino}propyl)amino]carbonyl}-3-methylbutyl)-1-benzothiophene-2-carboxamide

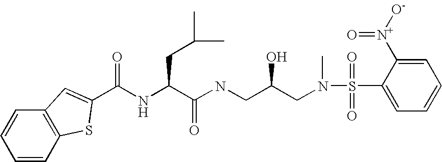

The preparation of the title compound was described in the general procedure of Example 103e: LCMS (m/z): 563.2 [M+H]⁺.

### EXAMPLE 114

### Preparation of N-{(1S)-1-[({(2S)-3-[[(2-cyanophenyl)sulfonyl](methyl)amino]-2-hydroxypropyl}amino)carbonyl]-3-methylbutyl}-1-benzothiophene-2-carboxamide

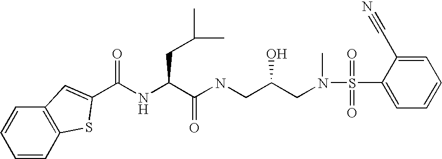

The title compound was prepared following the general procedure of Example 103 except substituting (2S)-2-oxiranylmethanol for (2R)-2-oxiranylmethanol: LCMS (m/z): 543.2 [M+H]⁺.

### EXAMPLE 115

### N-{(1S)-1-[({3-[[(2,4-dichlorophenyl)sulfonyl](methyl)amino]-2-oxopropyl}amino)carbonyl]-3-methylbutyl}-1-benzothiophene-2-carboxamide

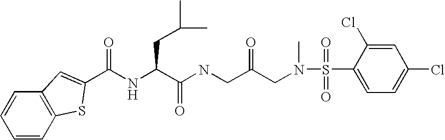

To a solution of N-{(1S)-1-[({(2S)-3-[[(2,4-dichlorophenyl)sulfonyl](methyl)amino]-2-hydroxypropyl}amino)carbonyl]-3-methylbutyl}-1-benzothiophene-2-carboxamide (Example 111, 148 mg, 0.25 mmol) in CH₂Cl₂ (5 mL) was added Dess-Martin periodinane (161 mg, 0.38 mmol) at rt. After stirring for 2 hr, the reaction mixture was diluted with 15 mL of CH₂Cl₂ and washed with 10% aq. Na₂S₂O₃ solution, saturated aq. NaHCO₃, and brine. The organic solution was dried over MgSO₄, filtered, and concentrated. The residue was purified by flash column chromatography on silica gel (Biotage, 10% to 60% EtOAc/hexane) to provide 127 mg of the final compound (70%): LCMS (m/z): 584.0 [M+H]⁺.

### EXAMPLE 116

### Preparation of N-[(1S)-1-({[3-[[(2,4-dichlorophenyl)sulfonyl](methyl)amino]-2-(methylamino)propyl]amino}carbonyl)-3-methylbutyl]-1-benzothiophene-2-carboxamide

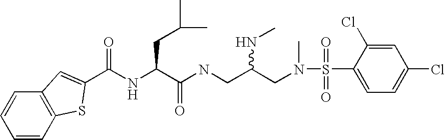

To a solution of N-{(1S)-1-[({3-[[(2,4-dichlorophenyl)sulfonyl](methyl)amino]-2-oxopropyl}amino)carbonyl]-3-methylbutyl}-1-benzothiophene-2-carboxamide (Example 115, 64 mg, 0.11 mmol) in absolute MeOH (3 mL) were added MeNH₂HCl (200 mg, 2.96 mmol), NaCNBH₃ (21 mg, 0.33 mmol), and 4M HCl (0.03 mL, 0.11 mmol) in dioxane at rt. After stirring for 2.5 day, the reaction mixture was concentrated and the residue was dissolved with ethyl acetate (15 ml) and 1N HCl (10 mL) were added. After extraction with 1N HCl/ice water (1:1) (5 ml×2), the combined aqueous solution was washed with ethyl acetate twice. The aqueous solution was basified to pH 13 with 1N NaOH followed by extraction with CH₂Cl₂ three times and washing with saturated NaHCO₃ and brine. The organic solution was dried over K₂CO₃, filtered, and concentrated. The residue was purified by flash column chromatography on silica gel (Biotage, 0% to 5% MeOH/CH₂Cl₂) to provide 23 mg of the title compound (35%): LCMS (m/z): 599.4 [M+H]⁺.

### EXAMPLE 117

### Preparation of N-{(1S)-1-(cyclohexylmethyl)-2-[((2R)-2-hydroxy-3-{methyl[(2-nitrophenyl)sulfonyl]amino}propyl)amino]-2-oxoethyl}-1-benzothiophene-2-carboxamide

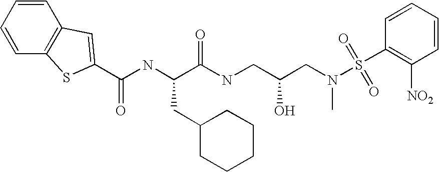

The title compound was prepared according to the general procedure of Example 104 up to step 104e: LCMS (m/z): 603 [M+H]⁺.

### EXAMPLE 118

### Preparation of N-[(1S)-2-({(2R)-3-[[(2-cyanophenyl)sulfonyl](methyl)amino]-2-hydroxypropyl}amino)-1-(cyclohexylmethyl)-2-oxoethyl]-1-methyl-1H-indole-2-carboxamide

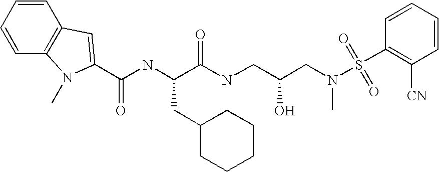

The title compound was prepared according to the procedure of Example 104 except substituting 1-methyl-1H-indole-2-carboxylic acid for 1-benzothiophene-2-carboxylic acid: LCMS (m/z): 580.4 [M+H]⁺.

### EXAMPLE 119

### N-{(1S)-1-(cyclopentylmethyl)-2-[((2R)-2-hydroxy-3-{methyl[(2-nitrophenyl)sulfonyl]amino}propyl)amino]-2-oxoethyl}-1-benzothiophene-2-carboxamide

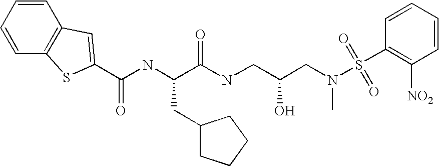

The title compound was prepared following the procedure of Example 103 except substituting N-(1-benzothien-2-ylcarbonyl)-3-cyclopentyl-L-alanine for N-(1-benzothien-2-ylcarbonyl)-L-leucine and omission of step 103f and 103 g: LCMS (m/z): 589.2 [M+H]⁺.

### EXAMPLE 120

### Preparation of N-[(1S)-1-({[3-[[(2,4-Dichlorophenyl)sulfonyl](methyl)amino]-2-(methyloxy)propyl]amino}carbonyl)-3-methylbutyl]-1-benzothiophene-2-carboxamide

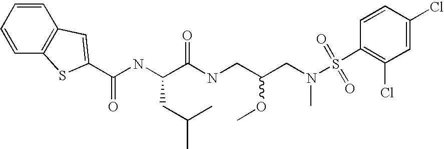 a) N-(3-Amino-2-hydroxypropyl)-2,4-dichlorobenzenesulfonamide

To a solution of 1,3-diamino-2-propanol (4.42 g, 48.9 mmol) in dichloromethane (250 mL) at 0° C. was added triethylamine (6.81 mL, 48.9 mmol) and 2,4-dichlorobenzenesulfonyl chloride. After stirring the mixture for 2.5 h, it was quenched and acidified to pH=2 using aq. 1 N HCl. After the organic layer was separated, the aqueous layer was basified to pH=11, then extracted with ethylacetate (150 mL×2). The latter organic layer was dried over MgSO₄, filtered, then concentrated to obtain the title compound as a white solid that was used in the next step without further purification (3.71 g, 76%): LCMS (m/z): 299.0 [M+H]⁺.

b) 1,1-Dimethylethyl (3-{[(2,4-dichlorophenyl)sulfonyl]amino}-2-hydroxypropyl)carbamate

To a solution of N-(3-amino-2-hydroxypropyl)-2,4-dichlorobenzenesulfonamide (1.50 g, 5.03 mmol) in THF (20 mL) at 0° C. was added di-tert-butyl-dicarbonate (1.15 g, 5.28 mmol). After stirring overnight at RT, the mixture was concentrated under reduced pressure and a residue was purified by silica gel flash chromatography (Biotage, 0% to 3.0%, MeOH/DCM) to obtain the title compound (2.0 g, 100%): LCMS (m/z): 299.0 [(M−100)+H]⁺.

c) 1,1-Dimethylethyl {3-[[(2,4-dichlorophenyl)sulfonyl](methyl)amino]-2-hydroxypropyl}carbamate

To a solution of 1,1-dimethylethyl (3-{[(2,4-dichlorophenyl)sulfonyl]amino}-2-hydroxypropyl)carbamate (1.30 g, 3.27 mmol) in acetone was added K₂CO₃ (2.26 g, 16.3 mmol) and CH₃I (0.612 mL, 9.80 mmol). The reaction mixture was vigorously stirred for 3 hr, then diluted with dichloromethane, filtered, and concentrated under reduced pressure. The residue was purified by silica gel flash chromatography (Biotage, 0% to 2.5%, MeOH/dichloromethane) to obtain the title compound (1.29 g, 96%): LCMS (m/z): 313.0 [(M−100)+H]⁺.

d) 1,1-Dimethylethyl[3-[[(2,4-dichlorophenyl)sulfonyl](methyl)amino]-2-(methyloxy)propyl]carbamate

To a solution of 1,1-dimethylethyl {3-[[(2,4-dichlorophenyl)sulfonyl](methyl)amino]-2-hydroxypropyl}carbamate (300 mg, 0.728 mmol) in THF at 0° C. was added NaH (60%, 29.1 mg, 0.728 mmol). The mixture was stirred for 5 min at 0° C. then 60 min at RT, then cooled to 0° C. whereupon methyl iodide (0.045 mL, 0.728 mmol) was added. After stirring for 2 h at RT, it was quenched with water (10 mL), extracted with dichloromethane (50 mL×2), dried over MgSO₄ and concentrated under the reduced pressure. The residue was purified by silica gel flash chromatography (Biotage, 0% to 20%, THF/hexane) to provide the title compound (203 mg, 65%): LCMS (m/z): 327.0 [(M−100)+H]⁺.

e) N-[(1S)-1-({[3-[[(2,4-Dichlorophenyl)sulfonyl](methyl)amino]-2-(methyloxy)propyl]amino}carbonyl)-3-methylbutyl]-1-benzothiophene-2-carboxamide

To a solution of 1,1-dimethylethyl [3-[[(2,4-dichlorophenyl)sulfonyl](methyl)amino]-2-(methyloxy)propyl]carbamate (190 mg, 0.446 mmol) in dichloromethane (4 mL) was added a solution of 4 N HCl in dioxane (1.34 mL, 5.35 mmol). After stirring for 3 h at RT, the solvent was concentrated under the reduced pressure and the residue azeotroped using toluene. To a solution of the crude residue in dichloromethane (3.5 mL) was added N-(1-benzothien-2-ylcarbonyl)-L-leucine (143 mg, 0.491 mmol), HOOBt (1.81 mg, 0.011 mmol), NMM (0.147 mL, 1.34 mmol), and EDC.HCl (94.0 mg, 0.491 mmol). After stirring overnight at RT, the mixture was washed with aq. 10% citric acid, saturated aq. NaHCO₃, and brine. After drying over MgSO₄ and concentration under reduced pressure, the residue was purified by silica gel chromatography (Biotage, 25% to 40%, EtOAc/hexane) to obtain the title compound: LCMS (m/z): 600.2 [M+H]⁺.

### EXAMPLE 121

### Preparation of N-[(1S)-1-({[3-[[(2,4-dichlorophenyl)sulfonyl](methyl)amino]-2-(2-propen-1-yloxy)propyl]amino}carbonyl)-3-methylbutyl]-1-benzothiophene-2-carboxamide

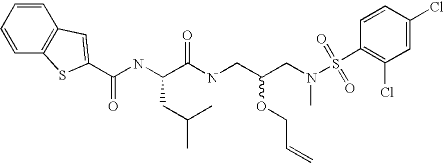

The title compound was synthesized following the procedure of Example 120 except substituting allyl bromide for methyl iodide in step 120b: LCMS (m/z): 626.2 [M+H]⁺.

### EXAMPLE 122

### Preparation of N-((1S)-1-{[((2R)-3-{[(2-chloro-4-fluorophenyl)sulfonyl]amino}-2-hydroxypropyl)amino]carbonyl}-3-methylbutyl)-1-benzothiophene-2-carboxamide

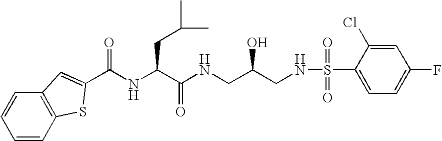 a. 2-[(2R)-2-oxiranylmethyl]-1H-isoindole-1,3(2H)-dione

To a solution of phthalimide (6.6 g, 45.0 mmol) in THF (220 mL) was added PPh₃ (17.7 g, 67.5 mmol), DEAD (11.7 ml, 67.5 mmol), and (R)-(+)-glycidol (4.0 g, 54.0 mmol). This mixture was stirred at room temperature overnight. THF was removed and the residue was purified by flash column chromatography on silica gel (Biotage, 5%-20% EtOAc/hexane) to provide the title compound (5.8 g, 75%).

b. 2-chloro-N-[(2R)-3-(1,3-dioxo-1,3-dihydro-2H-isoindol-2-yl)-2-hydroxypropyl]-4-fluorobenzenesulfonamide

To a solution of 2-[(2R)-2-oxiranylmethyl]-1H-isoindole-1,3(2H)-dione (2.0 g, 10.0 mmol) in isopropyl alcohol (40 mL) were added 2-chloro-4-fluorobenzenesulfonamide (2.51 g, 12.0 mmol) and pyridine (0.16 g, 2.0 mmol). The reaction mixture was refluxed for overnight. Solvent was removed and residue was purified by flash column chromatography on silica gel (Biotage, 0.5%-2% MeOH/CH₂Cl₂) to provide the title compound (2.64 g, 55.0%).

c. N-[(2R)-3-amino-2-hydroxypropyl]-2-chloro-4-fluorobenzenesulfonamide

To a solution of 2-chloro-N-[(2R)-3-(1,3-dioxo-1,3-dihydro-2H-isoindol-2-yl)-2-hydroxypropyl]-4-fluorobenzenesulfonamide (0.12 g) in EtOH (5 mL) was added hydrazine (0.05 mL). After stirring for overnight at room temperature, the reaction mixture was filtered and the filtrate was concentrated. The resultant residue was carried over the next step without further purification.

d. N-((1S)-1-{[((2R)-3-{[(2-chloro-4-fluorophenyl)sulfonyl]amino}-2-hydroxypropyl)amino]carbonyl}-3-methylbutyl)-1-benzothiophene-2-carboxamide

To a solution of N-[(2R)-3-amino-2-hydroxypropyl]-2-chloro-4-fluorobenzenesulfonamide (3.64 mmol) in CH₂Cl₂ (20 mL) were added N-(1-benzothien-2-ylcarbonyl)-L-leucine (1.21 g, 4.19 mmol), HOOBT (32.0 mg, 0.20 mmol), and N-methylmorpholine (1.20 ml, 10.9 mmol). The mixture was stirred several minutes whereupon EDC.HCl (0.83 g, 4.37 mmol) was added. The reaction mixture was stirred at RT overnight. The reaction mixture was washed with cold 1N HCl solution, saturated aqueous NaHCO₃, and brine. The organic layer was dried over MgSO₄, filtered, and concentrated. Purification of the residue by flash column chromatography on silica gel (Biotage, 0%-3% MeOH/CH₂Cl₂) provided 1.40 g of the title compound (69%): LCMS (m/z): 556.2 [M+H]⁺.

### EXAMPLE 123

### Preparation of N-((1S)-1-{[((2R)-3-{[(2,4-dichlorophenyl)sulfonyl]amino}-2-hydroxypropyl)amino]carbonyl}-3-methylbutyl)-1-benzothiophene-2-carboxamide

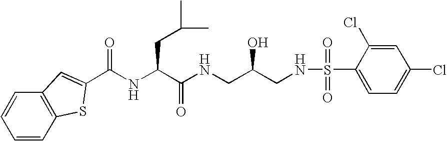

The title compound was prepared following the general procedure of Example 122 except substituting 2-chloro-4-fluorobenzenesulfonyl amide with 2,4-dichlorobenzenesulfonamide which was made by using 2,4-dichlorobenzenesulfonyl chloride reacting with ammonia: LCMS (m/z): 572.0 [MH]⁺.

### EXAMPLE 124

### Preparation of N-[(1S)-1-({[(2R)-3-({[4-fluoro-2-(trifluoromethyl)phenyl]sulfonyl}amino)-2-hydroxypropyl]amino}carbonyl)-3-methylbutyl]-1-benzothiophene-2-carboxamide

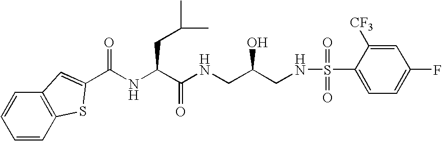

The title compound was prepared following the general procedure of Example 122 except substituting 2-chloro-4-fluorobenzenesulfonamide with 2-trifluoromethyl-4-chlorobenzenesulfonamide which is made by using 2-trifluoromethyl-4-chlorobenzenesulfonyl chloride to react with ammonia: LCMS (m/z): 590.2 [MH]⁺.

### EXAMPLE 125

### Preparation of N-[(1S)-2-[((2R)-3-{[(2-chloro-4-fluorophenyl)sulfonyl]amino}-2-hydroxypropyl)amino]-1-(cyclohexylmethyl)-2-oxoethyl]-1-benzothiophene-2-carboxamide

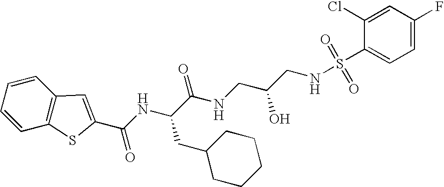

The title compound was prepared following the general procedure of Example 122 except using the product from step 1c to couple with Boc-β-Cha-OH to make 1,1-dimethylethyl [(1S)-2-[((2R)-3-{[(2-chloro-4-fluorophenyl)sulfonyl]amino}-2-hydroxypropyl)amino]-1-(cyclohexylmethyl)-2-oxoethyl]carbamate then followed by deprotection of Boc using 4N HCl in dioxane reagent to get N¹-((2R)-3-{[(2-chloro-4-fluorophenyl)sulfonyl]amino}-2-hydroxypropyl)-3-cyclohexyl-L-alaninamide (HCl salt), then coupling the intermediate with 1-benzothiophene-2-carboxylic acid by using EDC.HCl, HOBt, and TEA to obtain the title compound: LCMS (m/z): 596.2 [M+H]⁺.

### EXAMPLE 126

### Preparation of N²-[(cyclohexylamino)carbonyl]-N¹-((2R)-3-{[(2,4-dichlorophenyl)sulfonyl]amino}-2-hydroxypropyl)-L-leucinamide

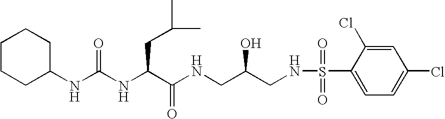

The title compound was prepared following the general procedure of Example 2 except using Boc-L-leucine to make N¹-((2R)-3-{[(2,4-dichlorophenyl)sulfonyl]amino}-2-hydroxypropyl)-N²-{[(1,1-dimethylethyl)oxy]carbonyl}-L-leucinamide then followed by deprotection of Boc using 4N HCl in dioxane reagent to N¹-((2R)-3-{[(2,4-dichlorophenyl)sulfonyl]amino}-2-hydroxypropyl)-L-leucinamide (HCl salt), then couple the intermediate with isocyanatocyclohexane and TEA to obtain the title compound: LCMS (m/z): 537.2 [M+H]⁺.

### EXAMPLE 127

### Preparation of N-((1S)-1-{[(3-{[(2,4-dichlorophenyl)sulfonyl]amino}-2-oxopropyl)amino]carbonyl}-3-methylbutyl)-1-benzothiophene-2-carboxamide

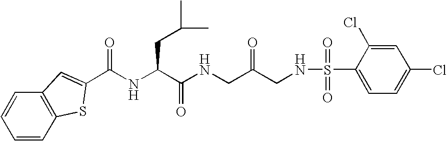

To a solution of N-((1S)-1-{[((2R)-3-{[(2,4-dichlorophenyl)sulfonyl]amino}-2-hydroxypropyl)amino]carbonyl}-3-methylbutyl)-1-benzothiophene-2-carboxamide (80 mg, 0.14 mmol) in dichloromethane (2.0 mL) was added Dess-Martin periodinane (89.0 mg, 0.21 mmol). After 2.5 h at RT, the reaction mixture was diluted with dichloromethane, washed with 10% aq. Na₂S₂O₃ solution, saturated aq. NaHCO₃, and brine. The organic layers were dried over MgSO₄ then concentrated under reduced pressure. The residue was purified by silica gel chromatography (Biotage, 25% to 60% EtOAc/hexane) to provide the title compound (46 mg, 58%): LCMS (m/z): 570.2 [M+H]⁺.

### EXAMPLE 128

### Preparation of N-((1S)-1-{[(3-{[(2-chloro-4-fluorophenyl)sulfonyl]amino}-2-oxopropyl)amino]carbonyl}-3-methylbutyl)-1-benzothiophene-2-carboxamide

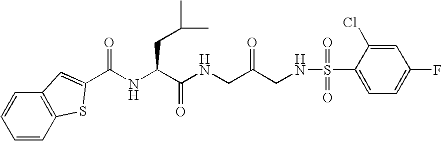

The title compound was obtained by following the procedure of example 6 except for the substitution of N-((1S)-1-{[((2R)-3-{[(2-chloro-4-fluorophenyl)sulfonyl]amino}-2-hydroxypropyl)amino]carbonyl}-3-methylbutyl)-1-benzothiophene-2-carboxamide for N-((1S)-1-{[((2R)-3-{[(2,4-dichlorophenyl)sulfonyl]amino}-2-hydroxypropyl)amino]carbonyl}-3-methylbutyl)-1-benzothiophene-2-carboxamide: LCMS (m/z): 554.2 [M+H]⁺.

### EXAMPLE 129

### Preparation of N-((1S)-1-{[(3-{[(4-Chlorophenyl)sulfonyl]amino}-1-ethylpropyl)amino]carbonyl}-3-methylbutyl)-1-benzothiophene-2-carboxamide

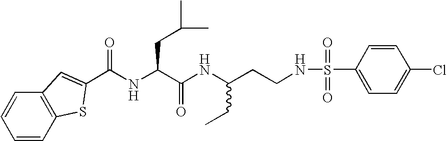 a. 1,1-Dimethylethyl(3-aminopentyl)carbamate

To a pre-cooled solution of (3-amino-1-ethylpropyl)amine (1.27 mL, 10.6 mmol) in CH₂Cl₂ (50 mL) at −40° C. was dropwisely added a pre-cooled solution of 1-({[(1,1-dimethylethyl)oxy]carbonyl}oxy)-2,5-pyrrolidinedione (2.22 g, 10.3 mmol) in CH₂Cl₂ (50 mL) via a cannula) at −40° C. After stirring for 3.5 hr at −40° C., the reaction mixture was warmed up to rt and stirred overnight. The reaction was quenched with 1N aqueous HCl (30 mL) and extracted with CH₂Cl₂ (30 mL×2). The aqueous solution was basified to pH 11-12 with cold 1N aqueous NaOH followed by extraction with EtOAc (50 mL×2). The combined organic solution was dried over K₂CO₃, filtered, and concentrated to provide the title compound which was used for the next reaction without further purification.

b. 1,1-Dimethylethyl (3-{[N-(1-benzothien-2-ylcarbonyl)-L-leucyl]amino}pentyl)carbamate

To a solution of 1,1-dimethylethyl (3-aminopentyl)carbamate (crude from step a, 269 mg, 1.33 mmol) in CH₂Cl₂ (10 mL) were added N-(1-benzothien-2-ylcarbonyl)-L-leucine (388 mg, 1.33 mmol), HOOBt (11 mg, 0.07 mmol), and N-methylmorpholine (0.22 ml, 2.00 mmol). The mixture was stirred several minutes whereupon EDC.HCl (280 mg, 1.46 mmol) was added. The reaction mixture was stirred at RT overnight. The reaction mixture was quenched with cold 1N HCl solution. After extraction with CH₂Cl₂ (15 mL×2), the organic layer was washed with saturated aq. NaHCO₃ solution and brine. The organic solution was dried over MgSO₄, filtered, and concentrated. Purification of the residue by flash column chromatography on silica gel (Biotage, 15%-60% EtOAc/hexane) provided 480 mg of the title compound (76% for step a and step b): LCMS (m/z): 476.4 [M+H]⁺.

c. N-((1S)-1-{[(3-{[(4-chlorophenyl)sulfonyl]amino}-1-ethylpropyl)amino]carbonyl}-3-methylbutyl)-1-benzothiophene-2-carboxamide

To a solution of 1,1-dimethylethyl (3-{[N-(1-benzothien-2-ylcarbonyl)-L-leucyl]amino}pentyl)carbamate (214 mg, 0.45 mmol) in CH₂Cl₂ (2 mL) was added TFA (2 mL) at rt. After 2 hr at rt, the reaction mixture was concentrated and dried under the vacuum pump. The resultant mixture was dissolved in CH₂Cl₂ followed by the addition of 4-chlorobezenesulfonyl chloride (114 mg, 0.54 mmol) and Et₃N (0.26 mL, 1.80 mmol) at rt. After 1 hr at rt, the reaction mixture was concentrated and purified by flash column chromatography on silica gel (Biotage, 15%-45% EtOAc/hexane) to provide 230 mg (93%) of the title compound: LCMS (m/z): 550.2 [M+H]⁺.

### EXAMPLE 130

### Preparation of N-((1S)-1-{[(1-Ethyl-3-{[(2-nitrophenyl)sulfonyl]amino}propyl)amino]carbonyl}-3-methylbutyl)-1-benzothiophene-2-carboxamide

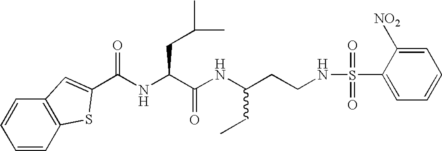

The title compound was prepared following the general procedure of Example 129 except substituting 2-nitrobenzenesulfonyl chloride for 4-chlorobenzenesulfonyl chloride: LCMS (m/z): 561.2 [M+H]⁺.

### EXAMPLE 131

### Preparation of N-{(1S)-1-[({3-[[(4-chlorophenyl)sulfonyl](methyl)amino]-1-ethylpropyl}amino)carbonyl]-3-methylbutyl}-1-benzothiophene-2-carboxamide

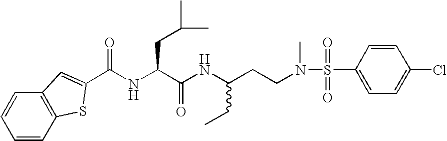

To a solution of N-((1S)-1-{[(3-{[(4-chlorophenyl)sulfonyl]amino}-1-ethylpropyl)amino]carbonyl}-3-methylbutyl)-1-benzothiophene-2-carboxamide of Example 129 (55 mg, 0.1 mmol) in acetone (2 mL) were added K₂CO₃ (21 mg, 0.25 mmol) and MeI 0.03 mL, 0.5 mmol) at rt. After stirring overnight, water (10 mL) was added followed by extraction with EtOAc (10 mL×2). The organic solution was washed with brine and dried over MgSO₄. After concentration, the residue was purified by flash column chromatography on silica gel (Biotage, 0% to 1.5% MeOH/ CH₂Cl₂) to provide 52 mg (92%) of the title compound: LCMS (m/z): 564.2 [M+H]⁺.

### EXAMPLE 132

### Preparation of N-((1S)-1-{[(1-Ethyl-3-{[(2-nitrophenyl)sulfonyl](methyl)amino}propyl)amino]carbonyl}-3-methylbutyl)-1-benzothiophene-2-carboxamide

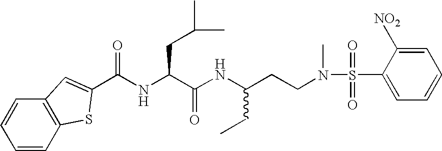

The title compound was prepared following the general procedure of Example 131 except substituting N-((1S)-1-{[(1-ethyl-3-{[(2-nitrophenyl)sulfonyl]amino}propyl)amino]carbonyl}-3-methylbutyl)-1-benzothiophene-2-carboxamide for N-{(1S)-1-[({3-[[(4-chlorophenyl)sulfonyl]amino]-1-ethylpropyl}amino)carbonyl]-3-methylbutyl}-1-benzothiophene-2-carboxamide: LCMS (m/z): 575.2 [M+H]⁺.

### EXAMPLE 133

### Preparation of N-{(1S)-1-[({3-[[(2-cyanophenyl)sulfonyl](methyl)amino]-1-ethylpropyl}amino)carbonyl]-3-methylbutyl}-1-benzothiophene-2-carboxamide

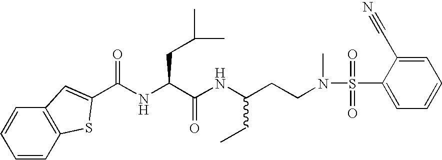

To a solution of N-((1S)-1-{[(1-ethyl-3-{[(2-nitrophenyl)sulfonyl](methyl)amino}propyl)amino]carbonyl}-3-methylbutyl)-1-benzothiophene-2-carboxamide (Example 132, 176 mg, 0.307 mmol) in DMF (1.5 mL) were added PhSH (0.05 mL, 0.46 mmol) and K₂CO₃ (191 mg, 1.38 mmol) at rt. The reaction mixture was stirred at RT for 4 hr. 30 mL of water was added followed by extraction with EtOAc (15 mL×3). After the organic solution was extracted with 1N HCl/ice water (1:1) (10 ml×3), the combined aqueous solution was washed with CH₂Cl₂ twice. The combined aqueous solution was basified to pH 12-13 with 1N NaOH followed by extraction with CH₂Cl₂ three times and washing with saturated NaHCO₃ and brine. The organic layer was dried over K₂CO₃, filtered, and concentrated. To a solution of the above residue in CH₂Cl₂ (5 mL) and saturated aqueous NaHCO₃ (4 ml) at 0° C. was added 2-cyanobenzenesulfonyl chloride (93 mg, 0.46 mmol). The reaction mixture was stirred at 0° C. for 15 min and warmed up to RT, kept stirring for additional 35 min. The residue was diluted with water (15 ml) and extracted twice with dichloromethane. The combined organic solution was washed with brine and dried over MgSO₄, filtered, and concentrated. The residue was purified by flash column chromatography on silica gel (Biotage, 0% to 1% MeOH/CH₂Cl₂) to provide 127 mg of the final compound (75% for two steps): LCMS (m/z): 555.2 [M+H]⁺.

### EXAMPLE 134

### Preparation of N-((1S)-1-{[(3-{[(2-Chloro-4-fluorophenyl)sulfonyl]amino}pentyl)amino]carbonyl}-3-methylbutyl)-1-benzothiophene-2-carboxamide

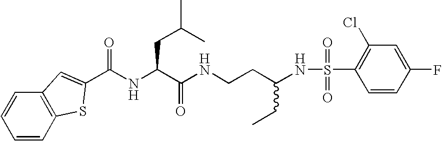 a. 1,1-Dimethylethyl(3-{[(2-chloro-4-fluorophenyl)sulfonyl]amino}pentyl)carbamate

To a solution of 1,1-dimethylethyl (3-aminopentyl)carbamate (Example 129a, 365 mg, 1.8 mmol) in CH₂Cl₂ (10 mL) were added 2-chloro-4-fluorobezenesulfonyl chloride (412 mg, 1.8 mmol) and Et₃N (0.30 mL, 2.17 mmol) at rt. After 2 hr at rt, the reaction mixture was concentrated and purified by flash column chromatography on silica gel (Biotage, 5%-60% EtOAc/hexane) to provide 450 mg (63%) of the title compound: LCMS (m/z): 395.4 [M+H]⁺.

b. N-((1S)-1-{[(3-{[(2-Chloro-4-fluorophenyl)sulfonyl]amino}pentyl)amino]carbonyl}-3-methylbutyl)-1-benzothiophene-2-carboxamide

To a solution of 1,1-dimethylethyl (3-{[(2-chloro-4-fluorophenyl)sulfonyl]amino}pentyl)carbamate (100 mg, 0.25 mmol) in CH₂Cl₂ (2.5 mL) was added 4N HCl in dioxane (0.95 mL, 0.38 mmol) at rt. After 2 hr at rt, the reaction mixture was concentrated and dried further under a vacuum pump. To a solution of the above residue in CH₂Cl₂ (3 mL) were added N-(1-benzothien-2-ylcarbonyl)-L-leucine (80 mg, 0.25 mmol), HOOBt (2 mg, 0.013 mmol), N-methylmorpholine (0.07 ml, 0.625 mmol), and EDC.HCl (55 mg, 0.288 mmol). The reaction mixture was stirred at RT overnight. The reaction mixture was quenched with cold 1N HCl solution. After extraction with CH₂Cl₂ (15 mL×2), the organic layer was washed with saturated aq. NaHCO₃ solution and brine. The organic solution was dried over MgSO₄, filtered, and concentrated. Purification of the residue by flash column chromatography on silica gel (Biotage, 5%-50% EtOAc/hexane) was allowed to separate two diastereomers (36 mg) and (27 mg) with some mixtures: LCMS (m/z): 568.2 [M+H]⁺.

### EXAMPLE 135

### Preparation of N-((1S)-1-{[(3-{[(2,4-Dichlorophenyl)sulfonyl]amino}pentyl)amino]carbonyl}-3-methylbutyl)-1-benzothiophene-2-carboxamide

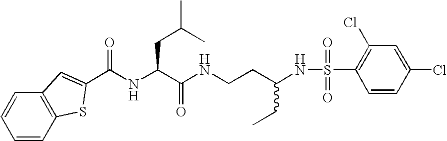

The title compounds were prepared following the general procedure of Example 134 except substituting 2,4-dichlorobenzene sulfonyl chloride for 4-chloro-2-fluorobenzenesulfonyl chloride: LCMS (m/z): 584.2 [M+H]⁺.

### EXAMPLE 136

### Preparation of N-((1S)-1-{[((3R)-3-{[(2-chloro-4-fluorophenyl)sulfonyl]amino}-4-hydroxybutyl)amino]carbonyl}-3-methylbutyl)-1-benzothiophene-2-carboxamide

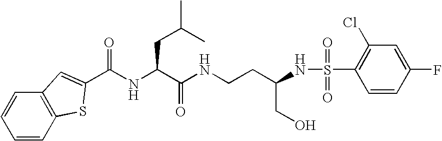 a. 1,1-Dimethylethyl [(3R)-4-hydroxy-3-({[(phenylmethyl)oxy]carbonyl}amino)butyl]carbamate

To a suspension of Z-D-DAB(BOC)-OH DCHA (3.0 g, 5.62 mmol) in THF (28 mL) were added ClCO₂Et (0.59 mL, 6.18 mmol) and NMM (0.65 mL, 5.90 mmol) at −10° C. After stirring vigorously for 10 min between −10 and −20° C., NaBH₄ (640 mg, 16.86 mmol) was added to the reaction mixture followed by the slow addition of MeOH (56 mL). After stirring for 10 min at 0° C., the reaction mixture was concentrated. The residue was diluted with 30 mL of CH₂Cl₂ and 30 mL of H₂O and neutralized with 1N aq. HCl solution. After extraction with CH₂Cl₂ (20 mL×2), the organic solution was washed with saturated aq. NaHCO₃ and brine, filtered, and concentrated. The residue was purified by flash column chromatography on silica gel (Biotage, 10% to 100% EtOAc/hexane) to provide 1.58 g (83%) of the final compound: LCMS (m/z): 339.2 [M+H]⁺.

b. 1,1-dimethylethyl ((3R)-3-{[(2-chloro-4-fluorophenyl)sulfonyl]amino}-4-hydroxybutyl)carbamate

To a solution of 1,1-Dimethylethyl [(3R)-4-hydroxy-3-({[(phenylmethyl)oxy]carbonyl}amino)butyl]carbamate (303 mg, 0.896 mmol) in ethanol (2.0 ml) was added Pd/C (10% w/w, 57 mg, 0.054 mmol). Using a septum, hydrogen gas was allowed to enter the reaction mixture from a balloon. After stirring for 2 h, the reaction mixture was filtered through celite then concentrated under reduced pressure to obtain a residue that was used without further purification. To a solution of the residue in dichloromethane (5 mL) was added saturated NaHCO₃ (aq, 3 mL) followed by 2-chloro-4-fluorobenzenesulfonyl chloride (246.3 mg, 1.08 mmol). After stirring the reaction mixture overnight the organic layer was separated, dried over MgSO₄, filtered and concentrated under reduced pressure. The residue was purified by flash column chromatography on silica gel (Biotage, 0% to 2.50% MeOH/CH₂Cl₂) to provide 242 mg (68%, 2 steps) of the title compound: LCMS (m/z): 297.0 [(M−100)+H]⁺.

c. N-((1S)-1-{[((3R)-3-{[(2-chloro-4-fluorophenyl)sulfonyl]amino}-4-hydroxybutyl)amino]carbonyl}-3-methylbutyl)-1-benzothiophene-2-carboxamide (Non-preferred Name)

To a solution of 1,1-dimethylethyl ((3R)-3-{[(2-chloro-4-fluorophenyl)sulfonyl]amino}-4-hydroxybutyl)carbamate (237 mg, 0.598 mmol) in methanol (4 mL), was added a 4 N solution of HCl in dioxane (1.5 mL, 5.98 mmol) and stirring was continued for 2 h. The reaction mixture was then concentrated under reduced pressure and azeotroped three times using toluene then vacuum dried. To a solution of the residue in dichloromethane (6 mL) were added N-(1-benzothien-2-ylcarbonyl)-L-leucine (191.4 mg, 0.658 mmol), HOOBt (2.44 mg, 0.015 mmol), N-methylmorpholine (0.26 ml, 2.39 mmol), and EDC.HCl (126.3 mg, 0.658 mmol). The reaction mixture was stirred at RT overnight, then quenched with 10% (aq) citric acid (10 mL). The organic layer was washed once with saturated NaHCO₃ and brine, dried over MgSO₄, then concentrated under reduced pressure. The residue obtained was purified by flash column chromatography on silica gel (Biotage, 0% to 2.0% MeOH/CH₂Cl₂) to provide 193 mg (57%, 2 steps) of the title compound: LCMS (m/z): 570.2 [M+H]⁺.

### EXAMPLE 137

### Preparation of N-((1S)-1-{[((3S)-3-{[(2-chloro-4-fluorophenyl)sulfonyl]amino}-4-hydroxybutyl)amino]carbonyl}-3-methylbutyl)-1-benzothiophene-2-carboxamide

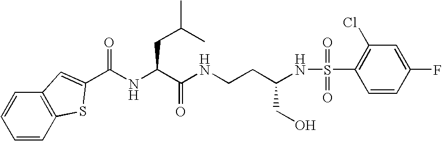

The title compounds were prepared following the general procedure of Example 136 except substituting Z-DAB(BOC)-OH DCHA for Z-D-DAB(BOC)-OH DCHA: LCMS (m/z): 570.2 [M+H]⁺.

### EXAMPLE 138

### Preparation of Methyl (2R)-4-{[N-(1-benzothien-2-ylcarbonyl)-L-leucyl]amino}-2-{[(2-chloro-4-fluorophenyl)sulfonyl]amino}butanoate

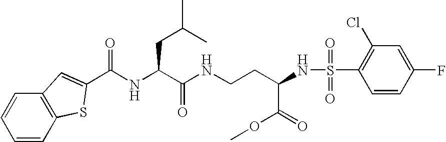

The title compound was prepared following the procedure of Example 137 except for the following change in step 137a. The carboxylic acid group of Z-D-DAB(BOC)-OH.DCHA was converted to a methyl ester. The methyl ester was obtained quantitatively using trimethylsilyl diazomethane (3 equiv) in toluene:methanol (2:1) at 0° C.: LCMS (m/z): 599.2 [M+H]⁺

### EXAMPLE 139

### Preparation of N-((1S)-1-{[((3S)-3-{[(2-chloro-4-fluorophenyl)sulfonyl]amino}butyl)amino]carbonyl}-3-methylbutyl)-1-benzothiophene-2-carboxamide

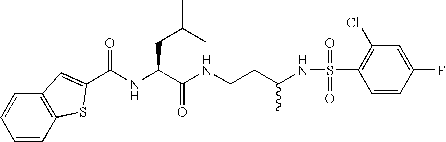 a. 1,1-Dimethylethyl[(3R)-4-iodo-3({[(phenylmethyl)oxy]carbonyl}amino)butyl]carbamate

To a suspension of PPh₃ (210 mg, 0.8 mmol) and imidazole (109 mg, 1.6 mmol) in CH₂Cl₂ at 0° C. was added iodine (203 mg, 0.8 mmol). The reaction mixture turned to a deep yellow suspension. After stirring for 15 min at 0° C., 1,1-dimethylethyl [(3R)-4-hydroxy-3-({[(phenylmethyl)oxy]carbonyl}amino)butyl]carbamate (step a in Example 8) (138 mg, 0.4 mmol) was added to the above reaction mixture. After 5 min, the reaction mixture was warmed up to RT and stirred for 3 hr. After concentration, the residue was purified by flash column chromatography on silica gel (Biotage, 5%-45% EtOAc/hexane) to provide 155 mg of the title product (87%).

b. 1,1-Dimethylethyl phenylmethyl 1,3-butanediylbiscarbamate

To the solution of 1,1-dimethylethyl [(3R)-4-iodo-3-({[(phenylmethyl)oxy]carbonyl}amino)butyl]carbamate (0.33 g, 0.74 mmol) in THF (8 ml) was added N-selectride(0.95 ml, 1.0 M in THF) at −20° C. The reaction mixture was stirred at this temperature for 20 min and −5° C. for 1 hr. The reaction was quenched with 0.1 N aq. Na₂S₂O₃ (10 ml) and saturated aq. NaHCO₃ (10 ml) solution. After extraction with EtOAc (10 ml×3), the combined organic solution was washed with brine and dried over MgSO₄, filtered and concentrated. The residue was purified by flash column chromatography on silica gel (Biotage, 5%-50% EtOAc/hexane) to provide 0.18 g of the title product (79%).

c. 1,1-Dimethylethyl (3-aminobutyl)carbamate

To the solution of 1,1-dimethylethyl phenylmethyl 1,3-butanediylbiscarbamate (0.19 g, 0.59 mmol) in 5 ml of EtOH was added 30 mg of 10% Pd on carbon under N₂. The reaction mixture was stirred under H₂ atmosphere (1 atm) at room temperature for 5 hours. The solution was filtered and concentrated to give 0.10 g of the title product (90%).

d. 1,1-Dimethylethyl(3-{[(2-chloro-4-fluorophenyl)sulfonyl]amino}butyl)carbamate

To a solution of 1,1-dimethylethyl (3-aminobutyl)carbamate (0.10 g, 0.53 mmol) in CH₂Cl₂ (5 mL) were added 2-chloro-4-fluorobezenesulfonyl chloride (0.15 g, 0.64 mmol) and Et₃N (0.22 mL, 1.60 mmol) at rt. After 2 hr at rt, the reaction mixture was concentrated and purified by flash column chromatography on silica gel (Biotage, 5%-40% EtOAc/hexane) to provide 0.15 g (74%) of the title compound.

e. N-(3-Amino-1-methylpropyl)-2-chloro-4-fluorobenzenesulfonamide

To a solution of 1,1-dimethylethyl (3-{[(2-chloro-4-fluorophenyl)sulfonyl]amino}butyl)carbamate (150 mg, 0.44 mmol) in MeOH (2.0 mL) was added 4N HCl in dioxane (2 mL, 8.0 mmol) at rt. After 2 hr at rt, the reaction mixture was concentrated and dried further under a vacuum pump. To a solution of the above residue in CH₂Cl₂ (5 mL) were added N-(1-benzothien-2-ylcarbonyl)-L-leucine (130 mg, 0.44 mmol), HOOBt (2 mg, 0.013 mmol), N-methylmorpholine (0.20 ml, 1.76 mmol), and EDC.HCl (91 mg, 0.48 mmol). The reaction mixture was stirred at RT overnight. The reaction mixture was quenched with water. After extraction with CH₂Cl₂ (15 mL×2), the combined organic solution was washed with saturated aq. NaHCO₃ solution and brine. The organic solution was dried over MgSO₄, filtered, and concentrated. Purification of the residue by flash column chromatography on silica gel (Biotage, 5%-50% EtOAc/hexane) was allowed to separate two diastereomers (91 mg) and (30 mg) with some mixtures: LCMS (m/z): 554.2 [M+H]⁺.

### EXAMPLE 140

### Preparation of 5-{[N-(1-Benzothien-2-ylcarbonyl)-L-leucyl]amino}-3-{[(2-chloro-4-fluorophenylsulfonyl]amino}-1,2,3,5-tetradeoxy-D-erythro-pentitol

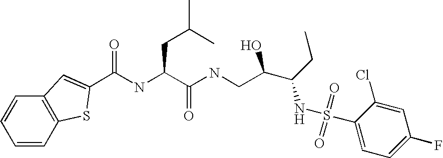 a. Bis(1,1-dimethylethyl)(2E)-2-penten-1-ylimidodicarbonate

To a solution of trans-2-penten-1-ol (1.70 g, 19.7 mmol) in THF (57 mL) at 0° C. was added bis(1,1-dimethylethyl)imidodicarbonate (5.58 g, 25.7 mmol), PPh₃ (6.7 g, 25.7 mmol) and DIAD (4.98 mL, 25.7 mmol). The reaction was stirred overnight at ambient temperature then concentrated under reduced pressure. The residue was purified by silica gel chromatography (Biotage, 0% to 8% EtOAc/hexane) to yield the title compound as a clear oil (2.1 g, 48%): ¹H NMR (400 MHz, CDCl₃) δ 1.0 (t, J=7.6 Hz, 3H), 1.50 (s, 18H), 2.05 (m, 2H), 4.10 (m, 2H), 5.45 (m, 1H), 5.65 (m, 1H).

b. 1,1-Dimethylethyl (5R)-5-[(1R)-1-hydroxypropyl]-2-oxo-1,3-oxazolidine-3-carboxylate

To a mixture of AD-mix-(β) (7.11 g) methanesulfonamide (0.487 g), and potassium osmate dihydrate (15 mg) in t-BuOH:H₂O (30 mL:30 mL) at 0° C. was added a solution of bis(1,1-dimethylethyl)(2E)-2-penten-1-ylimidodicarbonate (1.46 g, 5.12 mmol) in t-BuOH (4.0 mL). The reaction mixture was stirred at 0° C. until starting material was mostly consumed as monitored by TLC. The reaction was quenched by adding 7.6 g of Na₂SO₃ and stirring for 1 hr at RT. Finally, the reaction mixture was extracted with ethyl acetate (150 mL×3) and dried over MgSO₄ then concentrated under reduced pressure. The residue was purified by silica gel chromatography (Biotage, 5% to 45%, EtOAc/hexane) to yield the title compound as a white solid (0.73 g, 58%); LCMS (m/z): 146.0 [(M+H)−100]⁺.

c. 1,1-Dimethylethyl (5R)-5-[(1S)-1-azidopropyl]-2-oxo-1,3-oxazolidine-3-carboxylate

To a solution of 1,1-dimethylethyl (5R)-5-[(1R)-1-hydroxypropyl]-2-oxo-1,3-oxazolidine-3-carboxylate (180 mg, 0.735 mmol) in THF (5 mL) at 0° C. was added DPPA (0.175 mL, 0.808 mmol), PPh₃ (212 mg, 0.808 mmol), and DEAD (0.127 mL, 0.808 mmol).

The reaction mixture was stirred overnight at ambient temperature then concentrated under reduced pressure. The residue was purified by silica gel chromatography (Biotage, 5% to 15% EtOAc/hexane) to yield the title compound (135 mg, 68%): LCMS (m/z): 271.2 [M+H]⁺.

d. 3-Azido-1,2,3,5-tetradeoxy-5-({[(1,1-dimethylethyl)oxy]carbonyl}amino)-D-erythro-pentitol

To a solution of 1,1-dimethylethyl (5R)-5-[(1S)-1-azidopropyl]-2-oxo-1,3-oxazolidine-3-carboxylate (93 mg, 0.344 mmol) in methanol (3 mL) was added cesium carbonate (22.5 mg, 0.069 mmol). The reaction mixture was stirred for 3 hr then quenched with 10% (v/v) aq. citric acid, extracted with ethyl acetate (60 mL×2), dried over MgSO₄, filtered, and concentrated under reduced pressure. The residue was purified by silica gel chromatography (Biotage, 15% to 20%, EtOAc/hexane) to yield the title compound as a clear oil (79 mg, 90%): LCMS (m/z): 245.2 [M+H]⁺.

e. 3-Amino-1,2,3,5-tetradeoxy-5-({[(1,1-dimethylethyl)oxy]carbonyl}amino)-D-erythro-pentitol

To a solution of 3-azido-1,2,3,5-tetradeoxy-5-({[(1,1-dimethylethyl)oxy]carbonyl}amino)-D-erythro-pentitol (79 mg, 0.324 mmol) in methanol (1.0 mL) was added 10% (w/w) Pd/C (34.3 mg, 0.032 mmol). Hydrogen gas under balloon pressure was allowed to enter the reaction mixture for 1 hr. The reaction mixture was then filtered through a plug of celite and concentrated under reduced pressure. The residue was used in the next step without further purification: LCMS (m/z): 219.2 [M+H]⁺.

f. 3-{[(2-Chloro-4-fluorophenyl)sulfonyl]amino}-1,2,3,5-tetradeoxy-5-({[(1,1-dimethylethyl)oxy]carbonyl}amino)-D-erythro-pentitol

To a solution of 3-amino-1,2,3,5-tetradeoxy-5-({[(1,1-dimethylethyl)oxy]carbonyl}amino)-D-erythro-pentitol (68 mg, 0.312 mmol) in saturated aq. NaHCO₃:CH₂Cl₂ (1.5 mL:3.0 mL) at rt was added 2-chloro-4-fluorobenzenesulfonyl chloride (92.9 mg, 0.406 mmol). After stirring overnight at RT, the organic layer was separated, dried over MgSO₄ then concentrated under reduced pressure. The residue was purified by silica gel chromatography (Biotage, 15% to 40%, EtOAc/hexane) to yield the title compound (85 mg, 66%): LCMS (m/z): 411.2 [M+H]⁺.

g. 5-{[N-(1-Benzothien-2-ylcarbonyl)-L-leucyl]amino}-3-{[(2-chloro-4-fluorophenyl)sulfonyl]amino}-1,2,3,5-tetradeoxy-D-erythro-pentitol

To a solution of 3-{[(2-chloro-4-fluorophenyl)sulfonyl]amino}-1,2,3,5-tetradeoxy-5-({[(1,1-dimethylethyl)oxy]carbonyl}amino)-D-erythro-pentitol (83 mg, 0.202 mmol) in methanol (2.0 mL) was added a solution of 4 N HCl in dioxane (0.51 mL, 2.02 mmol). After stirring for 2.5 hr, the solvent was evaporated and the mixture azeotroped with toluene. To a suspension of the resultant residue, 5-amino-3-{[(2-chloro-4-fluorophenyl)sulfonyl]amino}-1,2,3,5-tetradeoxy-D-erythro-pentitol hydrochloride in dichloromethane (3 mL) was added N-(1-benzothien-2-ylcarbonyl)-L-leucine (64.6 mg, 0.222 mmol), HOOBt (0.82 mg, 0.005 mmol), NMM (0.089 mL, 0.808 mmol), and EDC.HCl (42.6 mg, 0.222 mmol). The reaction mixture was stirred overnight then quenched with 10% (v/v) aq. citric acid. The organic layer was separated, washed with brine then dried over MgSO₄ and concentrated. The residue was purified by chromatography to yield the title compound (55 mg, 47%): LCMS (m/z): 584.2 [M+H]⁺.

### EXAMPLE 141

### Preparation of N-{(1S)-1-[({3-[[(2-Cyanophenyl)sulfonyl](methyl)amino]propyl}amino)carbonyl]-3-methylbutyl}-3-methyl-1-benzothiophene-2-carboxamide

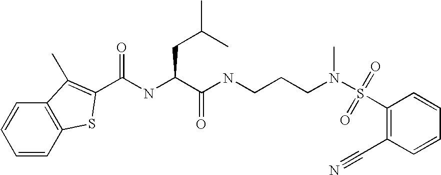

The title compound was prepared following the general procedure of Example 67 except for the substitution of 3-methyl-1-benzothiophene-2-carboxylic acid for thieno[3,2-b]thiophene-2-carboxylic acid: LCMS (m/z): 541 [M+H]⁺.

### EXAMPLE 142

### Preparation of 5-Chloro-N-{(1S)-1-[({3-[[(2-cyanophenyl)sulfonyl](methyl)amino]propyl}amino)carbonyl]-3-methylbutyl}-3-methyl-1-benzothiophene-2-carboxamide

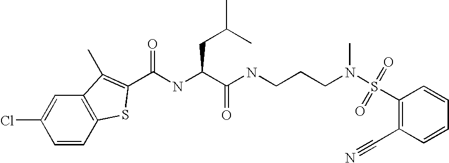

The title compound was prepared following the general procedure of Example 67 except for the substitution of 5-chloro-3-methyl-1-benzothiophene-2-carboxylic acid for thieno[3,2-b]thiophene-2-carboxylic acid: LCMS (m/z): 575 [M+H]⁺.

### EXAMPLE 143

### Preparation of N¹-{3-[[(2-Cyanophenyl)sulfonyl](methyl)amino]propyl}-N²-(2-thienylcarbonyl)-L-leucinamide

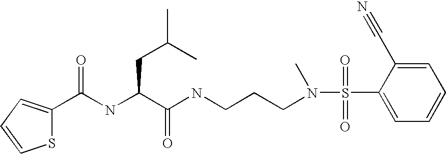

The title compound was prepared following the general procedure of Example 67 except for the substitution of 2-thiophenecarboxylic acid for thieno[3,2-b]thiophene-2-carboxylic acid: LCMS (m/z): 477.2 [M+H]⁺.

### EXAMPLE 144

### Preparation of N-[(1S)-2-({3-[[(2-Cyanophenyl)sulfonyl](methyl)amino]propyl}amino)-1-(cyclopropylmethyl)-2-oxoethyl]-1-benzothiophene-2-carboxamide

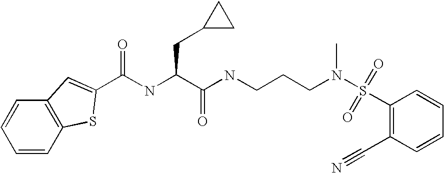

The title compound was prepared following the general procedure of Example 74 except for substituting 3-cyclopropyl-L-alanine for 3-cyclohexyl-L-alanine: LCMS (m/z): 525 [M+H]⁺.

### EXAMPLE 145

### Preparation of 1,1-Dimethylethyl 4-[(2S)-2-[(1-benzothien-2-ylcarbonyl)amino]-3-({3-[[(2-cyanophenyl)sulfonyl](methyl)amino]propyl}amino)-3-oxopropyl]-1-piperidinecarboxylate

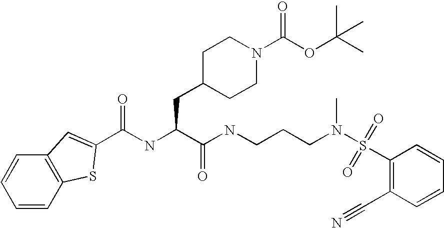

The title compound was prepared following the general procedure of Example 74 except for substituting 3-(1-{[(1,1-dimethylethyl)oxy]carbonyl}-4-piperidinyl)-N-{[(9H-fluoren-9-ylmethyl)oxy]carbonyl}-L-alanine for 3-cyclohexyl-L-alanine: LCMS (m/z): 668 [M+H]⁺.

### EXAMPLE 146

### Preparation of N-[2-({3-[[(2-Cyanophenyl)sulfonyl](methyl)amino]propyl}amino)-2-oxo-1-(tetrahydro-2H-pyran-4-ylmethyl)ethyl]-1-benzothiophene-2-carboxamide

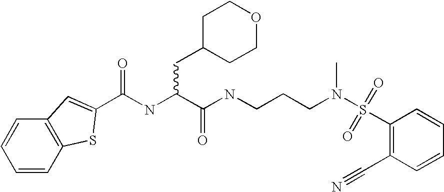

The title compound was prepared following the general procedure of Example 74 except for substituting 3-(tetrahydro-2H-pyran-4-yl)-L-alanine for 3-cyclohexyl-L-alanine: LCMS (m/z): 569 [M+H]⁺.

### EXAMPLE 147

### Preparation of N-((1S)-1-{[(3-{[(2-cyanophenyl)sulfonyl][2-(dimethylamino)ethyl]amino}propyl)amino]carbonyl}-3-methylbutyl)-1-benzothiophene-2-carboxamide

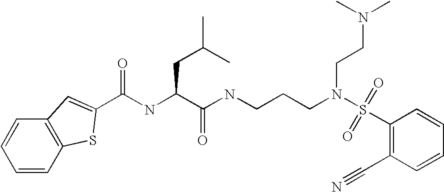

The title compound was prepared following the procedure of Example 80 except for the substitution of N,N-dimethyl-1,2-ethanediamine for cyclopropylamine. LCMS (m/z): 584 [M+H]⁺.

### EXAMPLE 148

### Preparation of N-((1S)-1-{[(3-{[(2-cyanophenyl)sulfonyl][2-(methyloxy)ethyl]amino}propyl)amino]carbonyl}-3-methylbutyl)-1-benzothiophene-2-carboxamide

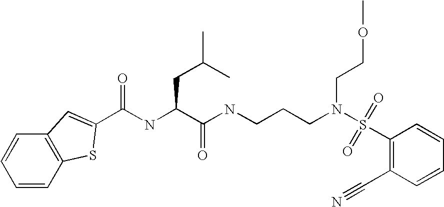

The title compound was prepared following the procedure of Example 80 except for the substitution of 2-methoxy-ethylamine for cyclopropylamine. LCMS (m/z): 571 [M+H]⁺.

### EXAMPLE 149

### Preparation of N-{(1S)-1-[({3-[[(2-cyanophenyl)sulfonyl](methyloxy)amino]propyl}amino)carbonyl]-3-methylbutyl}-1-benzothiophene-2-carboxamide

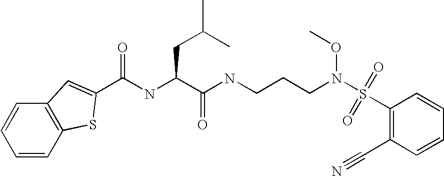

The title compound was prepared following the procedure of Example 80 except for the substitution of methoxylamine for cyclopropylamine. LCMS (m/z): 543 [M+H]⁺.

### EXAMPLE 150

### Preparation of N-{(1S)-1-[({3-[[(2-cyanophenyl)sulfonyl](2-pyridinylmethyl)amino]propyl}amino)carbonyl]-3-methylbutyl}-1-benzothiophene-2-carboxamide

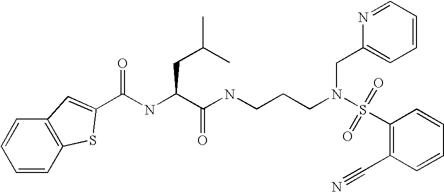

The title compound was prepared following the procedure of Example 80 except for the substitution of 2-(aminomethyl)-pyridine for cyclopropylamine. LCMS (m/z): 604 [M+H]⁺.

### EXAMPLE 151

### Preparation of N-((1S)-1-{[(3-{butyl[(2-cyanophenyl)sulfonyl]amino}propyl)amino]carbonyl}-3-methylbutyl)-1-benzothiophene-2-carboxamide

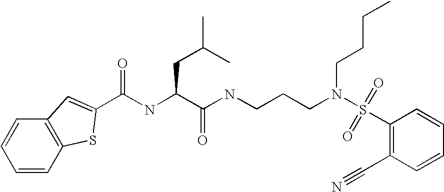

The title compound was prepared following the procedure of Example 80 except for the substitution of butylamine for cyclopropylamine. LCMS (m/z): 569 [M+H]⁺.

### EXAMPLE 152

### Preparation of N-((1S)-1-{[(3-{butyl[(2,4-dichlorophenyl)sulfonyl]amino}propyl)amino]carbonyl}-3-methylbutyl)-1-benzothiophene-2-carboxamide

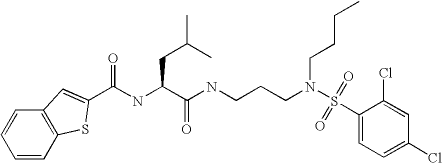

The title compound was prepared following the procedure of Example 151 except for the substitution of 2,4-dichlorobenzene sulfonyl chloride in place of 2-cyanobenzene sulfonyl chloride. NMR H¹: (δ) 0.80 (t, 3H), 1.0 (m, 6H), 1.15 (m, 2H), 1.45 (m, 2H), 1.65-1.90 (m, 5H), 3.15 (m, 2H), 3.40 (m, 4H), 4.65 (m, 1H), 6.65 (m, 1H), 6.80 (m, 1H), 7.25-7.45 (m, 4H), 7.80 (m, 3H), 7.95 (m, 1H).

### EXAMPLE 153

### Preparation of N-[(1S)-2-({3-[[(2-cyanophenyl)sulfonyl](cyclopropyl)amino]propyl}amino)-1-(cyclopentylmethyl)-2-oxoethyl]-1-benzothiophene-2-carboxamide

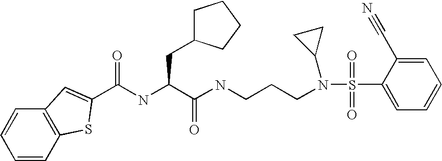

The title compound was prepared following the procedure of Example 80 except for the substitution of N-(1-benzothien-2-ylcarbonyl)-3-cyclopentyl-L-alanine for N-(1-benzothien-2-ylcarbonyl)-L-leucine in the first step: LCMS (m/z): 579.0 [M+H]⁺.

### EXAMPLE 154

### Preparation of N-((1S)-1-{[(3-{[(2-chloro-4-fluorophenylsulfonyl]amino}-2,2-dimethylpropyl)amino]carbonyl}-3-methylbutyl)-1-benzothiophene-2-carboxamide

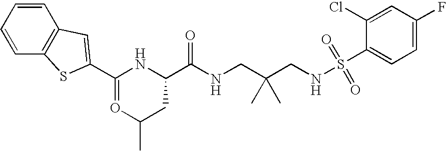

The title compound was prepared following the general procedure of Example 96 except substituting 1-dimethylethyl (3-aminopropyl)carbamate for 1,1-dimethylethyl (3-amino-2,2-dimethylpropyl)carbamate: LCMS (m/z): 568.4 [MH]⁺.

### EXAMPLE 155

### Preparation of N-{(1S)-1-[({(2R)-3-[[(2-chlorophenyl)sulfonyl](methyl)amino]-2-hydroxypropyl}amino)carbonyl]-3-methylbutyl}-1-benzothiophene-2-carboxamide

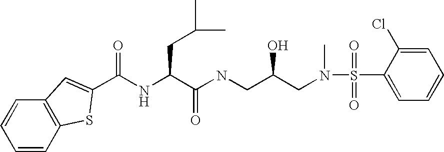

The title compound was prepared following the general procedure of Example 103 except substituting 2-cyanobenzenesulfonyl chloride for 2-chlorobenzenesulfonyl chloride: LCMS (m/z): 552.2 [M+H]⁺.

### EXAMPLE 156

### Preparation of N-{(1S)-1-[({(2R)-3-[[(4-chlorophenyl)sulfonyl](methyl)amino]-2-hydroxypropyl}amino)carbonyl]-3-methylbutyl}-1-benzothiophene-2-carboxamide

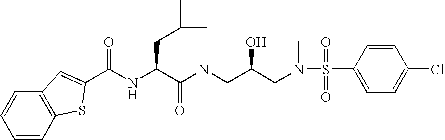

The title compound was prepared following the general procedure of Example 103 except substituting 2-cyanobenzenesulfonyl chloride for 4-chlorobenzenesulfonyl chloride: LCMS (m/z): 552.2 [M+H]⁺.

### EXAMPLE 157

### Preparation of N-{(1S)-1-[({(2R)-3-[[(2-cyanophenyl)sulfonyl](methyl)amino]-2-fluoropropyl}amino)carbonyl]-3-methylbutyl}-1-benzothiophene-2-carboxamide

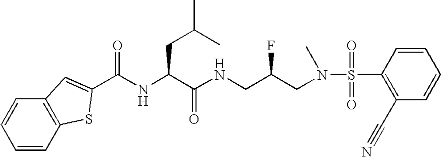

To a solution of N-{(1S)-1-[({(2S)-3-[[(2-cyanophenyl)sulfonyl](methyl)amino]-2-hydroxypropyl}amino)carbonyl]-3-methylbutyl}-1-benzothiophene-2-carboxamide (100 mg, 0.185 mmol) THF (1.5 mL) at rt, was added PBSF (0.080 mL, 0.443 mmol), triethylamine trihydrofluoride (0.072 mL, 0.443 mmol), and triethylamine (0.180 mL, 1.29 mmol). After stirring 2 days at rt, the reaction mixture was diluted with dichloromethane, washed with saturated aq. NaHCO₃, and brine. The organic layer was dried over MgSO₄, concentrated, and the residue purified using silica gel chromatography (Biotage, 0% to 1.0%, MeOH/CH₂Cl₂) to provide the title compound (56 mg, 56%): LCMS (m/z): 543.2 [M+H]⁺.

### EXAMPLE 158

### Preparation of N-{(1S)-1-[({(2R)-3-[[(2,4-dichlorophenyl)sulfonyl](methyl)amino]-2-fluoropropyl}amino)carbonyl]-3-methylbutyl}-1-benzothiophene-2-carboxamide

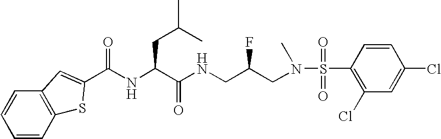

The title compound was prepared following the procedure of Example 157 except for the use of 2,4-dichlorobenzenesulfonyl chloride in place of 2-cyanobenzenesulfonyl chloride: LCMS (m/z): 588.0 [M+H]⁺

### EXAMPLE 159

### Preparation of N-{(1S)-1-[({(2R)-3-[[(2-chloro-4-fluorophenyl)sulfonyl](methyl)amino]-2-fluoropropyl}amino)carbonyl]-3-methylbutyl}-1-benzothiophene-2-carboxamide

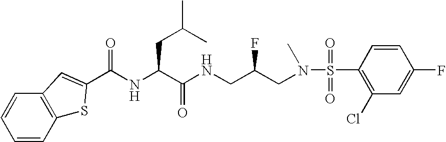

The title compound was prepared following the procedure of Example 157 except for the use of 2-chloro-4-fluorobenzenesulfonyl chloride in place of 2-cyanobenzenesulfonyl chloride: LCMS (m/z): 573.0 [M+H]⁺

To a suspension of Z-D-DAB(BOC)-OH DCHA (3.0 g, 5.62 mmol) in THF (28 mL) were added ClCO₂Et (0.59 mL, 6.18 mmol) and NMM (0.65 mL, 5.90 mmol) at −10° C.

After stirring vigorously for 10 min between −10 and −20° C., NaBH₄ (640 mg, 16.86 mmol) was added to the reaction mixture followed by the slow addition of MeOH (56 mL). After stirring for 10 min at 0° C., the reaction mixture was concentrated. The residue was diluted with 30 mL of CH₂Cl₂ and 30 mL of H₂O and neutralized with 1N aq. HCl solution. After extraction with CH₂Cl₂ (20 mL×2), the organic solution was washed with saturated aq. NaHCO₃ and brine, filtered, and concentrated. The residue was purified by flash column chromatography on silica gel (Biotage, 10% to 100% EtOAc/hexane) to provide 1.58 g (83%) of the final compound: LCMS (m/z): 339.2 [M+H]⁺.

b) Phenylmethyl (2R)-4-({[(1,1-dimethylethyl)oxy]carbonyl}amino)-2-[(1,3-dioxo-1,3-dihydro-2H-isoindol-2-yl)methyl]butanoate

To a solution of 1,1-Dimethylethyl [(3R)-4-hydroxy-3-({[(phenylmethyl)oxy]carbonyl}amino)butyl]carbamate (462 mg, 1.37 mmol) in THF (4.0 mL) at 0° C. was added phthalimide (221 mg, 1.50 mmol), triphenylphosphine (449 mg, 1.71 mmol), and DEAD (0.270 ml, 1.71 mmol). After stirring overnight at RT, the mixture was concentrated under reduced pressure and the residue was purified by flash silica gel chromatography (Biotage, 10% to 50%, EtOAc/Hexanes) to yield a (partly purified) title compound (420 mg, <65%): LCMS (m/z): 468.2 [M+H]⁺.

c) Phenylmethyl {(1R)-3-{[N-(1-benzothien-2-ylcarbonyl)-L-leucyl]amino}-1-[(1,3-dioxo-1,3-dihydro-2H-isoindol-2-yl)methyl]propyl}carbamate

To a solution of phenylmethyl (2R)-4-({[(1,1-dimethylethyl)oxy]carbonyl}amino)-2-[(1,3-dioxo-1,3-dihydro-2H-isoindol-2-yl)methyl]butanoate (403 mg, 0.863 mmol) in MeOH:CH₂Cl₂ (6 mL, 2:1) was added 4N HCl in dioxane (2.15 mL, 8.63 mmol). After 2 hr at RT, the reaction mixture was concentrated under reduced pressure and azeotroped with toluene. To a solution of the residue (223 mg, 0.553 mmol) in dichloromethane was added N-(1-benzothien-2-ylcarbonyl)-L-leucine (193 mg, 0.663 mmol), HOOBt (2.3 mg, 0.014 mmol), NMM (0.243 mL, 2.21 mmol), and EDC.HCl (127.3 mg, 0.663 mmol). After stirring overnight at RT, the reaction mixture was washed with saturated aq. NaHCO₃ and brine, dried over MgSO₄, then concentrated under reduced pressure. The residue was purified by flash silica gel chromatography (Biotage, 0% to 2.0% MeOH/DCM) to yield the title compound (305 mg, 86%): LCMS (m/z): 641.4 [M+H]⁺.

d) N-[(1S)-1-({[(3R)-3-{[(2-Chloro-4-fluorophenyl)sulfonyl]amino}-4-(1,3-dioxo-1,3-dihydro-2H-isoindol-2-yl)butyl]amino}carbonyl)-3-methylbutyl]-1-benzothiophene-2-carboxamide

To a solution of phenylmethyl {(1R)-3-{[N-(1-benzothien-2-ylcarbonyl)-L-leucyl]amino}-1-[(1,3-dioxo-1,3-dihydro-2H-isoindol-2-yl)methyl]propyl}carbamate (271 mg, 0.423 mmol) in dichloromethane (8.0 mL) at 0° C. was slowly added a 1 M solution of BBr₃ in dichloromethane (2.12 mL, 2.12 mmol). The reaction mixture was allowed to warm to rt and stirred for about 2 h and a suspension was formed. The solid was collected by filtration and used without further purification (178 mg, 83%). To a suspension of the solid (178 mg, 0.352 mmol) in dichloromethane was added 2-chloro-4-fluorobenzenesulfonyl chloride (112.8 mg, 0.492 mmol), and triethylamine (0.147 mL, 1.06 mmol). After stirring at rt for 3 h, the mixture was concentrated and purified by silica gel chromatography (Biotage, 0% to 2.0% MeOH/CH₂Cl₂) to provide the title compound (100 mg, 40%): LCMS (m/z): 699.2 [M+H]⁺.

e) N-((1S)-1-{[((3R)-4-(Acetylamino)-3-{[(2-chloro-4-fluorophenyl)sulfonyl]amino}butyl)amino]carbonyl}-3-methylbutyl)-1-benzothiophene-2-carboxamide

To a solution of N-[(1S)-1-({[(3R)-3-{[(2-chloro-4-fluorophenyl)sulfonyl]amino}-4-(1,3-dioxo-1,3-dihydro-2H-isoindol-2-yl)butyl]amino}carbonyl)-3-methylbutyl]-1-benzothiophene-2-carboxamide (80 mg, 0.115 mmol) in ethanol (2.0 mL) was added hydrazine (0.018 mL, 0.573 mmol) and the mixture was stirred for 2 hr at RT. The solid was removed by filtration and the filtrate was concentrated under reduced pressure to obtain a solid residue that was used without further purification. To a solution of the residue (50 mg, 0.088 mmol) in dichloromethane was added triethyamine (0.037 mL, 0.264 mmol) and acetic anhydride (0.012 mL, 0.132 mmol). After stirring overnight at RT, the mixture was concentrated under reduced pressure and purified by silica gel chromatography ((Biotage, 0% to 3.0% MeOH/CH₂Cl₂) to provide the title compound (36 mg, 67%): LCMS (m/z): 611.2 [M+H]⁺.

### EXAMPLE 159

### Tablet Composition

The sucrose, calcium sulfate dihydrate and a TRPV4 agonist as shown in Table 3 below, are mixed and granulated in the proportions shown with a 10% gelatin solution. The wet granules are screened, dried, mixed with the starch, talc and stearic acid, screened and compressed into a tablet.

TABLE 3

| INGREDIENTS | AMOUNTS |
|---|---|
| N—{(1S)-1-[({3-[[(2-chloro-4- | 20 mg |
| cyanophenyl)sulfonyl](methyl)amino]prop- | |
| yl}amino)carbonyl]-3-methylbutyl}-1- | |
| benzothiophene-2-carboxamide; | |
| calcium sulfate dihydrate | 30 mg |
| sucrose | 4 mg |
| starch | 2 mg |
| talc | 1 mg |
| stearic acid | 0.5 mg |

What is claimed is:

1. A compound of formula I 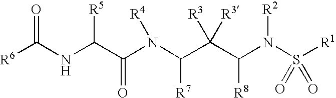
or a pharmaceutically acceptable salt thereof, or a solvate thereof, or a combination thereof, wherein:
R¹ is phenyl, thienyl, furanyl, benzoxadiazolyl, imidazo[2,1-b][1,3]thiazolyl, C₃-C₇ cycloalkyl-C₁-C₄ alkylenyl, C₃-C₇ cycloalkyloxy-C₁-C₄ alkylenyl, or N-ethenyl-tetrahydroindolyl, wherein R¹ is optionally substituted with one or more substituents selected from the group consisting of C₁-C₆ alkyl, C₁-C₆ alkylsulfonyl, [(methylamino)carbonyl]amino, cyano, nitro, trifluoromethyl, trifluoromethoxy, carboC₁-C₆alkyloxy, and halo;
R² is H, C₁-C₆ alkyl, haloC₁-C₆ alkyl, diC₁-C₆ alkylamino-C₁-C₆ alkylenyl, C₁-C₆ alkyloxy-C₁-C₆ alkylenyl, C₁-C₆ alkyloxy, pyridinyl-C₁-C₆ alkylenyl, C₃-C₇ cycloalkyl, or tetrahydropyranyl;
R³ is H, hydroxy, —O—C₁-C₆ alkyl, —SH, —S—C₁-C₆ alkyl, amino, C₁-C₄ alkylamino, propenyloxy, or halo;
R^{3′} is H or C₁-C₆ alkyl, or R^{3′} together with R³ forms an oxo group;
R⁴ is H or C₁-C₆ alkyl;
R⁵ is iso-butyl, 3,3-dimethylbutyl, thiazolylmethylenyl, hydroxyethylenyl, dichloroethyl, piperidinylmethylenyl, tetrahydropyranylmethylenyl, cyclopropylmethylenyl, cyclohexylmethylenyl, or cyclopentylmethylenyl;
R⁶ is phenyl, phenyl-C₁-C₄-alkylenyl, thienyl, benzo[b]thienyl, benzo[b]furanyl, thieno[2,3-b]pyridinyl, thieno[3,2-b]thienyl, furo[3,2-b]pyridinyl, benzodiazinyl, imidazo[1,2-b]pyridazinyl, indolyl, thienyl-C₁-C₄-alkylenyl, cyclopenta[b]thienyl, C₃-C₇ cycloalkyl, C₃-C₇ cycloalkyl-C₁-C₄ alkylenyl, C₃-C₇ cycloalkyloxy-C₁-C₄ alkylenyl, C₃-C₇ cycloalkylamino, C₁-C₆-alkylamino, C₁-C₆-dialkylamino, N-ethenyl-tetrahydroindolyl, tetrahydroisoquinolinyl, phenylmethyltetrahydroisoquinolinyl, phenylcarbonyltetrahydroisoquinolinyl, or 1,1-dimethylethyldihydroisoquinolincarboxylate-yl, bicyclo[2.2.1]hept-2-yl-C₁-C₄ alkylenyl, wherein R⁶ is optionally substituted with one or more substituents selected from the group consisting of halo, C₁-C₄-alkyl, phenyl, halophenyl, and amino;
R⁷ is H or C₁-C₆ alkyl; and
R⁸ is H, C₁-C₆ alkyl, COOH, acetylamino-C₁-C₄ alkylenyl, or hydroxymethyl.

2. The compound of claim 1, wherein R¹ is phenyl or phenyl substituted with up to three substituents selected from the group consisting of halo, nitro, trifluoromethyl, and cyano; R² is H, methyl, ethyl, n-propyl, isopropyl; n-butyl, 2,2,2-trifluoroethyl, or tetrahydro-2H-pyran-4-yl; R³, R^{3′}, R⁴, R⁷, and R⁸ are H; R⁵ is isobutyl; and R⁶ is benzothien-2-yl.

3. A pharmaceutical composition comprising a compound of claim 1 and a pharmaceutically acceptable carrier, diluent, or excipient.

4. A method comprising the step of administering to a patient in need thereof an effective amount of the compound of claim 1 to treat a disease.

5. The method of claim 4, wherein the disease is selected from the group consisting of: chronic pain, neuropathic pain, postoperative pain, osteoarthritis, neuralgia, neuropathies, algesia, nerve injury, ischaemia, neurodegeneration, cartilage degeneration, and inflammatory disorders.

6. The method of claim 5 wherein the disease is osteoarthritis.

7. A compound selected from the group consisting of:
N-{(1S)-1-[({3-[[(2-chloro-4-cyanophenyl)sulfonyl](methyl)amino]propyl}amino)carbonyl]-3-methylbutyl}-1-benzothiophene-2-carboxamide;
N-[(1S)-1-({[3-({[4-fluoro-2-(trifluoromethyl)phenyl]sulfonyl}amino)propyl]amino}carbonyl)-3-methylbutyl]-1-benzothiophene-2-carboxamide;
N-{(1S)-1-[({3-[[(2,4-dichloro-5-fluorophenyl)sulfonyl](methyl)amino]propyl}amino)carbonyl]-3-methylbutyl}-1-benzothiophene-2-carboxamide;
N-{(1S)-1-[({3-[[(2,4-dibromophenyl)sulfonyl](methyl)amino]propyl}amino)carbonyl]-3-methylbutyl}-1-benzothiophene-2-carboxamide;
N-{(1S)-1-[({3-[[(4-bromo-2-chlorophenyl)sulfonyl](methyl)amino]propyl}amino)carbonyl]-3-methylbutyl}-1-benzothiophene-2-carboxamide;
N-{(1S)-1-[({3-[[(2-chloro-4-fluorophenyl)sulfonyl](methyl)amino]propyl}amino)carbonyl]-3-methylbutyl}-1-benzothiophene-2-carboxamide;
N-{(1S)-1-[({3-[[(2,5-dichlorophenyl)sulfonyl](methyl)amino]propyl}amino)carbonyl]-3-methylbutyl}-1-benzothiophene-2-carboxamide;
N-{(1S)-1-[({3-[[(2-bromo-4-fluorophenyl)sulfonyl](methyl)amino]propyl}amino)carbonyl]-3-methylbutyl}-1-benzothiophene-2-carboxamide;
N-{(1S)-1-[({3-[[(2-chloro-4,5-difluorophenyl)sulfonyl](methyl)amino]propyl}amino)carbonyl]-3-methylbutyl}-1-benzothiophene-2-carboxamide;
N-{(1S)-1-[({3-[[(2,4-difluorophenyl)sulfonyl](methyl)amino]propyl}amino)carbonyl]-3-methylbutyl}-1-benzothiophene-2-carboxamide;
N-{(1S)-1-[({3-[{[2-chloro-4-(trifluoromethyl)phenyl]sulfonyl}(methyl)amino]propyl}amino)carbonyl]-3-methylbutyl}-1-benzothiophene-2-carboxamide;
N-{(1S)-1-[({3-[[(3-bromo-2-thienyl)sulfonyl](methyl)amino]propyl}amino)carbonyl]-3-methylbutyl}-1-benzothiophene-2-carboxamide;
N-{(1S)-1-[({3-[[(3-cyano-2-thienyl)sulfonyl](methyl)amino]propyl}amino)carbonyl]-3-methylbutyl}-1-benzothiophene-2-carboxamide;
N-{(1S)-1-[({3-[[(2,6-dichlorophenyl)sulfonyl](methyl)amino]propyl}amino)carbonyl]-3-methylbutyl}-1-benzothiophene-2-carboxamide;
N-{(1S)-1-[({3-[(2,1,3-benzoxadiazol-4-ylsulfonyl)(methyl)amino]propyl}amino)carbonyl]-3-methylbutyl}-1-benzothiophene-2-carboxamide;
N-{(1S)-1-[({3-[[(2-chloro-6-methylphenyl)sulfonyl](methyl)amino]propyl}amino)carbonyl]-3-methylbutyl}-1-benzothiophene-2-carboxamide;
N-((1S)-3-methyl-1-{[(3-{methyl[(2,4,6-trichlorophenyl)sulfonyl]amino}propyl)amino]carbonyl}butyl)-1-benzothiophene-2-carboxamide;
N-[(1S)-3-methyl-1-({[3-(methyl{[2-(methylsulfonyl)phenyl]sulfonyl}amino)propyl]amino}carbonyl)butyl]-1-benzothiophene-2-carboxamide;
N-{(1S)-1-[({3-[[(2-chloro-4-{[(methylamino)carbonyl]amino}phenyl)sulfonyl](methyl)amino]propyl}amino)carbonyl]-3-methylbutyl}-1-benzothiophene-2-carboxamide;
N-[(1S)-2-({3-[[(2-bromo-4-fluorophenyl)sulfonyl](methyl)amino]propyl}amino)-1-(cyclohexylmethyl)-2-oxoethyl]-1-benzothiophene-2-carboxamide;
N-[(1S)-2-({3-[[(2-chloro-4-fluorophenyl)sulfonyl](methyl)amino]propyl}amino)-1-(cyclohexylmethyl)-2-oxoethyl]-1-benzothiophene-2-carboxamide;
N-[(1S)-2-({3-[[(4-bromo-2-chlorophenyl)sulfonyl](methyl)amino]propyl}amino)-1-(cyclopentylmethyl)-2-oxoethyl]-1-benzothiophene-2-carboxamide;
N-[(1S)-1-(cyclopentylmethyl)-2-({3-[{[4-fluoro-2-(trifluoromethyl)phenyl]sulfonyl}(methyl)amino]propyl}amino)-2-oxoethyl]-1-benzothiophene-2-carboxamide;
N-[(1S)-2-({3-[[(2-bromo-4-fluorophenyl)sulfonyl](methyl)amino]propyl}amino)-1-(cyclopentylmethyl)-2-oxoethyl]-1-benzothiophene-2-carboxamide;
N-[(1S)-2-({3-[[(2-chloro-4-fluorophenyl)sulfonyl](methyl)amino]propyl}amino)-1-(cyclopentylmethyl)-2-oxoethyl]-1-benzothiophene-2-carboxamide;
N-[(1S)-2-({3-[[(6-chloroimidazo[2,1-b][1,3]thiazol-5-yl)sulfonyl](methyl)amino]propyl}amino)-1-(cyclopentylmethyl)-2-oxoethyl]-1-benzothiophene-2-carboxamide;
N-[(1S)-2-({3-[[(4-chloro-2-fluorophenyl)sulfonyl](methyl)amino]propyl}amino)-1-(cyclopentylmethyl)-2-oxoethyl]-1-benzothiophene-2-carboxamide;
N-[(1S)-2-({3-[[(4-bromo-2-fluorophenyl)sulfonyl](methyl)amino]propyl}amino)-1-(cyclopentylmethyl)-2-oxoethyl]-1-benzothiophene-2-carboxamide;
N-[(1S)-2-({3-[[(4-cyanophenyl)sulfonyl](methyl)amino]propyl}amino)-1-(cyclopentylmethyl)-2-oxoethyl]-1-benzothiophene-2-carboxamide;
N-((1S)-1-(cyclopentylmethyl)-2-{[3-(methyl{[2-(trifluoromethyl)phenyl]sulfonyl}amino)propyl]amino}-2-oxoethyl)-1-benzothiophene-2-carboxamide;
N-[(1S)-2-({3-[[(4-chloro-2,5-difluorophenyl)sulfonyl](methyl)amino]propyl}amino)-1-(cyclopentylmethyl)-2-oxoethyl]-1-benzothiophene-2-carboxamide;
N-[(1S)-1-(cyclopentylmethyl)-2-({3-[[(2,3-dichlorophenyl)sulfonyl](methyl)amino]propyl}amino)-2-oxoethyl]-1-benzothiophene-2-carboxamide;
N-[(1S)-2-({3-[[(6-chloroimidazo[2,1-b][1,3]thiazol-5-yl)sulfonyl](methyl)amino]propyl}amino)-1-(cyclopentylmethyl)-2-oxoethyl]-1-benzothiophene-2-carboxamide;
5-bromo-N-{(1S)-1-[({3-[[(2-chloro-4-fluorophenyl)sulfonyl](methyl)amino]propyl}amino)carbonyl]-3-methylbutyl}-1-benzothiophene-2-carboxamide;
N-((1S)-1-{[(3-{[(2-chloro-4-fluorophenyl)sulfonyl]amino}propyl)amino]carbonyl}-3-methylbutyl)-5,6-dihydro-4H-cyclopenta[b]thiophene-2-carboxamide;
1,1-dimethylethyl 6-{[((1S)-1-{[(3-{[(2-chloro-4-fluorophenyl)sulfonyl]amino}propyl)amino]carbonyl}-3-methylbutyl)amino]carbonyl}-3,4-dihydro-2(1H)-isoquinolinecarboxylate;
1,1-dimethylethyl 5-{[((1S)-1-{[(3-{[(2-chloro-4-fluorophenyl)sulfonyl]amino}propyl)amino]carbonyl}-3-methylbutyl)amino]carbonyl}-3,4-dihydro-2(1H)-isoquinolinecarboxylate;
N-{(1S)-1-[({3-[[(2-chloro-4-fluorophenyl)sulfonyl](methyl)amino]propyl}amino)carbonyl]-3-methylbutyl}-4-fluoro-1-benzothiophene-2-carboxamide;
N¹-(3-{[(2-chloro-4-fluorophenyl)sulfonyl]amino}propyl)-N²-(5-cyclohexylpentanoyl)-L-leucinamide;
N¹-(3-{[(2-chloro-4-fluorophenyl)sulfonyl]amino}propyl)-N²-(4-cyclohexylbutanoyl)-L-leucinamide;
1,1-dimethylethyl 7-{[((1S)-1-{[(3-{[(2-chloro-4-fluorophenyl)sulfonyl]amino}propyl)amino]carbonyl}-3-methylbutyl)amino]carbonyl}-3,4-dihydro-2(1H)-isoquinolinecarboxylate;
N¹-(3-{[(2-chloro-4-fluorophenyl)sulfonyl]amino}propyl)-N²-({[(1S,2S,5R)-5-methyl-2-(1-methylethyl)cyclohexyl]oxy}acetyl)-L-leucinamide;
N-((1S)-1-{[(3-{[(2-chloro-4-fluorophenyl)sulfonyl]amino}propyl)amino]carbonyl}-3-methylbutyl)-1-ethenyl-4,5,6,7-tetrahydro-1H-indole-2-carboxamide;
3-chloro-N-{(1S)-1-[({3-[[(2-chloro-4-fluorophenyl)sulfonyl](methyl)amino]propyl}amino)carbonyl]-3-methylbutyl}-6-fluoro-1-benzothiophene-2-carboxamide;
N¹-(3-{[(2-chloro-4-fluorophenyl)sulfonyl]amino}propyl)-N²-(cyclopentylacetyl)-L-leucinamide;
N²-[(1S,4R)-bicyclo[2.2.1]hept-2-ylacetyl]-N¹-(3-{[(2-chloro-4-fluorophenyl)sulfonyl]amino}propyl)-L-leucinamide;
3-chloro-N-((1S)-1-{[(3-{[(2-chloro-4-fluorophenyl)sulfonyl]amino}propyl)amino]carbonyl}-3-methylbutyl)-6-fluoro-1-benzothiophene-2-carboxamide;
3-chloro-N-{(1S)-1-[({3-[[(2-chloro-4-fluorophenyl)sulfonyl](methyl)amino]propyl}amino)carbonyl]-3-methylbutyl}-6-methyl-1-benzothiophene-2-carboxamide;
3,4-dichloro-N-{(1S)-1-[({3-[[(2-chloro-4-fluorophenyl)sulfonyl](methyl)amino]propyl}amino)carbonyl]-3-methylbutyl}-1-benzothiophene-2-carboxamide;
N¹-(3-{[(2-chloro-4-fluorophenyl)sulfonyl]amino}propyl)-N²-(3-phonylpropanoyl)-L-leucinamide;
N²-(1-benzothien-2-ylacetyl)-N¹-(3-{[(2-chloro-4-fluorophenyl)sulfonyl]amino}propyl)-L-leucinamide;
N¹-(3-{[(2-chloro-4-fluorophenyl)sulfonyl]amino}propyl)-N²-[3-(2-thienyl)propanoyl]-L-leucinamide;
1,1-dimethylethyl 8-{[((1S)-1-{[(3-{[(2-chloro-4-fluorophenyl)sulfonyl]amino}propyl)amino]carbonyl}-3-methylbutyl)amino]carbonyl}-3,4-dihydro-2(1H)-isoquinolinecarboxylate;
N¹-(3-{[(2-chloro-4-fluorophenyl)sulfonyl]amino}propyl)-N²-(cyclopentylcarbonyl)-L-leucinamide;
N¹-(3-{[(2-chloro-4-fluorophenyl)sulfonyl]amino}propyl)-N²-[(5-fluoro-1H-indol-3-yl)acetyl]-L-leucinamide;
N¹-(3-{[(2-chloro-4-fluorophenyl)sulfonyl]amino}propyl)-N²-(3-cyclopropylpropanoyl)-L-leucinamide;
N¹-(3-{[(2-chloro-4-fluorophenyl)sulfonyl]amino}propyl)-N²-[3-(1H-indol-2-yl)propanoyl]-L-leucinamide;
N-((1S)-1-{[(3-{[(2-chloro-4-fluorophenyl)sulfonyl]amino}propyl)amino]carbonyl}-3-methylbutyl)-6-quinoxalinecarboxamide;
3-amino-N-((1S)-1-{[(3-{[(2-chloro-4-fluorophenyl)sulfonyl]amino}propyl)amino]carbonyl}-3-methylbutyl)thieno[2,3-b]pyridine-2-carboxamide;
3-cyclopentyl-N-((1S)-2-{[3-({[4-fluoro-2-(trifluoromethyl)phenyl]sulfonyl}amino)propyl]amino}-2-oxo-1-phenylethyl)propanamide;
N²-(3-cyclohexylpropanoyl)-N¹-[3-({[4-fluoro-2-(trifluoromethyl)phenyl]sulfonyl}amino)propyl]-L-leucinamide;
N²-(3-cyclopentylpropanoyl)-N¹-[3-({[4-fluoro-2-(trifluoromethyl)phenyl]sulfonyl}amino)propyl]-L-leucinamide;
N-((1S)-1-{[(3-{[(2-chloro-4-fluorophenyl)sulfonyl]amino}propyl)amino]carbonyl}-3-methylbutyl)-1,2,3,4-tetrahydro-5-isoquinolinecarboxamide;
N-((1S)-1-{[(3-{[(2-chloro-4-fluorophenyl)sulfonyl]amino}propyl)amino]carbonyl}-3-methylbutyl)-2-(phenylmethyl)-1,2,3,4-tetrahydro-5-isoquinolinecarboxamide;
N-((1S)-1-{[(3-{[(2-chloro-4-fluorophenyl)sulfonyl]amino}propyl)amino]carbonyl}-3-methylbutyl)-2-(phenylcarbonyl)-1,2,3,4-tetrahydro-5-isoquinolinecarboxamide;
N-{(1S)-1-[({3-[[(cyanophenyl)sulfonyl](methyl)amino]propyl}amino)carbonyl]-3-methylbutyl}-1-benzothiophene-2-carboxamide;
N-{(1S)-1-[({3-[[(2-cyanophenyl)sulfonyl](methyl)amino]propyl}amino)carbonyl]-3-methylbutyl}thieno[3,2-b]thiophene-2-carboxamide;
N-{(1S)-1-[({3-[[(2-cyanophenyl)sulfonyl](methyl)amino]propyl}amino)carbonyl]-3-methylbutyl}-1-methyl-1H-indole-2-carboxamide;
N-{(1S)-1-[({3-[[(2-cyanophenyl)sulfonyl](methyl)amino]propyl}amino)carbonyl]-3-methylbutyl}-1-benzofuran-2-carboxamide;
N²-{[2-(3-chlorophenyl)-1,3-thiazol-5-yl]carbonyl}-N¹-{3-[[(2-cyanophenyl)sulfonyl](methyl)amino]propyl}-L-leucinamide;
6-chloro-N-{(1S)-1-[({3-[[(2-cyanophenyl)sulfonyl](methyl)amino]propyl}amino)carbonyl]-3-methylbutyl}imidazo[1,2-b]pyridazine-2-carboxamide;
N-{(1S)-1-[({3-[[(2-cyanophenyl)sulfonyl](methyl)amino]propyl}amino)carbonyl]-3-methylbutyl}-5-methyl-1-benzothiophene-2-carboxamide;
N-{(1S)-1-[({3-[[(2-cyanophenyl)sulfonyl](methyl)amino]propyl}amino)carbonyl]-3-methylbutyl}-3-methylfuro[3,2-b]pyridine-2-carboxamide;
N-[(1S)-2-({3-[[(2-cyanophenyl)sulfonyl](methyl)amino]propyl}amino)-1-(cyclopentylmethyl)-2-oxoethyl]-1-benzothiophene-2-carboxamide;
N-[(1S)-2-({3-[[(2-cyanophenyl)sulfonyl](methyl)amino]propyl}amino)-1-(cyclohexylmethyl)-2-oxoethyl]-1-benzothiophene-2-carboxamide;
N-{(1S)-1-[({3-[[(2-cyanophenyl)sulfonyl](methyl)amino]propyl}amino)carbonyl]-3,3-dimethylbutyl}-1-benzothiophene-2-carboxamide;
N-{(1S)-1-[({3-[[(2-cyanophenyl)sulfonyl](ethyl)amino]propyl}amino)carbonyl]-3-methylbutyl}-1-benzothiophene-2-carboxamide;
N-{(1S)-1-[({3-[[(2-cyanophenyl)sulfonyl](1-methylethyl)amino]propyl}amino)carbonyl]-3-methylbutyl}-1-benzothiophene-2-carboxamide;
N-{(1S)-1-[({3-[[(2-cyanophenyl)sulfonyl](propyl)amino]propyl}amino)carbonyl]-3-methylbutyl}-1-benzothiophene-2-carboxamide;
N-{(1S)-1-[({3-[[(2-cyanophenyl)sulfonyl](cyclopropyl)amino]propyl}amino)carbonyl]-3-methylbutyl}-1-benzothiophene-2-carboxamide;
N-((1S)-1-{[(3{cyclopropyl[(2,4dichlorophenyl)sulfonyl]amino}propyl)amino]carbonyl}-3-methylbutyl)-1-benzothiophene-2-carboxamide;
N-{(1S)-1-[({3-[[(2-cyanophenyl)sulfonyl](tetrahydro-2H-pyran-4-yl)amino]propyl}amino)carbonyl]-3-methylbutyl}-1-benzothiophene-2-carboxamide;
N-{(1S)-1-[({3-[[(2-cyanophenyl)sulfonyl](2,2,2-trifluoroethyl)amino]propyl}amino)carbonyl]-3-methylbutyl}-1-benzothiophene-2-carboxamide;
N-{(1S)-1-[({3-[[(2,4-dichlorophenyl)sulfonyl](methyl)amino]propyl}amino)carbonyl]-3-methylbutyl}-1-benzothiophene-2-carboxamide;
N-{(1S)-3-methyl-1-[({3-[methyl(2-thienylsulfonyl)amino]propyl}amino)carbonyl]butyl}-1-benzothiophene-2-carboxamide;
N-{(1S)-3-methyl-1-[({3-[methyl(2-thienylsulfonyl)amino]propyl}amino)carbonyl]butyl}-1-benzothiophene-2-carboxamide;
N-{(1S)-1-[({3-[[(3-bromo-5-chloro-2-thienyl)sulfonyl](methyl)amino]propyl}amino)carbonyl]-3-methylbutyl}-1-benzothiophene-2-carboxamide;
methyl 4-{[(3-{[N-(1-benzothien-2-ylcarbonyl)-L-leucyl]amino}propyl)(methyl)amino]sulfonyl}-2,5-dimethyl-3-furancarboxylate;
N-{(1S)-1-[({3-[[(4-chlorophenyl)sulfonyl](methyl)amino]propyl}amino)carbonyl]-3-methylbutyl}-1-benzothiophene-2-carboxamide;
N-{(1S)-1-[({3-[[(2-chlorophenyl)sulfonyl](methyl)amino]propyl}amino)carbonyl]-3-methylbutyl}-1-benzothiophene-2-carboxamide;
N-{(1S)-1-[({3-[[(2-bromophenyl)sulfonyl](methyl)amino]propyl}amino)carbonyl]-3-methylbutyl}-1-benzothiophene-2-carboxamide;
N-{(1S)-1-[({3-[({4-bromo-2-[(trifluoromethyl)oxy]phenyl}sulfonyl)(methyl)amino]propyl}amino)carbonyl]-3-methylbutyl}-1-benzothiophene-2-carboxamide;
N-{(1S)-1-[({3-[({4-bromo-2-[(trifluoromethyl)oxy]phenyl}sulfonyl)(methyl)amino]propyl}amino)carbonyl]-3-methylbutyl}-1-benzothiophene-2-carboxamide;
1,1-Dimethylethyl (3-{[N-(1-benzothien-2-ylcarbonyl)-L-leucyl]amino}propyl)carbamate;
N-((1S)-1-{[(3-{[(2,4-Dichlorophenyl)sulfonyl]amino}propyl)amino]carbonyl}-3-methylbutyl)-1-benzothiophene-2-carboxamide;
N-((1S)-1-{[(3-{[(2-Chloro-4-fluorophenyl)sulfonyl]amino}propyl)amino]carbonyl}-3-methylbutyl)-1-benzothiophene-2-carboxamide;
N-((1S)-1-{[(3-{[(2-Chloro-4-fluorophenyl)carbonyl]amino}propyl)amino]carbonyl}-3-methylbutyl)-1-benzothiophene-2-carboxamide;
N-((1S)-1-{[{3-[[(2-Cyanophenyl)sulfonyl](methyl)amino]propyl}(methyl)amino]carbonyl}-3-methylbutyl)-1-methyl-1H-indole-2-carboxamide;
N-[(1S)-2-[(4-{[(2,4-Dichlorophenyl)sulfonyl]amino}butyl)amino]-2-oxo-1-(1,3-thiazol-4-ylmethyl)ethyl]-1-benzothiophene-2-carboxamide;
N-((1S)-1-{[(3-{[(2,4-Dichlorophenyl)sulfonyl]amino}propyl)amino]carbonyl}-3-hydroxypropyl)-1-benzothiophene-2-carboxamide;
N-((1S)-3,3-Dichloro-1-{[(3-{[(2,4-dichlorophenyl)sulfonyl]amino}propyl)amino]carbonyl}propyl)-1-benzothiophene-2-carboxamide;
N-{(1S)-1-[({(2R)-3-[[(2-cyanophenyl)sulfonyl](methyl)amino]-2-hydroxypropyl}amino)carbonyl]-3-methylbutyl}-1-benzothiophene-2-carboxamide;
N-[(1S)-2-({(2R)-3-[[(2-cyanophenyl)sulfonyl](methyl)amino]-2-hydroxypropyl}amino)-1-(cyclohexylmethyl)-2-oxoethyl]-1-benzothiophene-2-carboxamide;
N-[(1S)-1-(cyclohexylmethyl)-2-({(2R)-3-[[(2,4-dichlorophenyl)sulfonyl](methyl)amino]-2-hydroxypropyl}amino)-2-oxoethyl]-1-benzothiophene-2-carboxamide;
N-[(1S)-2-({(2R)-3-[[(2-cyanophenyl)sulfonyl](methyl)amino]-2-hydroxypropyl}amino)-1-(cyclopentylmethyl)-2-oxoethyl]-1-benzothiophene-2-carboxamide;
N-[(1S)-1-(cyclopentylmethyl)-2-({(2R)-3-[[(2,4-dichlorophenyl)sulfonyl](methyl)amino]-2-hydroxypropyl}amino)-2-oxoethyl]-1-benzothiophene-2-carboxamide;
N-{(1S)-1-[({(2R)-3-[[(2-chloro-4-fluorophenyl)sulfonyl](methyl)amino]-2-hydroxypropyl}amino)carbonyl]-3-methylbutyl}-1-benzothiophene-2-carboxamide;
N-[(1S)-2-({(2R)-3-[[(2-chloro-4-fluorophenyl)sulfonyl](methyl)amino]-2-hydroxypropyl}amino)-1-(cyclohexylmethyl)-2-oxoethyl]-1-benzothiophene-2-carboxamide;
N-{(1S)-1-[({(2R)-3-[[(2,4-dichlorophenyl)sulfonyl](methyl)amino]-2-hydroxypropyl}amino)carbonyl]-3-methylbutyl}-1-benzothiophene-2-carboxamide;
N-{(1S)-1-[({(2S)-3-[[(2,4-dichlorophenyl)sulfonyl](methyl)amino]-2-hydroxypropyl}amino)carbonyl]-3-methylbutyl}-1-benzothiophene-2-carboxamide;
N-((1S)-1-{[((2S)-2-hydroxy-3-{methyl[(2-nitrophenyl)sulfonyl]amino}propyl)amino]carbonyl}-3-methylbutyl)-1-benzothiophene-2-carboxamide;
N-((1S)-1-{[((2R)-2-hydroxy-3-{methyl[(2-nitrophenyl)sulfonyl]amino}propyl)amino]carbonyl}-3-methylbutyl)-1-benzothiophene-2-carboxamide;
N-{(1S)-1-[({(2S)-3-[[(2-cyanophenyl)sulfonyl](methyl)amino]-2-hydroxypropyl}amino)carbonyl]-3-methylbutyl}-1-benzothiophene-2-carboxamide;
N-{(1S)-1-[({3-[[(2,4-dichlorophenyl)sulfonyl](methyl)amino]-2-oxopropyl}amino)carbonyl]-3-methylbutyl}-1-benzothiophene-2-carboxamide;
N-[(1S)-1-({[3-[[(2,4-dichlorophenyl)sulfonyl](methyl)amino]-2-(methylamino)propyl]amino}carbonyl)-3-methylbutyl]-1-benzothiophene-2-carboxamide;
N-{(1S)-1-(cyclohexylmethyl)-2-[((2R)-2-hydroxy-3-{methyl[(2-nitrophenyl)sulfonyl]amino}propyl)amino]-2-oxoethyl}-1-benzothiophene-2-carboxamide;
N-[(1S)-2-({(2R)-3-[[(2-cyanophenyl)sulfonyl](methyl)amino]-2-hydroxypropyl}amino)-1-(cyclohexylmethyl)-2-oxoethyl]-1-methyl-1H-indole-2-carboxamide;
N-{(1S)-1-(cyclopentylmethyl)-2-[((2R)-2-hydroxy-3-{methyl[(2-nitrophenyl)sulfonyl]amino}propyl)amino]-2-oxoethyl}-1-benzothiophene-2-carboxamide;
N-[(1S)-1-({[3-[[(2,4-dichlorophenyl)sulfonyl](methyl)amino]-2-(methyloxy)propyl]amino}carbonyl)-3-methylbutyl]-1-benzothiophene-2-carboxamide;
N-[(1S)-1-({[3-[[(2,4-dichlorophenyl)sulfonyl](methyl)amino]-2-(2-propen-1-yloxy)propyl]amino}carbonyl)-3-methylbutyl]-1-benzothiophene-2-carboxamide;
N-((1S)-1-{[((2R)-3-{[(2-chloro-4-fluorophenyl)sulfonyl]amino}-2-hydroxypropyl)amino]carbonyl}-3-methylbutyl)-1-benzothiophene-2-carboxamide;
N-((1S)-1-{[((2R)-3-{[(2,4-dichlorophenyl)sulfonyl]amino}-2-hydroxypropyl)amino]carbonyl}-3-methylbutyl)-1-benzothiophene-2-carboxamide;
N-[(1S)-1-({[(2R)-3-({[4-fluoro-2-(trifluoromethyl)phenyl]sulfonyl}amino)-2-hydroxypropyl]amino}carbonyl)-3-methylbutyl]-1-benzothiophene-2-carboxamide;
N-[(1S)-2-[((2R)-3-{[(2-chloro-4-fluorophenyl)sulfonyl]amino}-2-hydroxypropyl)amino]-1-(cyclohexylmethyl)-2-oxoethyl]-1-benzothiophene-2-carboxamide;
N²-[(cyclohexylamino)carbonyl]-N¹-((2R)-3-{[(2,4-dichlorophenyl)sulfonyl]amino}-2-hydroxypropyl)-L-leucinamide;
N-((1S)-1-{[(3-{[(2,4-dichlorophenyl)sulfonyl]amino}-2-oxopropyl)amino]carbonyl}-3-methylbutyl)-1-benzothiophene-2-carboxamide;
N-((1S)-1-{[(3-{[(2-chloro-4-fluorophenyl)sulfonyl]amino}-2-oxopropyl)amino]carbonyl}-3-methylbutyl)-1-benzothiophene-2-carboxamide;
N-((1S)-1-{[(3-{[(4-Chlorophenyl)sulfonyl]amino}-1-ethylpropyl)amino]carbonyl}-3-methylbutyl)-1-benzothiophene-2-carboxamide;
N-((1S)-1-{[(1-Ethyl-3-{[(2-nitrophenyl)sulfonyl]amino}propyl)amino]carbonyl}-3-methylbutyl)-1-benzothiophene-2-carboxamide;
N-{(1S)-1-[({3-[[(4-Chlorophenyl)sulfonyl](methyl)amino]-1-ethylpropyl}amino)carbonyl]-3-methylbutyl}-1-benzothiophene-2-carboxamide;
N-((1S)-1-{[(1-Ethyl-3-{[(2-nitrophenyl)sulfonyl](methyl)amino}propyl)amino]carbonyl}-3-methylbutyl)-1-benzothiophene-2-carboxamide;
N-{(1S)-1-[({3-[[(2-Cyanophenyl)sulfonyl](methyl)amino]-1-ethylpropyl}amino)carbonyl]-3-methylbutyl}-1-benzothiophene-2-carboxamide;
N-((1S)-1-{[(3-{[(2-Chloro-4-fluorophenyl)sulfonyl]amino}pentyl)amino]carbonyl}-3-methylbutyl)-1-benzothiophene-2-carboxamide;
N-((1S)-1-{[(3-{[(2,4-Dichlorophenyl)sulfonyl]amino}pentyl)amino]carbonyl}-3-methylbutyl)-1-benzothiophene-2-carboxamide;
N-((1S)-1-{[((3R)-3-{[(2-Chloro-4-fluorophenyl)sulfonyl]amino}-4-hydroxybutyl)amino]carbonyl}-3-methylbutyl)-1-benzothiophene-2-carboxamide;
N-((1S)-1-{[((3S)-3-{[(2-Chloro-4-fluorophenyl)sulfonyl]amino}-4-hydroxybutyl)amino]carbonyl}-3-methylbutyl)-1-benzothiophene-2-carboxamide;
Methyl (2R)-4-{[N-(1-benzothien-2-ylcarbonyl)-L-leucyl]amino}-2-{[(2-chloro-4-fluorophenyl)sulfonyl]amino}butanoate;
N-((1S)-1-{[((3S)-3-{[(2-chloro-4-fluorophenyl)sulfonyl]amino}butyl)amino]carbonyl}-3-methylbutyl)-1-benzothiophene-2-carboxamide;
N-{(1S)-1-[({3-[[(2-Cyanophenyl)sulfonyl](methyl)amino]propyl}amino)carbonyl]-3-methylbutyl}-3-methyl-1-benzothiophene-2-carboxamide;
5-Chloro-N-{(1S)-1-[({3-[[(2-cyanophenyl)sulfonyl](methyl)amino]propyl}amino)carbonyl]-3-methylbutyl}-3-methyl-1-benzothiophene-2-carboxamide;
N¹-{3-[[(2-Cyanophenyl)sulfonyl](methyl)amino]propyl}-N²-(2-thienylcarbonyl)-L-leucinamide;
N-[(1S)-2-({3-[[(2-Cyanophenyl)sulfonyl](methyl)amino]propyl}amino)-1-(cyclopropylmethyl)-2-oxoethyl]-1-benzothiophene-2-carboxamide;
1,1-Dimethylethyl 4-[(2S)-2-[(1-benzothien-2-ylcarbonyl)amino]-3-({3-[[(2-cyanophenyl)sulfonyl](methyl)amino]propyl}amino)-3-oxopropyl]-1-piperidinecarboxylate;
N-[2-({3-[[(2-Cyanophenyl)sulfonyl](methyl)amino]propyl}amino)-2-oxo-1-(tetrahydro-2H-pyran-4-ylmethyl)ethyl]-1-benzothiophene-2-carboxamide;
N-((1S)-1-{[(3-{[(2-cyanophenyl)sulfonyl][2-(dimethylamino)ethyl]amino}propyl)amino]carbonyl}-3-methylbutyl)-1-benzothiophene-2-carboxamide;
N-((1S)-1-{[(3-{[(2-cyanophenyl)sulfonyl][2-(methyloxy)ethyl]amino}propyl)amino]carbonyl}-3-methylbutyl)-1-benzothiophene-2-carboxamide;
N-{(1S)-1-[({3-[[(2-cyanophenyl)sulfonyl](methyloxy)amino]propyl}amino)carbonyl]-3-methylbutyl}-1-benzothiophene-2-carboxamide;
N-{(1S)-1-[({3-[[(2-cyanophenyl)sulfonyl](2-pyridinylmethyl)amino]propyl}amino)carbonyl]-3-methylbutyl}-1-benzothiophene-2-carboxamide;
N-((1S)-1-{[(3-{butyl[(2-cyanophenyl)sulfonyl]amino}propyl)amino]carbonyl}-3-methylbutyl)-1-benzothiophene-2-carboxamide;
N-((1S)-1-{[(3-{butyl[(2,4-dichlorophenyl)sulfonyl]amino}propyl)amino]carbonyl}-3-methylbutyl)-1-benzothiophene-2-carboxamide;
N-[(1S)-2-({3-[[(2-cyanophenyl)sulfonyl](cyclopropyl)amino]propyl}amino)-1-(cyclopentylmethyl)-2-oxoethyl]-1-benzothiophene-2-carboxamide;
N-((1S)-1-{[(3-{[(2-chloro-4-fluorophenyl)sulfonyl]amino}-2,2-dimethylpropyl)amino]carbonyl}-3-methylbutyl)-1-benzothiophene-2-carboxamide;
N-{(1S)-1-[({(2R)-3-[[(2-chlorophenyl)sulfonyl](methyl)amino]-2-hydroxypropyl}amino)carbonyl]-3-methylbutyl}-1-benzothiophene-2-carboxamide;
N-{(1S)-1-[({(2R)-3-[[(4-chlorophenyl)sulfonyl](methyl)amino]-2-hydroxypropyl}amino)carbonyl]-3-methylbutyl}-1-benzothiophene-2-carboxamide;
N-{(1S)-1-[({(2R)-3-[[(2-cyanophenyl)sulfonyl](methyl)amino]-2-fluoropropyl}amino)carbonyl]-3-methylbutyl}-1-benzothiophene-2-carboxamide;
N-{(1S)-1-[({(2R)-3-[[(2,4-dichlorophenyl)sulfonyl](methyl)amino]-2-fluoropropyl}amino)carbonyl]-3-methylbutyl}-1-benzothiophene-2-carboxamide;
N-{(1S)-1-[({(2R)-3-[[(2-chloro-4-fluorophenyl)sulfonyl](methyl)amino]-2-fluoropropyl}amino)carbonyl]-3-methylbutyl}-1-benzothiophene-2-carboxamide;
N-((1S)-1-{[((3R)-4-(Acetylamino)-3-{[(2-chloro-4-fluorophenyl)sulfonyl]amino}butyl)amino]carbonyl}-3-methylbutyl)-1-benzothiophene-2-carboxamide;
N-((1S)-1-{[((2R,3S)-3-{[(2-chloro-4-fluorophenyl)sulfonyl]amino}-2,4-dihydroxybutyl)amino]carbonyl}-3-methylbutyl)-1-benzothiophene-2-carboxamide; and
5-{[N-(1-benzothien-2-ylcarbonyl)-L-leucyl]amino}-3-{[(2-chloro-4-fluorophenyl)sulfonyl]amino}-1,2,3,5-tetradeoxy-D-erythro-pentitol.
